(19) United States
(12) Patent Application Publication
(10) Pub. No.: US 2023/0026271 A1
(43) Pub. Date: Jan. 26, 2023

(54) Piperazine Compounds for Inhibiting CPS1

(71) Applicant: Eisai R&D Management Co., Ltd., Bunkyo-ku, Tokyo (JP)

(72) Inventors: Alan ROLFE, Medford, MA (US); David BOLDUC, Milton, MA (US); Nicholas LARSEN, Needham, MA (US); Andrew S. COOK, Stow, MA (US); Kiyoyuki OMOTO, Bedford, MA (US)

(73) Assignee: Eisai R&D Management Co., Ltd., Bunkyo-ku, Tokyo (JP)

(21) Appl. No.: 17/778,635

(22) PCT Filed: Nov. 20, 2020
(86) PCT No.: PCT/US2020/061567

§ 371 Date: May 20, 2022

Related U.S. Application Data
(60) Provisional application No. 62/939,228, filed on Nov. 22, 2019.

(51) Int. Cl. C07D 241/04; C07D 403/06; C07D 405/06; C07D 413/06

(52) U.S. Cl. CPC C07D 241/04; C07D 403/06; C07D 405/06; C07D 413/06

(57) ABSTRACT

We provide compounds that may be useful as CPS1 inhibitors. These compounds may be useful, for example, in the treatment of cancer.

### CROSS-REFERENCE TO RELATED APPLICATIONS

This application claims the benefit of U.S. Provisional Patent Application No. 62/939,228, filed on Nov. 22, 2019. That application is incorporated by reference as if fully rewritten herein.

### BACKGROUND

The primary function of the urea cycle is to remove toxic ammonia from the blood. That ammonia is produced as a normal byproduct of amino acid catabolism. (Adeva, M. M., Souto, G., Blanco, N. & Donapetry, C. Ammonium metabolism in humans. Metabolism 61, 1495-1511 (2012)). In cancer, malignant cells have found ways to utilize this pathway to support tumor growth. (Keshet, R., et al., Rewiring urea cycle metabolism in cancer to support anabolism. Nat Rev Cancer 18, 634-645 (2018); Lee, J. S. et al. Urea Cycle Dysregulation Generates Clinically Relevant Genomic and Biochemical Signatures. Cell 174, 1559-1570.e22 (2018); Rabinovich, S. et al. Diversion of aspartate in ASS1-deficient tumours fosters de novo pyrimidine synthesis. Nature 527, 379-383 (2015); Li, L. et al. p53 regulation of ammonia metabolism through urea cycle controls polyamine biosynthesis. Nature 567, 253-256 (2019); Qelikta, M. et al. Role of CPS1 in Cell Growth, Metabolism, and Prognosis in LKB1-Inactivated Lung Adenocarcinoma. JNCI J Natl Cancer Inst 109, djw231 (2017); Kim, J. et al. CPS1 maintains pyrimidine pools and DNA synthesis in KRAS/LKB1-mutant lung cancer cells. Nature 546, 168-172 (2017); Pham-Danis, C. et al. Urea Cycle Sustains Cellular Energetics upon EGFR Inhibition in EGFR-Mutant NSCLC. Mol. Cancer Res. 17, 1351-1364 (2019)).

This is accomplished primarily through two mechanisms. One mechanism is repurposing of urea cycle metabolites to sustain anabolic pathways, mostly in the support of pyrimidine synthesis. A second mechanism is up regulation of urea cycle activity to prevent the accumulation of toxic ammonia in growing tumors.

Catalyzing the first and rate limiting step in ammonia commitment to the urea cycle, upregulation of carbamoyl phosphate synthetase 1 (CPS1) activity plays an important role in supporting tumor growth through these mechanisms. CPS1 overexpression and activity has been demonstrated to promote tumor growth through the support of pyrimidine synthesis in a number of cancer types. (Keshet, et al.; Lee, et al.; Qelikta, M. et al.; Kim, J. et al.) In this setting, carbamoyl phosphate generated by CPS1 circumvents the urea cycle and is instead consumed by the CAD gene in the pyrimidine biosynthetic pathway. This rewired pathway allows cancer cells to by pass a negative feedback loop of pyrimidine inhibition of CPS2 function in the pyrimidine biosynthetic pathway that would otherwise limit pyrimidine for DNA synthesis in rapidly growing malignant cells. In p53 deficient cancers, CPS1 overexpression has been shown to be required for the removal of toxic ammonia from tumors to allow for sustained growth. (Li, L. et al.)

Up regulation of CPS1 expression occurs in several cancer types and has been associated with poor prognosis in lung, colon, cholangiocarcinoma and some subtypes of glioblastoma. (Kim, J. et al.; Palaniappan, A., et al., Computational Identification of Novel Stage-Specific Biomarkers in Colorectal Cancer Progression. PLoS ONE 11, e0156665 (2016); May, D. et al. Investigating Neoplastic Progression of Ulcerative Colitis with Label-Free Comparative Proteomics. J. Proteome Res. 10, 200-209 (2011); Lee, Y.-Y. et al. Overexpression of CPS1 is an independent negative prognosticator in rectal cancers receiving concurrent chemoradiotherapy. Tumor Biol. 35, 11097-11105 (2014); Ma, S.-L., et al. Co-expression of the carbamoyl-phosphate synthase 1 gene and its long non-coding RNA correlates with poor prognosis of patients with intrahepatic cholangiocarcinoma. Molecular Medicine Reports 12, 7915-7926 (2015); Milinkovic, V. et al. Identification of Novel Genetic Alterations in Samples of Malignant Glioma Patients. PLoS ONE 8, e82108 (2013)). These observations highlight the need for the identification of small molecule inhibitors to probe CPS1 cancer biology, with the hope that targeted therapeutics can be developed to treat cancers that depend on CPS1 for growth.

In the human genome, there are two known genes with carbamoyl phosphate synthesis activity, CPS1 and CAD. (de Cima, S. et al. Structure of human carbamoyl phosphate synthetase: deciphering the on/off switch of human ureagenesis. Sci Rep 5, 16950 (2015); Shaw, S. M. & Carrey, E. A. Regulation of the mammalian carbamoyl-phosphate synthetase II by effectors and phosphorylation. Altered affinity for ATP and magnesium ions measured using the ammonia-dependent part reaction. Eur. J. Biochem. 207, 957-965 (1992)).

CAD is a trifunctional enzyme, housing CPS2, Apartyl transcarbamoylase and Dihydroorotase activity. Under normal physiologic conditions, CPS1 activity is involved exclusively with ammonia detoxification through the urea cycle in the mitochondria, while CAD activity in the cytoplasm catalyzes the first three steps in de novo pyrimidine synthesis. When considering the development of CPS1 targeted inhibitors for the treatment of cancer, selectivity for CPS1 over the CPS2 activity of CAD is a top priority to avoid toxicities associated with systemic inhibition of pyrimidine synthesis.

The CPS1 protein is comprised of six globular domains: A small N-terminal domain of unknown function, a functionally inactive glutamine amido transferase domain, two unique ATPase domains, an integrating domain and an N-acetyl glutamate (NAG) binding domain. (de Cima, S. et al.) CPS1 synthesizes carbamoyl phosphate from ammonia, bicarbonate and two molecules of ATP. This is achieved through the coordinated efforts of its two separate and functionally distinct ATPase domains. At the first ATPase domain, bicarbonate is first phosphorylated generating carboxy phosphate. The phosphate group of carboxy phosophate is then displace by ammonia, yielding carbamate, which is subsequently shuttled through an internal tunnel to the second ATPase domain. A phosphate from a second molecule of ATP is then transferred to carbamate to produce the final product, carbamoyl phosphate.

CPS1 activity is stimulated by a small molecule co-factor, N-acetyl glutamate (NAG), which binds the NAG binding domain. Upon NAG binding, large conformational changes occur in the ATPase domains that allows for efficient catalysis. The integrating domain separates the two ATPase domains and likely contributes to the stability of the catalytically competent CPS1 conformation.

### BRIEF SUMMARY

Described herein are novel compounds useful for treatment of cancer, particularly cancer exhibiting altered CPS1 status, and very particularly cancer exhibiting increased expression of CPS1 relative to non-cancerous cells from the subject requiring treatment for cancer.

Embodiments may include, for example, a compound given by Formula I

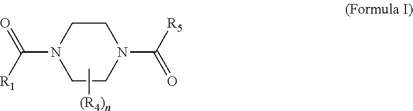

or a pharmaceutically acceptable salt thereof, wherein R₁ is selected from the group consisting of

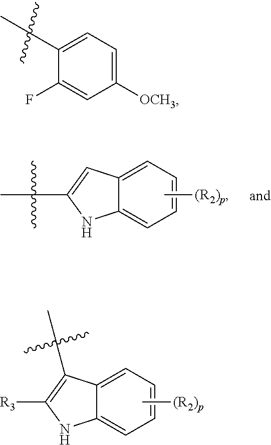

wherein
R₂ are individually the same or different and are selected from the group consisting of—OCH₃, —Cl, —OH, —CH₃, and —F;
R₃ are individually the same or different and are selected from the group consisting of—H or —CH₃;
n is 0-2;
R₄ are individually the same or different and are selected from —H or —CH₃;
R₅ is selected from the group consisting of:

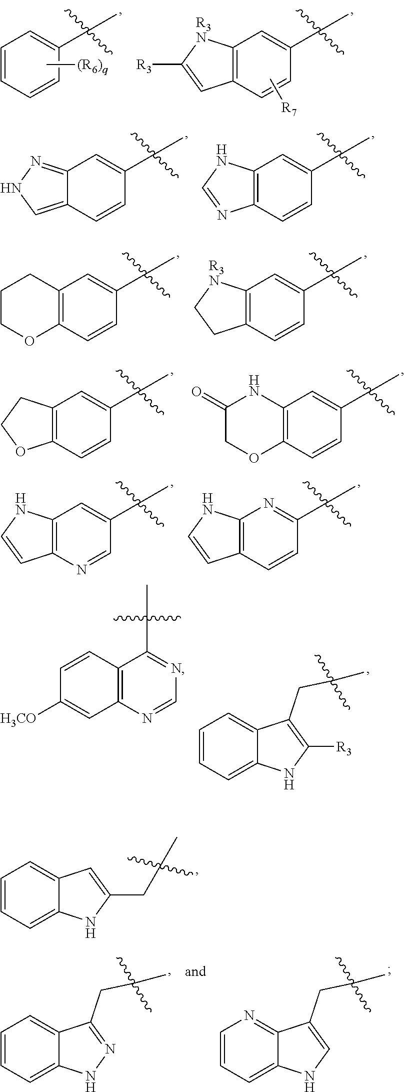

wherein
q is 0-3;
R₆ are independently the same or different and are selected from the group consisting of—F, —OCH₃, —OCHF₂, —SCH₃, —CH₃, —OCF₃, —OCH₂Ph, —OCH(CH₃)₂, —NHC(O)CH₃, —Cl, —OH, —N(CH₃)₂, —OEt, —CH₂CH₂OH, —S(O)₂CH₃,

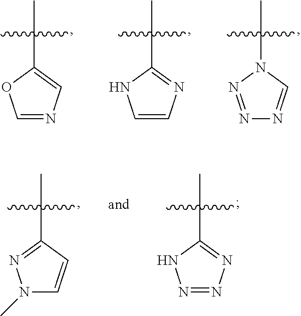

### R₇ is —H, —F or —Cl.

In further embodiments R₁ is

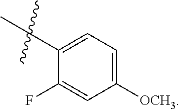 [SMILES: *c1ccc(OC)cc1F]

In further embodiments R₅ is selected from the group consisting of

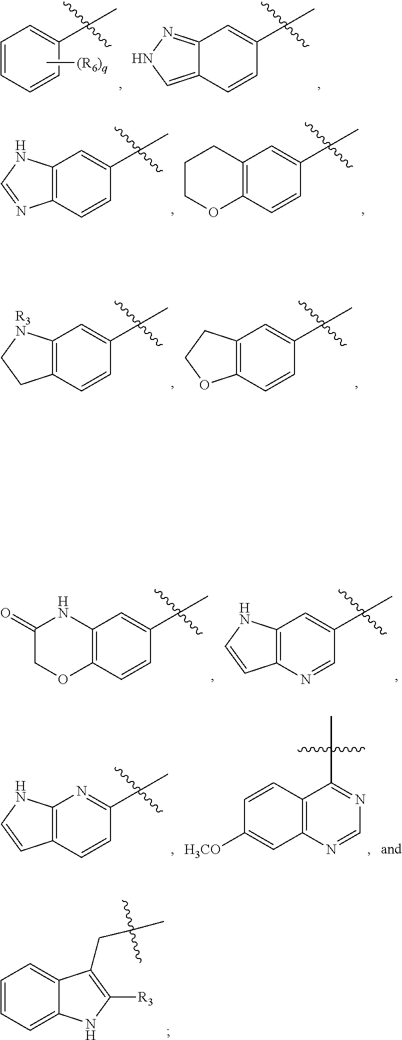

wherein
q is 0-3; R₆ are independently the same or different and are selected from the group consisting of—F, —OCH₃, —OCHF₂, —SCH₃, —CH₃, —OCF₃, —OCH₂Ph, —OCH(CH₃)₂, —Cl, —OH, —N(CH₃)₂, —OEt, —CH₂CH₂OH,

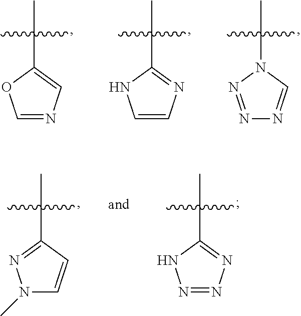

R₆ may be —OCH₃; and R₇ is —H, —F or —Cl.

Still further embodiments provide compounds given by Formula II or Formula III

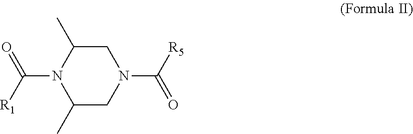

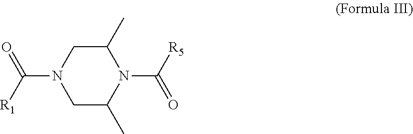

or a pharmaceutically acceptable salt thereof, wherein R₅ is selected from the group consisting of:

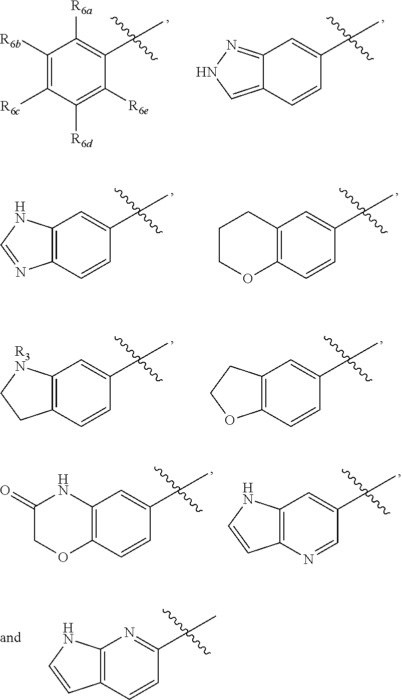

Wherein R₆ₐ is selected from the group consisting of—H, and —F; R_{6b} is selected from the group consisting of—H, and —F; R_{6c} is selected from the group consisting of—EtOH, —OCH₃, —Cl, —N(CH)₂, —OH, —F, —OCHF₂, —CH₃, —OCH(CH₃)₂, —OCF₃, —OCH₂Ph, —OEt, —SCH₃,

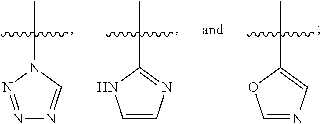 [SMILES: *n1cnnn1 ; *c1ncc[nH]1 ; *c1cnco1]

R_{6d} is selected from the group consisting of—H, and —F; R₆ₑ is selected from the group consisting of—H, and —F; wherein at least two of R₆ₐ, R_{6b}, R₆ₐ or R₆ₑ are —H.

Further embodiments provide a compound or pharmaceutically acceptable salt wherein R₅ is

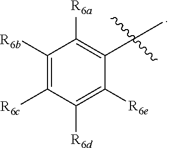

Further embodiments provide a compound or pharmaceutically acceptable salt wherein R₁ is selected from the group consisting of

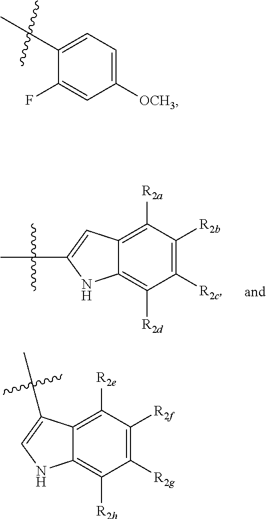

### Wherein

R₂ₐ is —H or —OCH₃;

### R_{2b} is —H or —OH;

### R_{2c}, is —H;

R_{2d} is —H or —OCH₃;

### R₂ₑ is —H;

### R_{2f} is —H, —F or —Cl;

### R_{2g} is —H; and

R₂ₕ is —H, —F, —Cl or —CH₃.
Further embodiments provide a compound or pharmaceutically acceptable salt wherein R₁ is

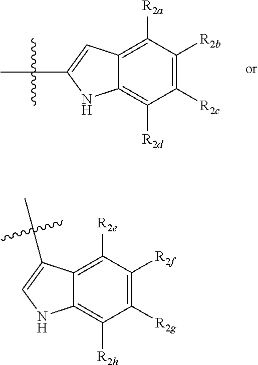

and wherein R₅ is selected from the group consisting of

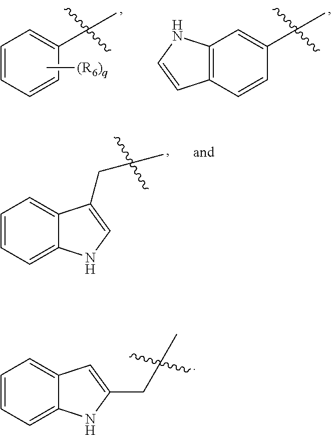

Further embodiments provide a compound or pharmaceutically acceptable salt wherein R₅ is selected from the group consisting of

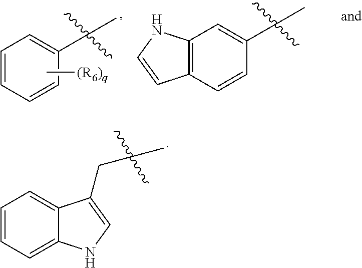

Further embodiments provide a compound or pharmaceutically acceptable salt wherein R₅ is

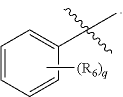

Further embodiments provide a compound or pharmaceutically acceptable salt wherein R₅ is

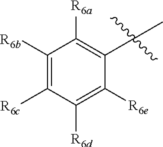

wherein
R₆ₐ is selected from the group consisting of—H, and —F;
R_{6b} is selected from the group consisting of—H, and —F;
R_{6c} is selected from the group consisting of—EtOH, —OCH₃, —Cl, —N(CH₃)₂, —OH, —F, —OCHF₂, —CH₃, —OCH(CH₃)₂, —OCF₃, —OCH₂Ph, —OEt, —SCH₃,

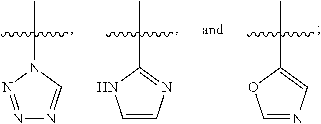 [SMILES: *n1cnnn1 ; *c1ncc[nH]1 ; *c1cnco1]

R₆ₐ is selected from the group consisting of—H, and —F;
R₆ₑ is selected from the group consisting of—H, and —F;
wherein at least two of R₆ₐ, R_{6b}, R_{6d} or R₆ₑ are —H.

Further embodiments may provide a pharmaceutical composition comprising an effective amount of a compound or pharmaceutically acceptable salt as reported herein and at least one pharmaceutically acceptable excipient.

Further embodiments provide a method of treating cancer in a subject in need of treatment, comprising administering to said subject an effective amount of a pharmaceutical composition, a compound, or a pharmaceutically acceptable salt reported herein.

Further embodiments provide a method of treating cancer in a subject in need of treatment, comprising administering to said subject an effective amount of a compound, pharmaceutically acceptable salt, or pharmaceutical composition as reported herein.

Further embodiments provide a method of treating cancer in a subject in need thereof, comprising: detecting an altered CPS1 status in a biological sample from said subject containing cells of said cancer, and if said cancer has said altered CPS1 status, administering to said subject an effective amount of a compound, pharmaceutically acceptable salt, or pharmaceutical composition as reported herein. In further embodiments “altered CPS1 status” is increased expression of CPS1.

Further embodiments provide use of a compound or pharmaceutically acceptable salt as reported herein in the manufacture of a medicament for treatment of cancer. Further embodiments provide use of a compound or pharmaceutically acceptable salt as reported herein.

Compounds of various embodiments as reported herein are shown in Table 1.

TABLE 1

| Compound | | |
|---|---|---|
| Number | STRUCTURE | Name |
| 1 | 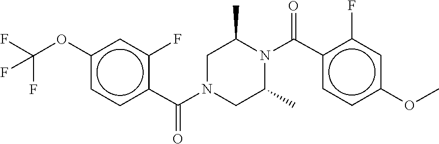 | ((2R,6R)-4-(2-fluoro-4- (trifluoromethoxy)benzoyl)- 2,6-dimethylpiperazin-1-yl)(2- fluoro-4- methoxyphenyl)methanone |
| 2 | 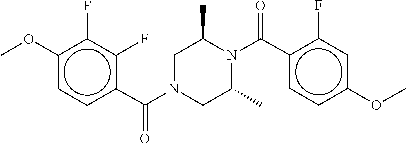 | ((2R,6R)-4-(2,3-difluoro-4- methoxybenzoyl)-2,6- dimethylpiperazin-1-yl)(2- fluoro-4- methoxyphenyl)methanone |
| 3 | 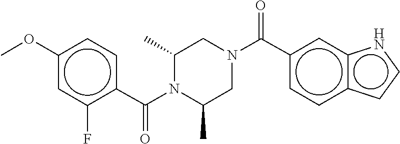 | ((2R,6R)-4-(1H-indole-6- carbonyl)-2,6- dimethylpiperazin-1-yl)(2- fluoro-4- methoxyphenyl)methanone |
| 4 | 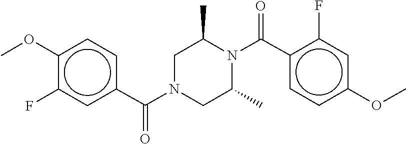 | ((2R,6R)-4-(3-fluoro-4- methoxybenzoyl)-2,6- dimethylpiperazin-1-yl)(2- fluoro-4- methoxyphenyl)methanone |
| 5 | 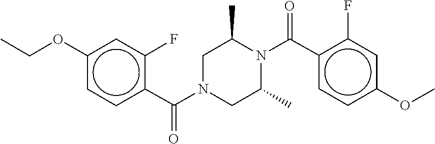 | ((2R,6R)-4-(4-ethoxy-2- fluorobenzoyl)-2,6- dimethylpiperazin-1-yl)(2- fluoro-4- methoxyphenyl)methanone |
| 6 | 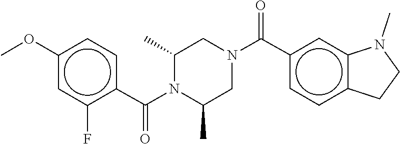 | ((2R,6R)-2,6-dimethyl-4-(1- methylindoline-6- carbonyl)piperazin-1-yl)(2- fluoro-4- methoxyphenyl)methanone |
| 7 | 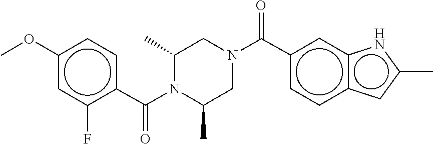 | ((2R,6R)-2,6-dimethyl-4-(2- methyl-1H-indole-6- carbonyl)piperazin-1-yl)(2- fluoro-4- methoxyphenyl)methanone |
| 8 | 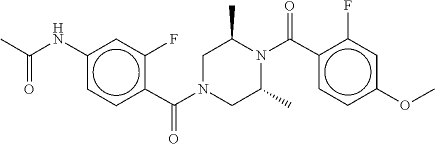 | N-(3-fluoro-4-((3R,5R)-4-(2- fluoro-4-methoxybenzoyl)- 3,5-dimethylpiperazine-1- carbonyl)phenyl)acetamide |
| 9 | 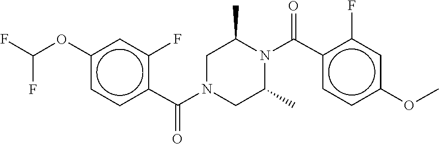 | ((2R,6R)-4-(4- (difluoromethoxy)-2- fluorobenzoyl)-2,6- dimethylpiperazin-1-yl)(2- fluoro-4- methoxyphenyl)methanone |
| 10 | 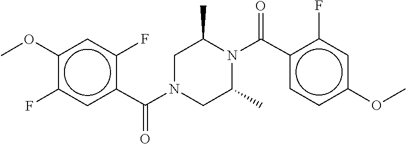 | ((2R,6R)-4-(2,5-difluoro-4- methoxybenzoyl)-2,6- dimethylpiperazin-1-yl)(2- fluoro-4- methoxyphenyl)methanone |
| 11 | 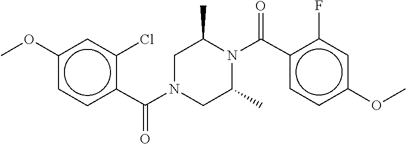 | ((2R,6R)-4-(2-chloro-4- methoxybenzoyl)-2,6- dimethylpiperazin-1-yl)(2- fluoro-4- methoxyphenyl)methanone |
| 12 | 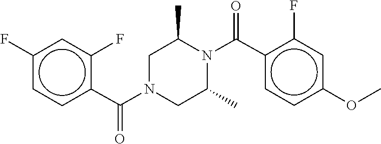 | ((2R,6R)-4-(2,4- difluorobenzoyl)-2,6- dimethylpiperazin-1-yl)(2- fluoro-4- methoxyphenyl)methanone |
| 13 | 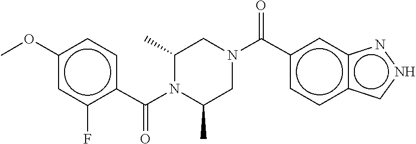 | ((2R,6R)-4-(2H-indazole-6- carbonyl)-2,6- dimethylpiperazin-1-yl)(2- fluoro-4- methoxyphenyl)methanone |
| 14 | 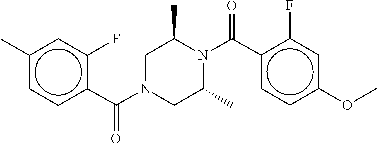 | ((3R,5R)-4-(2-fluoro-4- methoxybenzoyl)-3,5- dimethylpiperazin-1-yl)(2- fluoro-4- methylphenyl)methanone |
| 15 | 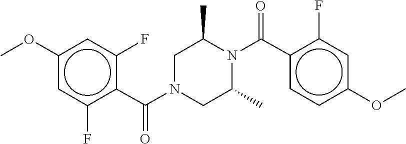 | ((2R,6R)-4-(2,6-difluoro-4- methoxybenzoyl)-2,6- dimethylpiperazin-1-yl)(2- fluoro-4- methoxyphenyl)methanone |
| 16 | 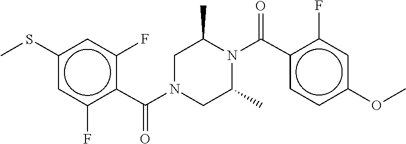 | ((2R,6R)-4-(2,6-difluoro-4- (methylthio)benzoyl)-2,6- dimethylpiperazin-1-yl)(2- fluoro-4- methoxyphenyl)methanone |
| 17 | 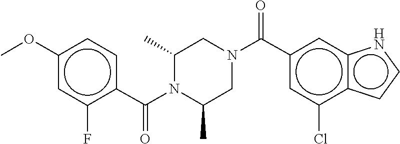 | (4-chloro-1H-indol-6- yl)((3R,5R)-4-(2-fluoro-4- methoxybenzoyl)-3,5- dimethylpiperazin-1- yl)methanone |
| 18 | 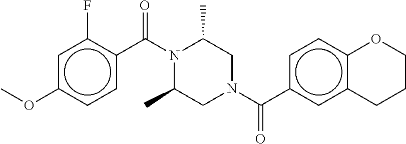 | ((2R,6R)-4-(chroman-6- carbonyl)-2,6- dimethylpiperazin-1-yl)(2- fluoro-4- methoxyphenyl)methanone |
| 19 | 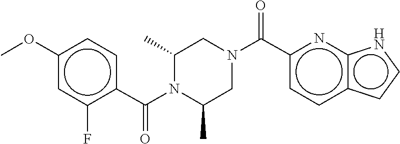 | ((2R,6R)-dimethyl-4-(1H- pyrrolo[2,3-b]pyridine-6- carbonyl)piperazin-1-yl)(2- fluoro-4- methoxyphenyl)methanone |
| 20 | 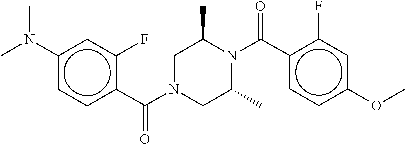 | ((2R,6R)-4-(4- (dimethylamino)-2- fluorobenzoyl)-2,6- dimethylpiperazin-1-yl)(2- fluoro-4- methoxyphenyl)methanone |
| 21 | 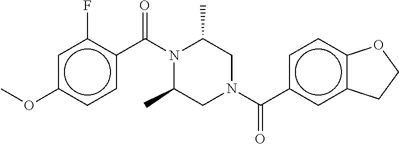 | ((2R,6R)-4-(2,3- dihydrobenzofuran-5- carbonyl)-2,6- dimethylpiperazin-1-yl)(2- fluoro-4- methoxyphenyl)methanone |
| 22 | 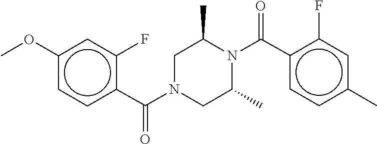 | ((2R,6R)-4-(2-fluoro-4- methoxybenzoyl)-2,6- dimethylpiperazin-1-yl)(2- fluoro-4- methylphenyl)methanone |
| 23 | 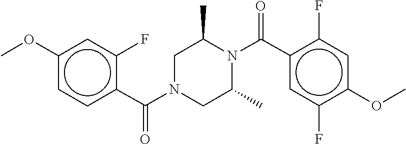 | ((3R,5R)-4-(2,5-difluoro-4- methoxybenzoyl)-3,5- dimethylpiperazin-1-yl)(2- fluoro-4- methoxyphenyl)methanone |
| 24 | 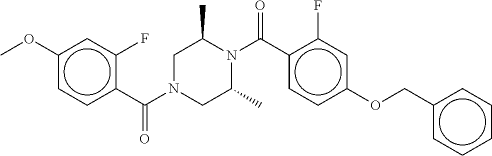 | ((3R,5R)-4-(4-(benzyloxy)-2- fluorobenzoyl)-3,5- dimethylpiperazin-1-yl)(2- fluoro-4- methoxyphenyl)methanone |
| 25 | 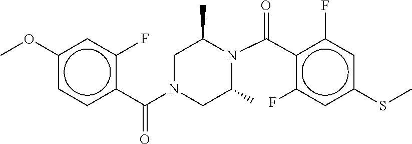 | ((3R,5R)-4-(2,6-difluoro-4- (methylthio)benzoyl)-3,5- dimethylpiperazin-1-yl)(2- fluoro-4- methoxyphenyl)methanone |
| 26 | 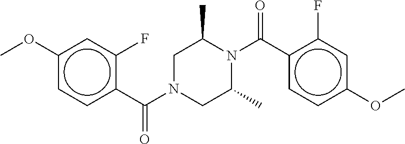 | ((2R,6R)-2,6- dimethylpiperazine-1,4- diyl)bis((2-fluoro-4- methoxyphenyl)methanone) |
| 27 | 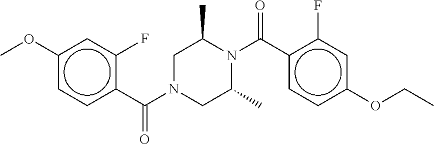 | ((3R,5R)-4-(4-ethoxy-2- fluorobenzoyl)-3,5- dimethylpiperazin-1-yl)(2- fluoro-4- methoxyphenyl)methanone |
| 28 | 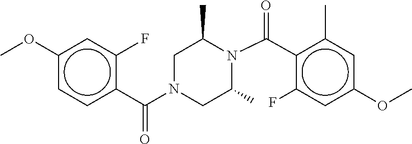 | ((3R,5R)-4-(2-fluoro-4- methoxy-6-methylbenzoyl)- 3,5-dimethylpiperazin-1-yl)(2- fluoro-4- methoxyphenyl)methanone |
| 29 | 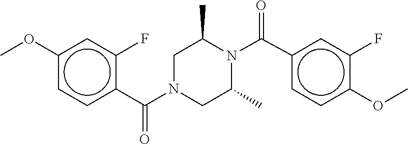 | ((2R,6R)-4-(2-fluoro-4- methoxybenzoyl)-2,6- dimethylpiperazin-1-yl)(3- fluoro-4- methoxyphenyl)methanone |
| 30 | 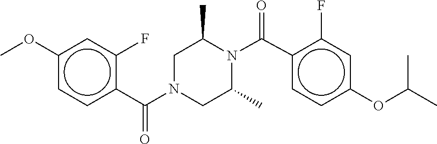 | ((3R,5R)-4-(2-fluoro-4- isopropoxybenzoyl)-3,5- dimethylpiperazin-1-yl)(2- fluoro-4- methoxyphenyl)methanone |
| 31 | 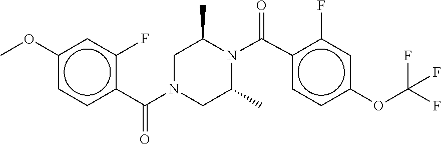 | ((3R,5R)-4-(2-fluoro-4- (trifluoromethoxy)benzoyl)- 3,5-dimethylpiperazin-1-yl)(2- fluoro-4- methoxyphenyl)methanone |
| 32 | 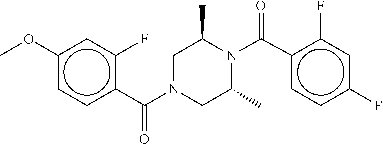 | ((3R,5R)-4-(2,4- difluorobenzoyl)-3,5- dimethylpiperazin-1-yl)(2- fluoro-4- methoxyphenyl)methanone |
| 33 | 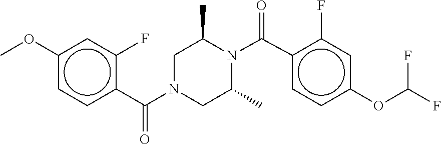 | ((3R,5R)-4-(4- (difluoromethoxy)-2- fluorobenzoyl)-3,5- dimethylpiperazin-1-yl)(2- fluoro-4- methoxyphenyl)methanone |
| 34 | 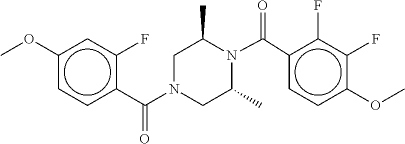 | ((3R,5R)-4-(2,3-difluoro-4- methoxybenzoyl)-3,5- dimethylpiperazin-1-yl)(2- fluoro-4- methoxyphenyl)methanone |
| 35 | 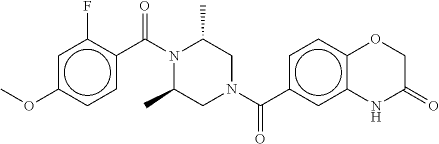 | 6-((3R,5R)-4-(2-fluoro-4- methoxybenzoyl)-3,5- dimethylpiperazine-1- carbonyl)-2H- benzo[b][1,4]oxazin-3(4H)- one |
| 36 | 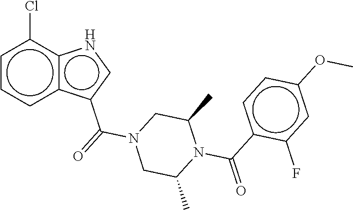 | (7-chloro-1H-indol-3-yl) 3R,5R)-4-(2-fluoro-4- methoxybenzoyl)-3,5- dimethylpiperazin-1- yl)methanone |
| 37 | 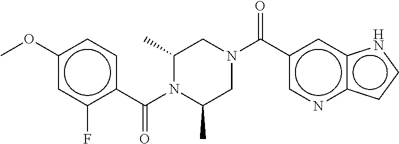 | ((2R,6R)-2,6-dimethyl-4-(1H- pyrrolo[3,2-b]pyridine-6- carbonyl)piperazin-1-yl)(2- fluoro-4- methoxyphenyl)methanone |
| 38 | 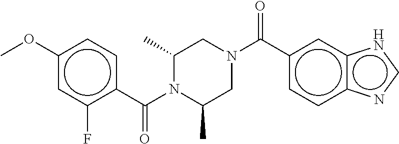 | ((2R,6R)-4-(1H- benzo[d]imidazole-6- carbonyl)-2,6- dimethylpiperazin-1-yl)(2- fluoro-4- methoxyphenyl)methanone |
| 39 | 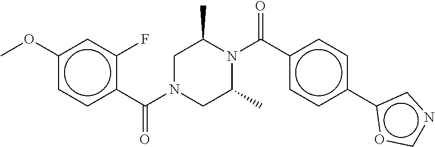 | ((3R,5R)-3,5-dimethyl-4-(4- (oxazol-5- yl)benzoyl)piperazin-1-yl)(2- fluoro-4- methoxyphenyl)methanone |
| 40 | 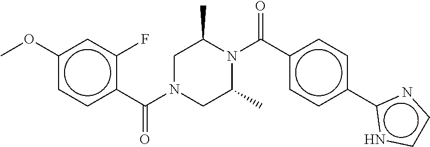 | ((3R,5R)-4-(4-(1H-imidazol-2- yl)benzoyl)-3,5- dimethylpiperazin-1-yl)(2- fluoro-4- methoxyphenyl)methanone |
| 41 | 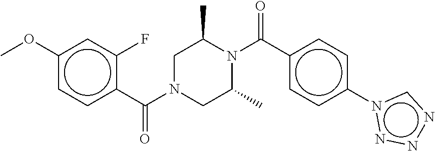 | ((3R,5R)-4-(4-(1H-tetrazol-1- yl)benzoyl)-3,5- dimethylpiperazin-1-yl)(2- fluoro-4- methoxyphenyl)methanone |
| 42 | 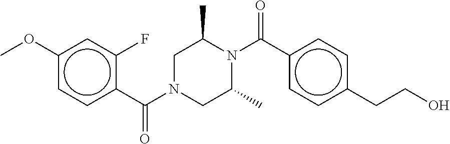 | ((2R,6R)-4-(2-fluoro-4- methoxybenzoyl)-2,6- dimethylpiperazin-1-yl)(4-(2- hydroxyethyl)phenyl) methanone |
| 43 | 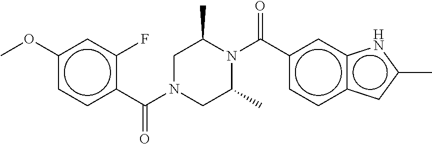 | ((3R,5R)-3,5-dimethyl-4-(2- methyl-1H-indole-6- carbonyl)piperazin-1-yl)(2- fluoro-4- methoxyphenyl)methanone |
| 44 | 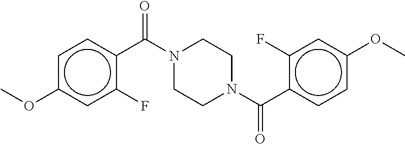 | piperazine-1,4-diylbis((2- fluoro-4- methoxyphenyl)methanone) |
| 45 | 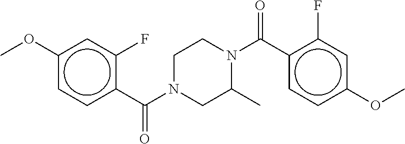 | (2-methylpiperazine-1,4- diyl)bis((2-fluoro-4- methoxyphenyl)methanone) |
| 46 | 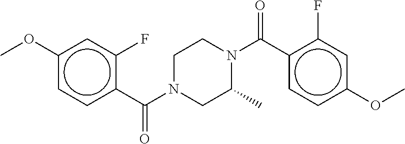 | (R)-(2-methylpiperazine-1,4- diyl)bis((2-fluoro-4- methoxyphenyl)methanone |
| 47 | 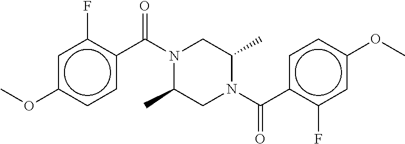 | ((2S,5R)-2,5- dimethylpiperazine-1,4- diyl)bis((2-fluoro-4- methoxyphenyl)methanone) |
| 48 | 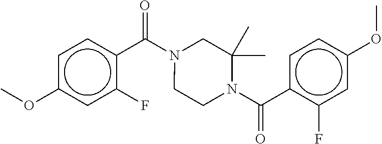 | (2,2-dimethylpiperazine-1,4- diyl)bis((2-fluoro-4- methoxyphenyl)methanone) |
| 49 | 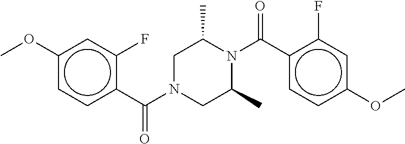 | ((2S,6S)-2,6- dimethylpiperazine-1,4- diyl)bis((2-fluoro-4- methoxyphenyl)methanone) |
| 50 | 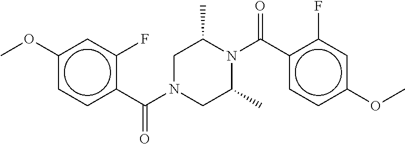 | ((2S,6R)-2,6- dimethylpiperazine-1,4- diyl)bis((2-fluoro-4- methoxyphenyl)methanone) |
| 51 | 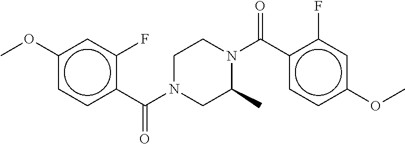 | (S)-(2-methylpiperazine-1,4- diyl)bis((2-fluoro-4- methoxyphenyl)methanone) |
| 52 | 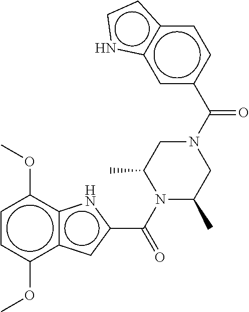 | ((2R,6R)-4-(1H-indole-6- carbonyl)-2,6- dimethylpiperazin-1-yl)(4,7- dimethoxy-1H-indol-2- yl)methanone |
| 53 | 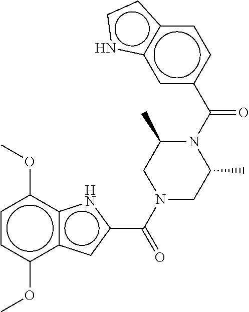 | ((3R,5R)-4-(1H-indole-6- carbonyl)-3,5- dimethylpiperazin-1-yl)(4,7- dimethoxy-1H-indol-2- yl)methanone |
| 54 | 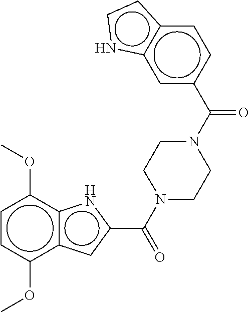 | (4-(1H-indole-6- carbonyl)piperazin-1-yl)(4,7- dimethoxy-1H-indol-2- yl)methanone |
| 55 | 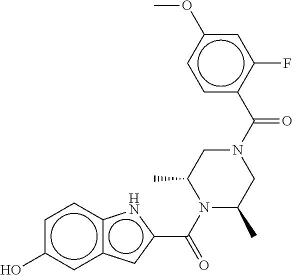 | ((2R,6R)-4-(2-fluoro-4- methoxybenzoyl)-2,6- dimethylpiperazin-1-yl)(5- hydroxy-1H-indol-2- yl)methanone |
| 56 | 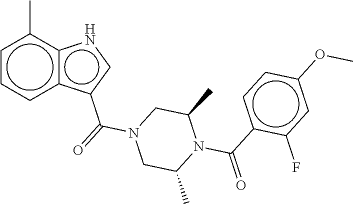 | ((2R,6R)-2,6-dimethyl-4-(7- methyl-1H-indole-3- carbonyl)piperazin-1-yl)(2- fluoro-4- methoxyphenyl)methanone |
| 57 | 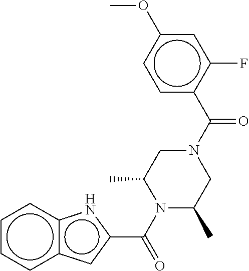 | ((3R,5R)-4-(1H-indole-2- carbonyl)-3,5- dimethylpiperazin-1-yl)(2- fluoro-4- methoxyphenyl)methanone |
| 58 | 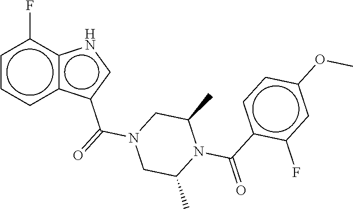 | (7-fluoro-1H-indol-3- yl)((3R,5R)-4-(2-fluoro-4- methoxybenzoyl)-3,5- dimethylpiperazin-1- yl)methanone |
| 59 | 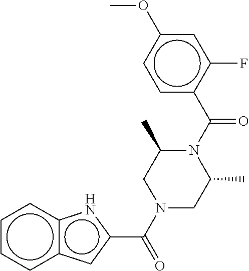 | ((2R,6R)-4-(1H-indole-2- carbonyl)-2,6- dimethylpiperazin-1-yl)(2- fluoro-4- methoxyphenyl)methanone |
| 60 | 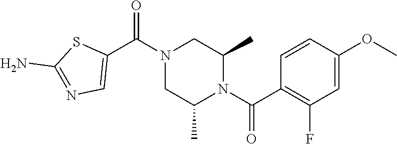 [SMILES: COc1ccc(C(=O)N2[C@H](C)CN(C(=O)c3cnc(N)s3)C[C@H]2C)c(F)c1] | (2-aminothiazolidin-5- yl)((3R,5R)-4-(2-fluoro-4- methoxybenzoyl)-3,5- dimethylpiperazin-1- yl)methanone |
| 61 | 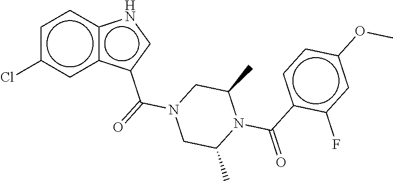 | (5-chloro-1H-indol-3- yl)((3R,5R)-4-(2-fluoro-4- methoxybenzoyl)-3,5- dimethylpiperazin-1- yl)methanone |
| 62 | 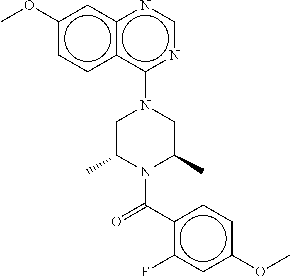 | (2-fluoro-4- methoxyphenyl)((2R,6R)-4-(7- methoxyquinazolin-4-yl)-2,6- dimethylpiperazin-1- yl)methanone |
| 63 | 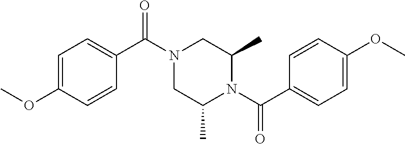 | ((2R,6R)-2,6- dimethylpiperazine-1,4- diyl)bis((4- methoxyphenyl)methanone) |
| 64 | 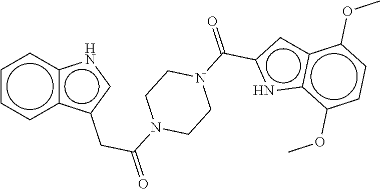 | 1-(4-(4,7-dimethoxy-1H- indole-2-carbonyl)piperazin-1- yl)-2-(1H-indol-3-yl)ethanone |
| 65 | 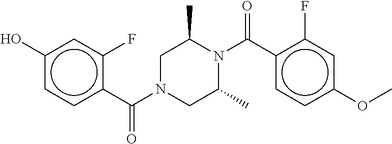 | ((2R,6R)-4-(2-fluoro-4- hydroxybenzoyl)-2,6- dimethylpiperzin-1-yl)(2- fluoro-4- methoxyphenyl)methanone |
| 66 | 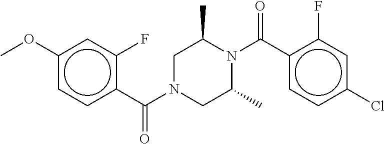 | ((3R,5R)-4-(4-chloro-2- fluorobenzoyl)-3,5- dimethylpiperazin-1-yl)(2- fluoro-4- methoxyphenyl)methanone |
| 67 | 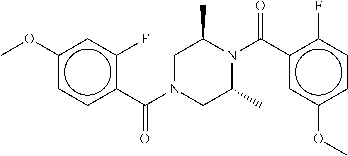 | ((2R,6R)-4-(2-fluoro-4- methoxybenzoyl)-2,6- dimethylpiperazin-1-yl)(2- fluoro-5- methoxyphenyl)methanone |
| 68 | 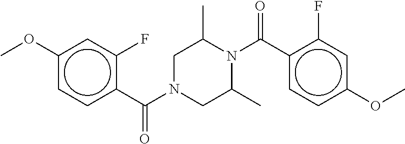 | (2,6-dimethylpiperazine-1,4- diyl)bis((2-fluoro-4- methoxyphenyl)methanone) |
| 69 | 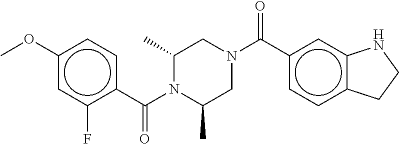 | ((3R,5R)-4-(2-fluoro-4- methoxybenzoyl)-3,5- dimethylpiperazin-1- yl)(indolin-6-yl)methanone |
| 70 | 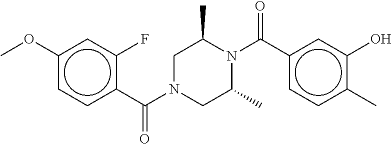 | ((2R,6R)-4-(2-fluoro-4- methoxybenzoyl)-2,6- dimethylpiperazin-1-yl)(3- hydroxy-4- methylphenyl)methanone) |
| 71 | 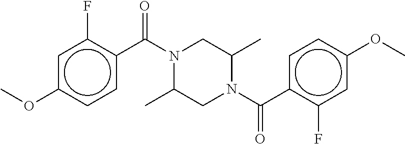 | (2,5-dimethylpiperazine-1,4- diyl)bis((2-fluoro-4- methoxyphenyl)methanone |
| 72 | 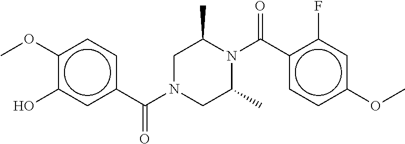 | ((3R,5R)-4-(2-fluoro-4- methoxybenzoyl)-3,5- dimethylpiperazin-1-yl)(3- hydroxy-4- methoxyphenyl)methanone |
| 73 | 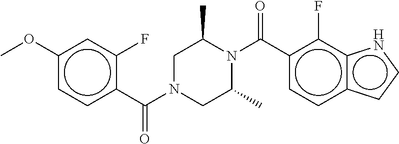 | (7-fluoro-1H-indol-6- yl)((2R,6R)-4-(2-fluoro-4- methoxybenzoyl)-2,6- dimethylpiperazin-1- yl)methanone |
| 74 | 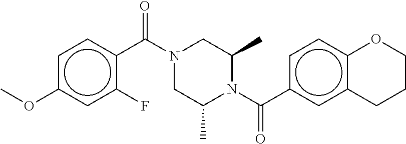 | ((3R,5R)-4-(chroman-6- carbonyl)-3,5- dimethylpiperazin-1-yl)(2- fluoro-4- methoxyphenyl)methanone |
| 75 | 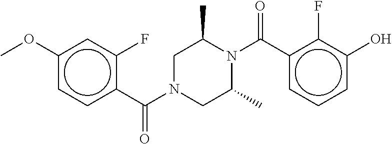 | ((3R,5R)-4-(2-fluoro-3- hydroxybenzoyl)-3,5- dimethylpiperazin-1-yl)(2- fluoro-4- methoxyphenyl)methanone |
| 76 | 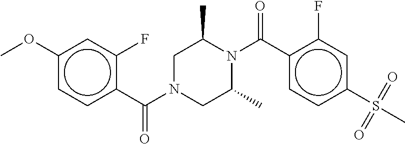 | ((3R,5R)-4-(2-fluoro-4- (methylsulfonyl)benzoyl)-3,5- dimethylpiperazin-1-yl)(2- fluoro-4- methoxyphenyl)methanone |
| 77 | 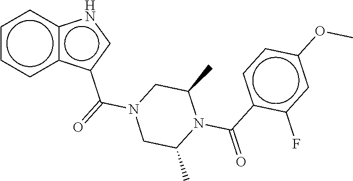 | ((2R,6R)-4-(1H-indole-3- carbonyl)-2,6- dimethylpiperazin-1-yl)(2- fluoro-4- methoxyphenyl)methanone |
| 78 | 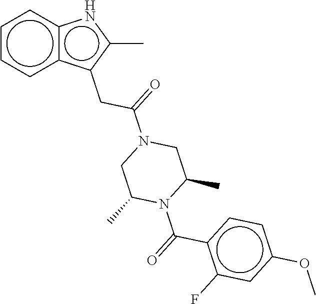 | 1-((3R,5R)-4-(2-fluoro-4- methoxybenzoyl)-3,5- dimethylpiperazin-1-yl)-2-(2- methyl-1H-indol-3- yl)ethanone |
| 79 | 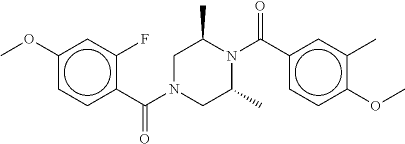 | ((2R,6R)-4-(2-fluoro-4- methoxybenzoyl)-2,6- dimethylpiperazin-1-yl)(4- methoxy-3- methylphenyl)methanone |
| 80 | 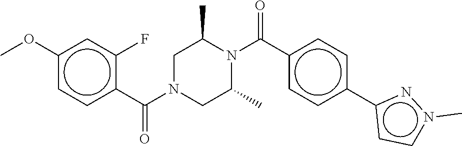 | ((3R,5R)-3,5-dimethyl-4-(4- (1-methyl-1H-pyrazol-3- yl)benzoyl)piperazin-1-yl)(2- fluoro-4- methoxyphenyl)methanone |
| 81 | 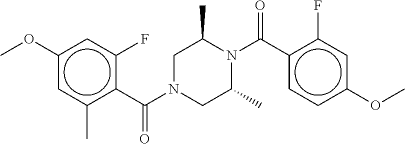 | ((2R,6R)-4-(2-fluoro-4- methoxy-6-methylbenzoyl)- 2,6-dimethylpiperazin-1-yl)(2- fluoro-4- methoxyphenyl)methanone |
| 82 | 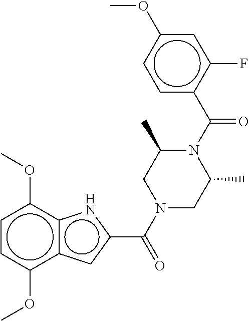 | (4,7-dimethoxy-1H-indol-2- yl)((3R,5R)-4-(2-fluoro-4- methoxybenzoyl)-3,5- dimethylpiperazin-1- yl)methanone |
| 83 | 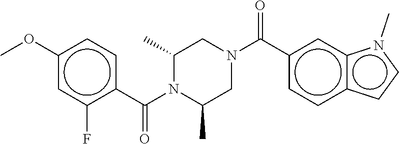 | ((2R,6R)-2,6-dimethyl-4-(1- methyl-1H-indole-6- carbonyl)piperazin-1-yl)(2- fluoro-4- methoxyphenyl)methanone |
| 84 | 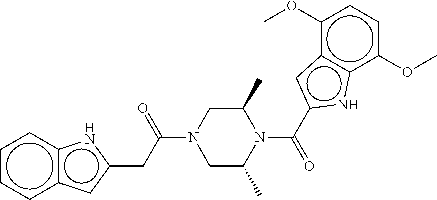 | 1-((3R,5R)-4-(4,7-dimethoxy- 1H-indole-2-carbonyl)-3,5- dimethylpiperazin-1-yl)-2- (1H-indol-2-yl)ethanone |
| 85 | 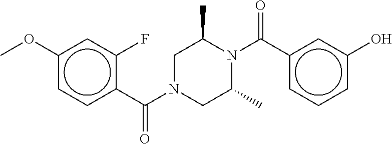 | ((2R,6R)-4-(2-fluoro-4- methoxybenzoyl)-2,6- dimethylpiperazin-1-yl)(3- hydroxyphenyl)methanone |
| 86 | 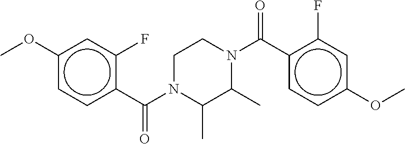 | (2,3-dimethylpiperazine-1,4- diyl)bis((2-fluoro-4- methoxyphenyl)methanone) |
| 87 | 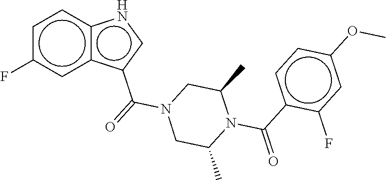 | (5-fluoro-1H-indol-3- yl)((3R,5R)-4-(2-fluoro-4- methoxybenzoyl)-3,5- dimethylpiperazin-1- yl)methanone |
| 88 | 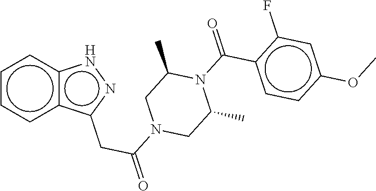 | 1-((3R,5R)-4-(2-fluoro-4- methoxybenzoyl)-3,5- dimethylpiperazin-1-yl)-2- (1H-indazol-3-yl)ethanone |
| 89 | 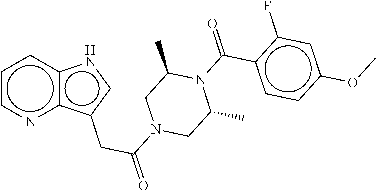 | 1-((3R,5R)-4-(2-fluoro-4- methoxybenzoyl)-3,5- dimethylpiperazin-1-yl)-2- (1H-pyrrolo[3,2-b]pyridin-3- yl)ethanone |
| 90 | 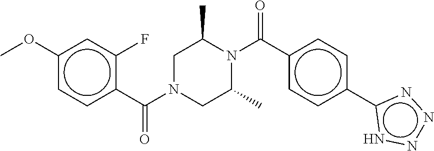 | ((3R,5R)-4-(4-(1H-tetrazol-5- yl)benzoyl)-3,5- dimethylpiperazin-1-yl)(2- fluoro-4- methoxyphenyl)methanone |
| 91 | 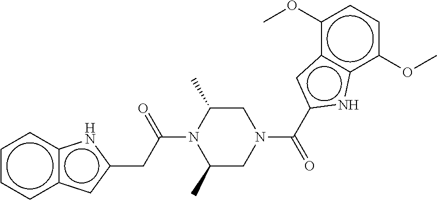 | 1-((2R,6R)-4-(4,7-dimethoxy- 1H-indole-2-carbonyl)-2,6- dimethylpiperazin-1-yl)-2- (1H-indol-2-yl)ethanone |
| 92 | 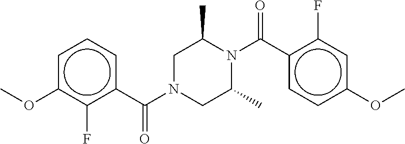 | ((2R,6R)-4-(2-fluoro-3- methoxybenzoyl)-2,6- dimethylpiperazin-1-yl)(2- fluoro-4- methoxyphenyl)methanone |
| 93 | 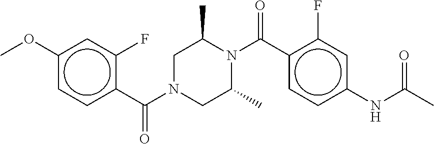 | N-(3-fluoro-4-((2R,6R)-4-(2- fluoro-4-methoxybenzoyl)- 2,6-dimethylpiperazine-1- carbonyl)phenyl)acetamide |
| 94 | 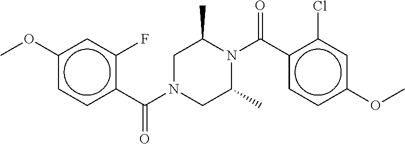 | ((3R,5R)-4-(2-chloro-4- methoxybenzoyl)-3,5- dimethylpiperazin-1-yl)(2- fluoro-4- methoxyphenyl)methanone |
| 95 | 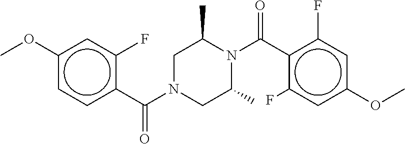 | ((3R,5R)-4-(2,6-difluoro-4- methoxybenzoyl)-3,5- dimethylpiperazin-1-yl)(2- fluoro-4- methoxyphenyl)methanone |
| 96 | 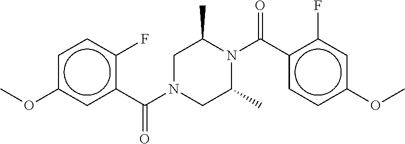 | ((3R,5R)-4-(2-fluoro-4- methoxybenzoyl)-3,5- dimethylpiperazin-1-yl)(2- fluoro-5- methoxyphenyl)methanone |
| 97 | 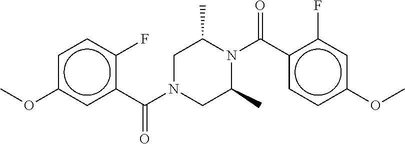 | ((3S,5S)-4-(2-fluoro-4- methoxybenzoyl)-3,5- dimethylpiperazin-1-yl)(2- fluoro-5- methoxyphenyl)methanone |
| 98 | 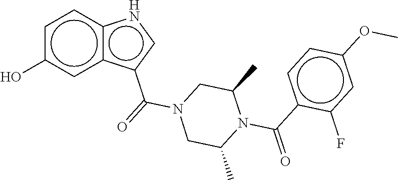 | ((3R,5R)-4-(2-fluoro-4- methoxybenzoyl)-3,5- dimethylpiperazin-1-yl)(5- hydroxy-1H-indol-3- yl)methanone |
| 99 | 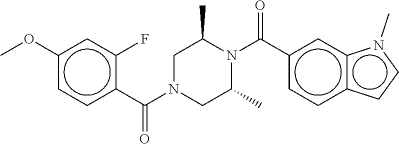 | ((3R,5R)-3,5-dimethyl-4-(1- methyl-1H-indole-6- carbonyl)piperazin-1-yl)(2- fluoro-4- methoxyphenyl)methanone |
| 100 | 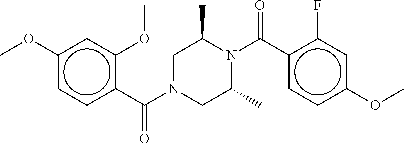 | ((2R,6R)-4-(2,4- dimethoxybenzoyl)-2,6- dimethylpiperazin-1-yl)(2- fluoro-4- methoxyphenyl)methanone |
| 101 | 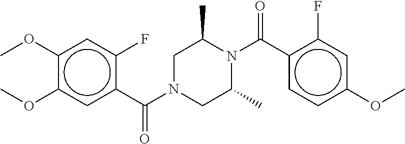 | ((2R,6R)-4-(2-fluoro-4,5- dimethoxybenzoyl)-2,6- dimethylpiperazin-1-yl)(2- fluoro-4- methoxyphenyl)methanone |
| 102 | 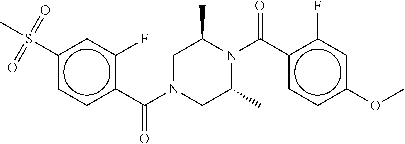 | ((2R,6R)-4-(2-fluoro-4- (methylsulfonyl)benzoyl)-2,6- dimethylpiperazin-1-yl)(2- fluoro-4- methoxyphenyl)methanone |
| 103 | 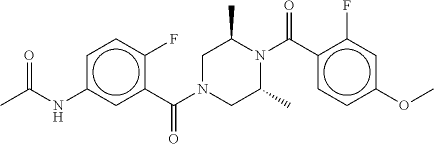 | N-(4-fluoro-3-((3R,5R)-4-(2- fluoro-4-methoxybenzoyl)- 3,5-dimethylpiperazine-1- carbonyl)phenyl)acetamide |
| 104 | 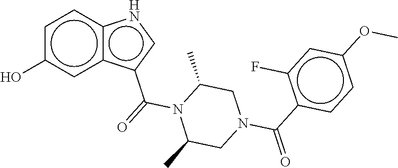 | ((2R,6R)-4-(2-fluoro-4- methoxybenzoyl)-2,6- dimethylpiperazin-1-yl)(5- hydroxy-1H-indol-3- yl)methanone |
| 105 | 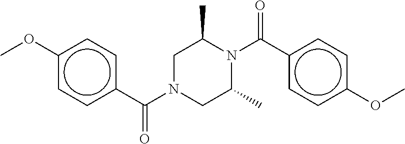 | ((2R,6R)-2,6- dimethylpiperazine-1,4- diyl)bis((4- methoxyphenyl)methanone) |
| 106 | 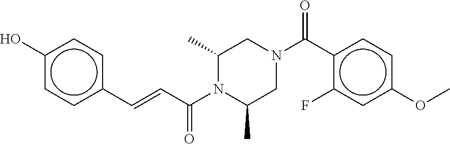 | (E)-1-((2R,6R)-4-(2-fluoro-4- methoxybenzoyl)-2,6- dimethylpiperazin-1-yl)-3-(4- hydroxypheny)prop-2-en-1- one |
| 107 | 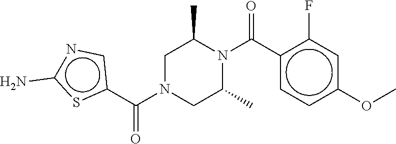 | (2-aminothiazol-5- yl)((3R,5R)-4-(2-fluoro-4- methoxybenzoyl)-3,5- dimethylpiperazin-1- yl)methanone |
| 108 | 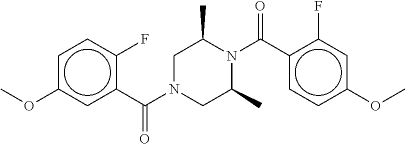 | ((3S,5R)-4-(2-fluoro-4- methoxybenzoyl)-3,5- dimethylpiperazin-1-yl)(2- fluoro-5- methoxyphenyl)methanone |

### DETAILED DESCRIPTION

All publications and patent documents cited herein are incorporated herein by reference as if each such publication or document was specifically and individually indicated to be incorporated herein by reference. Where the text of this disclosure and the text of one or more documents incorporated by reference conflicts, this disclosure controls. Citation of publications and patent documents is not intended as an admission that any is pertinent prior art, nor does it constitute any admission as to the contents or date of the same. The embodiments described herein having now been described by way of written description, those of skill in the art will recognize that the embodiments described herein may be practiced in a variety of embodiments and that the description and examples provided herein are for purposes of illustration and not limitation of the claims.

As used herein, “alkyl,” “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include moieties having from one to six carbon atoms, such as, but not limited to, methyl, ethyl, n-propyl, i-propyl, n-butyl, s-butyl, t-butyl, n-pentyl, s-pentyl or n-hexyl.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.
As used herein, the term “cycloalkyl” refers to a saturated or unsaturated nonaromatic hydrocarbon ring having 3 to 7 carbon atoms (e.g., C₃-C₇). Examples of cycloalkyl include, but are not limited to, cyclopropyl, cyclobutyl, cyclopentyl, cyclohexyl, cycloheptyl, cyclopentenyl, cyclohexenyl, and cycloheptenyl.

The term “heterocycloalkyl” refers to a saturated or unsaturated nonaromatic 3-8 membered monocyclic groups, 7-10 membered fused bicyclic groups having one or more heteroatoms (such as O, N, or S), unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, tetrahydrothiophene, dihydropyranyl, pyranyl, morpholinyl, 1,4-diazepanyl, 1,4-oxazepanyl, and the like.

Additional examples of heterocycloalkyl groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl.

The term “optionally substituted alkyl” refers to unsubstituted alkyl or alkyl having designated substituents replacing one or more hydrogen atoms on one or more carbons of the hydrocarbon backbone. Such substituents may include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

An “arylalkyl” or an “aralkyl” moiety is an alkyl substituted with an aryl (e.g., phenylmethyl(benzyl)). An “alkylaryl” moiety is an aryl substituted with an alkyl (e.g., methylphenyl).

“Alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

The term “optionally substituted alkenyl” refers to unsubstituted alkenyl or alkenyl having designated substituents replacing one or more hydrogen atoms on one or more hydrocarbon backbone carbon atoms. Such substituents may include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

The term “optionally substituted alkynyl” refers to unsubstituted alkynyl or alkynyl having designated substituents replacing one or more hydrogen atoms on one or more hydrocarbon backbone carbon atoms. Such substituents may include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

“Aryl” includes groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR′ wherein R′ is H or other substituents, as defined).

The nitrogen and sulfur heteroatoms may optionally be oxidized (i.e., N→O and S(O)ₚ,
where p=1 or 2). It is to be noted that total number of S and O atoms in the aromatic heterocycle is not more than 1.
Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., bicyclic. Non-limiting example of such aryl groups include, e.g., naphthalene, benzoxazole, benzodioxazole, benzothiazole, benzoimidazole, benzothiophene, methylenedioxyphenyl, quinoline, isoquinoline, naphthrydine, indole, benzofuran, purine, benzofuran, deazapurine, indolizine.

In the case of multicyclic aromatic rings, only one of the rings needs to be aromatic (e.g., 2,3-dihydroindole), although all of the rings may be aromatic (e.g., quinoline).

The cycloalkyl, heterocycloalkyl, aryl, or heteroaryl ring may be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups may also be fused with alicyclic or heterocyclic rings, which are not aromatic so as to form a multicyclic system (e.g., tetralin, methylenedioxyphenyl).

When a bond to a substituent is shown to cross a bond connecting two atoms in a ring (as shown by the examples below with substituent R), then such substituent may be bonded to any atom in the ring.

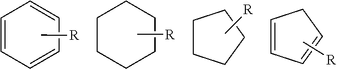

When any variable (e.g., R₁) occurs more than one time in any constituent or formula for a compound, its definition at each occurrence is independent of its definition at every other occurrence. Thus, for example, if a group is shown to be substituted with 0-2 R₁ moieties, then the group may optionally be substituted with up to two R₁ moieties and R₁ at each occurrence is selected independently from the definition of R₁.

The term “hydroxy” or “hydroxyl” includes groups with an —OH or —O.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms. “Alkoxyalkyl,” “alkylaminoalkyl,” and “thioalkoxyalkyl” include alkyl groups, as described above, wherein oxygen, nitrogen, or sulfur atoms replace one or more hydrocarbon backbone carbon atoms.

The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups may be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Calm et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Calm et al., Experientia 1956, 12, 81; Cahn, J. Chem. Educ. 1964, 41, 116).

In the present specification, each incidence of a chiral center within a structural formula, such as the non-limiting example shown here:

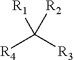

is meant to depict all possible stereoisomers. In contrast, a chiral center drawn with hatches and wedges, such as the non-limiting example shown here:

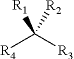

is meant to depict the stereoisomer as indicated (here in this sp³ hybridized carbon chiral center, R₃ and R₄ are in the plane of the paper, R₁ is above the plane of paper, and R₂ is behind the plane of paper).

“Geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

In the present specification, each incidence within a structural formula including a wavy line adjacent to a double bond as shown:

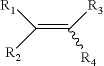

is meant to depict both geometric isomers. In contrast, such structures drawn without a wavy line is meant to depict a compound having the geometric configuration as drawn.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Where the present specification depicts a compound prone to tautomerization, but only depicts one of the tautomers, it is understood that all tautomers are included as part of the meaning of the chemical depicted. It is to be understood that the compounds disclosed herein may be depicted as different tautomers. It should also be understood that when compounds have tautomeric forms, all tautomeric forms are intended to be included, and the naming of the compounds does not exclude any tautomer form.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine.

Furthermore, the structures and other compounds disclosed herein include all atropic isomers thereof, it being understood that not all atropic isomers may have the same level of activity. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

The term “crystal polymorphs,” “polymorphs” or “crystal forms” means crystal structures in which a compound (or a salt or solvate thereof) may crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds may be prepared by crystallization under different conditions. It is understood that the compounds disclosed herein may exist in crystalline form, crystal form mixture, or anhydride or hydrate thereof.

The compounds disclosed herein include the compounds themselves, as well as their salts and solvates, if applicable. A salt, for example, may be formed between an anion and a positively charged group (e.g., amino) on an aryl- or heteroaryl-substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt may also be formed between a cation and a negatively charged group (e.g., carboxylate) on an aryl- or heteroaryl-substituted benzene compound. Suitable cations include sodium ion, potassium ion, magnesium ion, calcium ion, and an ammonium cation such as tetramethylammonium ion. The aryl- or heteroaryl-substituted benzene compounds also include those salts containing quaternary nitrogen atoms.

Additionally, the compounds disclosed herein, for example, the salts of the compounds, may exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, “pharmaceutically acceptable salts” refer to derivatives of the compounds disclosed herein wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt may be 1:1, or any ratio other than 1:1, e.g., 3:1, 2:1, 1:2, or 1:3.

It should be understood that all references to pharmaceutically acceptable salts include solvent addition forms (solvates) or crystal forms (polymorphs) as defined herein, of the same salt.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

Chemicals as named or depicted are intended to include all naturally occurring isotopes of atoms occurring in the present compounds. Isotopes include those atoms having the same atomic number but different mass numbers. By way of general example and without limitation, isotopes of ¹H hydrogen include tritium and deuterium, and isotopes of ¹²C carbon include ¹³C and ¹⁴C.

It will be understood that some compounds, and isomers, salts, esters and solvates thereof, of the compounds disclosed herein may exhibit greater in vivo or in vitro activity than others. It will also be appreciated that some cancers may be treated more effectively than others, and may be treated more effectively in certain species of subjects that others, using the compounds, and isomers, salts, esters and solvates thereof, of the compounds disclosed herein.

As used herein, “treating” means administering to a subject a pharmaceutical composition to ameliorate, reduce or lessen the symptoms of a disease. As used herein, “treating” or “treat” describes the management and care of a subject for the purpose of combating a disease, condition, or disorder and includes the administration of a compound disclosed herein, or a pharmaceutically acceptable salt, polymorph or solvate thereof, to alleviate the symptoms or complications of a disease, condition or disorder, or to eliminate the disease, condition or disorder. The term “treat” may also include treatment of a cell in vitro or an animal model.

Treating cancer may result in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

As used herein, “subject” or “subjects” refers to any animal, such as mammals including rodents (e.g., mice or rats), dogs, primates, lemurs or humans.

Treating cancer may result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in increase in average survival time of a population of treated subjects in comparison to a population receiving monotherapy with a drug that is not a compound disclosed herein, or a pharmaceutically acceptable salt thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in a decrease in the mortality rate of a population of treated subjects in comparison to a population receiving carrier alone. Treating cancer may result in a decrease in the mortality rate of a population of treated subjects in comparison to an untreated population. Treating cancer may result in a decrease in the mortality rate of a population of treated subjects in comparison to a population receiving monotherapy with a drug that is not a compound disclosed herein, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer may result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate may be measured according to a change in tumor diameter per unit time.

Treating cancer may result in a decrease in tumor regrowth, for example, following attempts to remove it surgically. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder may result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder may result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells may be equivalent to the mitotic index.

Treating or preventing a cell proliferative disorder may result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder may result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology may be measured by microscopy, e.g., using an inverted tissue culture microscope. An abnormal cellular morphology may take the form of nuclear pleiomorphism.

As used herein, the term “alleviate” is meant to describe a process by which the severity of a sign or symptom of a disorder is decreased. Importantly, a sign or symptom may be alleviated without being eliminated. In a preferred embodiment, the administration of pharmaceutical compositions disclosed herein leads to the elimination of a sign or symptom, however, elimination is not required. Effective dosages are expected to decrease the severity of a sign or symptom. For instance, a sign or symptom of a disorder such as cancer, which may occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the Amerimay Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of embodiments described herein, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by non-health-care professionals.

A “pharmaceutical composition” is a formulation containing a compound disclosed herein in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound disclosed herein include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The present disclosure also provides pharmaceutical compositions comprising any compound disclosed herein in combination with at least one pharmaceutically acceptable excipient or carrier.

A pharmaceutical composition disclosed herein is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH may be adjusted with acids or bases, such as hydrochloric acid or sodium hydroxide. The parenteral preparation may be enclosed in ampoules, disposable syringes or multiple dose vials made of glass or plastic.

A compound or pharmaceutical composition disclosed herein may be administered to a subject in many of the well-known methods currently used for chemotherapeutic treatment. For example, for treatment of cancers, a compound disclosed herein may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The term “therapeutically effective amount,” as used herein, refers to an amount of a pharmaceutical agent to treat, ameliorate, or prevent an identified disease or condition, or to exhibit a detectable therapeutic or inhibitory effect. The effect may be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation may be determined by routine experimentation that is within the skill and judgment of the clinician. In a preferred aspect, the disease or condition to be treated is cancer. In another aspect, the disease or condition to be treated is a cell proliferative disorder.

For any compound, the therapeutically effective amount may be estimated initially either in cell culture assays, e.g., of neoplastic cells, or in animal models, usually rats, mice, rabbits, dogs, or pigs. The animal model may also be used to determine the appropriate concentration range and route of administration. Such information may then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it may be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds disclosed herein may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that may be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier may be a solvent or dispersion medium containing, for example, water, ethanol, polyol (for example, glycerol, propylene glycol, and liquid polyethylene glycol, and the like), and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions may be brought about by including in the composition an agent which delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents, and/or adjuvant materials may be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The active compounds may be prepared with pharmaceutically acceptable carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the compounds disclosed herein are dictated by and directly dependent on the unique characteristics of the active compound and the particular therapeutic effect to be achieved.

In therapeutic applications, the dosages of the pharmaceutical compositions used in accordance with embodiments described herein vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages may range from about 0.01 mg/kg per day to about 5000 mg/kg per day. In preferred aspects, dosages may range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions may be included in a container, pack, or dispenser together with instructions for administration.

Techniques for formulation and administration of the compounds disclosed herein may be found in Remington: the Science and Practice of Pharmacy, 19^{th} edition, Mack Publishing Co., Easton, Pa. (1995). In an embodiment, the compounds described herein, and the pharmaceutically acceptable salts thereof, may be used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

Exemplary cancers that may be treated using one or more compounds disclosed herein include, but are not limited to, lung, glioblastoma, colon, prostate, bladder, esophageal and endometrial cancers.

A cancer that is to be treated may include a tumor that has been determined to be less than or equal to about 2 centimeters in diameter. A cancer that is to be treated may include a tumor that has been determined to be from about 2 to about 5 centimeters in diameter. A cancer that is to be treated may include a tumor that has been determined to be greater than or equal to about 3 centimeters in diameter. A cancer that is to be treated may include a tumor that has been determined to be greater than 5 centimeters in diameter. A cancer that is to be treated may be classified by microscopic appearance as well differentiated, moderately differentiated, poorly differentiated, or undifferentiated.

A cancer that is to be treated may be classified by microscopic appearance with respect to mitosis count (e.g., amount of cell division) or nuclear pleiomorphism (e.g., change in cells). A cancer that is to be treated may be classified by microscopic appearance as being associated with areas of necrosis (e.g., areas of dying or degenerating cells). A cancer that is to be treated may be classified as having an abnormal karyotype, having an abnormal number of chromosomes, or having one or more chromosomes that are abnormal in appearance. A cancer that is to be treated may be classified as being aneuploid, triploid, tetraploid, or as having an altered ploidy. A cancer that is to be treated may be classified as having a chromosomal translocation, or a deletion or duplication of an entire chromosome, or a region of deletion, duplication or amplification of a portion of a chromosome.

The compounds, or pharmaceutically acceptable salts thereof may be administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds may be selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian may readily determine and prescribe the effective amount of the drug required to prevent, counter, or arrest the progress of the condition.

### EXAMPLES

Table 2 presents non-limiting examples of embodiments of compounds disclosed herein, along with their Activity Screen Results as discussed in more detail in Example 100. If there is any discrepancy between a compound's depicted chemical structure and its chemical name, the depicted chemical structure will control.

TABLE 2
Activity Screen Results (see Example 100 below)

| | ADPGlo |
|---|---|
| Compound Number | IC50 (nM) |
| 1 | 672 |
| 2 | 427 |
| 3 | 127 |
| 4 | 545 |
| 5 | 139 |
| 6 | 7667 |
| 7 | 171 |
| 8 | 2782 |
| 9 | 190 |
| 10 | 710 |
| 11 | 2808 |
| 12 | 2587 |
| 13 | 711 |
| 14 | 2196 |
| 15 | 420 |
| 16 | 552 |
| 17 | 526 |
| 18 | 5873 |
| 19 | 3277 |
| 20 | 3957 |
| 21 | 7448 |
| 22 | 358 |
| 23 | 1193 |
| 24 | 995 |
| 25 | 255 |
| 26 | 362 |
| 27 | 414 |
| 28 | 909 |
| 29 | 2303 |
| 30 | 1469 |
| 31 | 1186 |
| 32 | 1443 |
| 33 | 2543 |
| 34 | 387 |
| 35 | 7974 |
| 36 | 1152 |
| 37 | 2505 |
| 38 | 4909 |
| 39 | 3576 |
| 40 | 7526 |
| 41 | 10442 |
| 42 | 10706 |
| 43 | 11407 |
| 44 | 7781 |
| 45 | 5205 |
| 46 | 6036 |
| 47 | 714 |
| 48 | 4682 |
| 49 | 6563 |
| 50 | 6976 |
| 51 | 2779 |
| 52 | 193 |
| 53 | 1592 |
| 54 | 2124 |
| 55 | 2130 |
| 56 | 3277 |
| 57 | 3296 |
| 58 | 4052 |
| 59 | 5580 |
| 60 | 6445 |
| 61 | 7391 |
| 62 | 10505 |
| 63 | 4739 |
| 64 | 3717 |
| 65 | 3791 |
| 66 | 4912 |
| 67 | 5145 |
| 68 | 6337 |
| 69 | 6426 |
| 70 | 8715 |
| 71 | 9966 |
| 72 | 11513 |
| 73 | 11578 |
| 74 | 15814 |
| 75 | 16060 |
| 76 | 16652 |
| 77 | 18918 |
| 78 | 19232 |
| 79 | 22241 |
| 80 | 22866 |
| 81 | 25785 |
| 82 | 26395 |
| 83 | 30975 |
| 84 | 32077 |
| 85 | 34094 |
| 86 | 36079 |
| 87 | 37688 |
| 88 | 38153 |
| 89 | 42658 |
| 90 | 45025 |
| 91 | >66666 |
| 92 | >66666 |
| 93 | >66666 |
| 94 | >66666 |
| 95 | >66666 |
| 96 | >66666 |
| 97 | >66666 |
| 98 | >66666 |
| 99 | >66666 |
| 100 | >66666 |
| 101 | >66666 |
| 102 | >66666 |
| 103 | >66666 |
| 104 | >66666 |
| 105 | 3193 |
| 106 | 4217 |
| 107 | 6445 |

ADPGlo IC50 values are the geometric mean of a minimum of two values.

### GENERAL PROCEDURES

The following abbreviations may be used herein:
ACN: Acetonitrile
aq.: Aqueous
Boc₂O: Di-tert-butyl dicarbonate
Conc.: Concentrated
DCM: Dichloromethane
DCE: 1,2-Dichloroethane
DIAD: Diisopropyl azodicarboxylate
DIPEA: N,N-diisopropylethylamine, Hunig's base
DMAP: 4-(Dimethylamino)pyridine
DMF: Dimethylformamide
DMSO: Dimethylsulfoxide
ESI-MS: Electrospray ionization—mass spectrometry
Et₂O: Diethyl ether
EtOH: Ethanol
EtOAc: Ethyl acetate
Et₃N: Triethylamine
KOH: Potassium hydroxide
LCMS: Liquid chromatography—mass spectrometry
MeOH: Methanol
MW: Microwave
NH₄Cl: Ammonium chloride
NMR: Nuclear magnetic resonance
on or o.n.: overnight
prep-HPLC: Preparative high-performance liquid chromatography
prep-TLC: Preparative thin layer chromatography
r.b.: round-bottom
RT or r.t.: Room temperature
T3P: 1-Propanephosphonic anhydride
TEA: Triethylamine
TFA: Trifluoroacetic acid
THF: Tetrahydrofuran
TLC: Thin-layer chromatography

### General Procedures:

Solvent removal was carried out using either a Buchi rotary evaporator or a Genevac centrifugal evaporator. Preparative LC/MS was conducted using a Waters mass directed autopurification system and a Waters 19×100 mm XBridge 5 micron C18 column under basic mobile phase conditions or an equivalent Waters CSH C18 column under acidic conditions. NMR spectra were recorded using a Bruker Ascend 400 MHz spectrometer. Chemical shifts (δ) are reported in ppm relative to the residual solvent signal (measurement range—6.4 kHz). ¹H NMR data are reported as follows: chemical shift (multiplicity, coupling constants and number of hydrogens). Multiplicity is abbreviated as follows: s (singlet), d (doublet), t (triplet), q (quartet), m (multiplet), br (broad).

When the term “inerted” is used to describe a reactor (e.g., a reaction vessel, flask, glass reactor, and the like) it is meant that the air in the reactor has been replaced with an essentially moisture-free or dry, inert gas (such as nitrogen, argon, and the like).

General methods and experimental procedures for preparing compounds of the present invention are set forth below. In certain cases, a particular compound is described by way of example. However, it will be appreciated that in each case a series of compounds of the present invention were prepared in accordance with the schemes and experimental procedures described below.

### Preparative HPLC Conditions for the Purification of Target Compounds Chromatography Conditions:

Prep HPLC Instrument: Waters 2545 pump with 2767 fraction collector
Column: For mobile phase (2)Waters Xbridge C18 100 mm×19 mm,5 μm particle size
For mobile phase (1)Waters CSH C18 100 mm×19 mm, 5 m particle size
MS Detector: Waters 3100 mass detector
UV detector: Waters 2489 dual wavelength UV detector
Flow Rate: 30 mL/min
Example Gradient Time:

| Time(min) | B % |
|---|---|
| 0 | 20 |
| 1.5 | 20 |
| 6.5 | 40 |
| 6.55 | 95 |
| 8.5 | 95 |

Representative Mobile Phase:
(1)
Mobile Phase: A: 0.1% formic acid in water
Mobile Phase: B: 0.1% formic acid in ACN
(2)
Mobile Phase: A: 0.1% NH₄OH in water
Mobile Phase: B: 0.1% NH₄OH in CAN

Other preparative HPLC conditions for the Purification of Target Compounds

Chromatography conditions:
Prep HPLC Instrument: Shimadzu
Column: Ascentis Express C18 or Shim-Pack XR-ODS C18
Detector: SPD-M20A
Flow Rate: 1.2 mL/min
Representative Mobile Phase:
(1)
Mobile Phase: A: 0.05% formic acid in water
Mobile Phase: B: 0.05% formic acid in ACN

Preparative SFC Conditions for the Purification of Target Compounds

Chromatography Conditions:
SFC Instrument: Thar SFC Prep investigator (Waters)
Column: Chiral Technologies chiralpak IA 250 mm×10 mm,5 m particle size
ELS Detector: Waters 2424 detector
UV detector: Waters 2998 photodiode array detector, 254 nm
Flow Rate: 10 mL/min
Isocratic run: 40% isopropanol as a cosolvent

UPLC, HPLC and MS data provided in the examples described below are registered on:

UPLC Waters Acquity SD
Method name: 1c-ms1-2-ba

Equipment:
UPLC Waters Acquity SQZ
column: Waters Acquity UPLC CSH C18, 50 mm×2.1 mm×1.8 m

Eluents:
(A) 0.1% formic acid in ACN
(B) 0.1% formic acid in water

Analytical method:
Autosampler: Waters2707
injection volume: 1 μL
Pump:

| Time [min] | Flow [ml/min] | % B |
|---|---|---|
| 0.00 | 0.8 | 95 |
| 0.20 | 0.8 | 95 |
| 1.5 | 0.8 | 10 |
| 1.75 | 0.8 | 10 |
| 1.85 | 0.8 | 0 |
| 2.00 | 0.8 | 0 |
| 2.05 | 0.8 | 95 |

Column compartment:

column temperature: ambient

time of analysis: 2.5 min
Detector:

SQZ

ELSD

PDA

### Materials:

Typical starting materials are commercially available and/or can be prepared in a number of ways well known to one skilled in the art of organic synthesis. In the description of the synthetic methods described below, it is to be understood that all proposed reaction conditions, including choice of solvent, reaction atmosphere, reaction temperature, duration of the experiment, and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

Procedure A ((2R,6R)-4-(2-Fluoro-4-(trifluoromethoxy)benzoyl)-2,6-dimethylpiperazin-1-yl)(2-fluoro-4-methoxyphenyl)methanone.

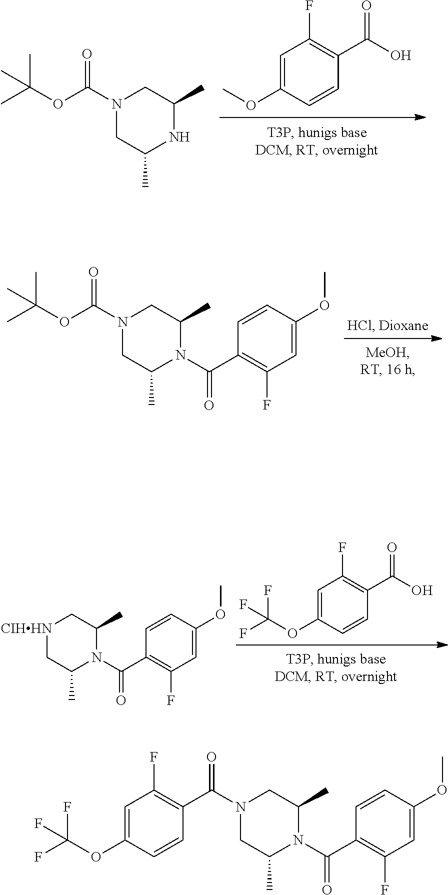

a. tert-Butyl (3R,5R)-4-(2-fluoro-4-methoxybenzoyl)-3,5-dimethylpiperazine-1-carboxylate.

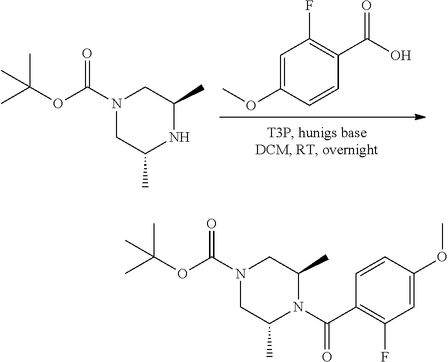

To a r.b flask was added 2-fluoro-4-methoxybenzoic acid 1.19 g, 6.99 mmol) followed by DCM (46.7 mL, 4.67 mmol), hunig'sbase (2.445 mL, 13.998 mmol) and 1-propanephosphonic acid cyclic anhydride [50% in EtOAc (16.3 mmol)]. After stirring for 5 mins at RT, tert-butyl (2R,6R)-2,6-dimethylpiperazine-1-carboxylate (1.0 g, 4.66 mmol) was added and the reaction was stirred overnight at RT. The reaction was quenched with water (20 mL), the organic layer separated and washed with water (20 mL), dried over MgSO₄, filtered and concentrated to give a curde yellow oil. The crude material was purified by flash chromatography on silica eluting with hexane:EtOAc (0-100% gradient) to obtain the title compound (0.90 g, 2.64 mmol, 53% yield) as a white solid. ¹H-NMR (400 MHz, DMSO-d6) δ7.32 (dd, 1H), 6.92 (dd, 1H), 6.85 (dd, 1H), 3.81 (s, 3H), 3.63-3.52 (m, 3H), 3.48-3.32 (m, 3H), 1.42 (s, 9H), 1.19-1.08 (m, 6H). ESI-MS: 367.1 [M+H]⁺.
b. ((2R,6R)-2,6-Dimethylpiperazin-1-yl)(2-fluoro-4-methoxyphenyl)methanone hydrochloride.

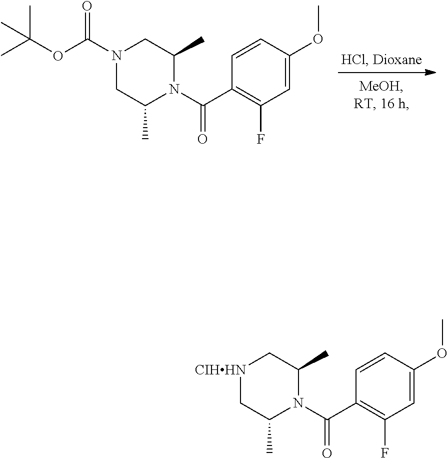

To a r.b flask containing tert-butyl (3R,5R)-4-(2-fluoro-4-methoxybenzoyl)-3,5-dimethylpiperazine-1-carboxylate (903 mg, 2.46 mmol) was added MeOH (4.5 mL) and hydrochloric acid solution [(4.OM in dioxane), 3.1 mL, 12.32 mmol] and the reaction stirred at RT for 2h. After such time, the reaction was concentrated to afford the title compound (745 mg, 2.46 mmol, 100% yield) as a white solid. ¹H-NMR (400 MHz, DMSO-d6) δ 9.73 (br s, 2H), 7.38 (d, 1H), 6.94 (dd, 1H), 6.87 (dd, 1H), 4.11 (br s, 2H), 3.81 (s, 3H), 3.44-3.34 (m, 2H), 3.19-3.13 (m, 2H), 1.28 (s, 3H), 1.26 (s, 3H). ESI-MS: 267.5 [M+H]⁺.

### c. Example 1 ((2R,6R)-4-(2-Fluoro-4-(trifluoromethoxy)benzoyl)-2,6-dimethylpiperazin-1-yl)(2-fluoro-4-methoxyphenyl)methanone (Compound 1)

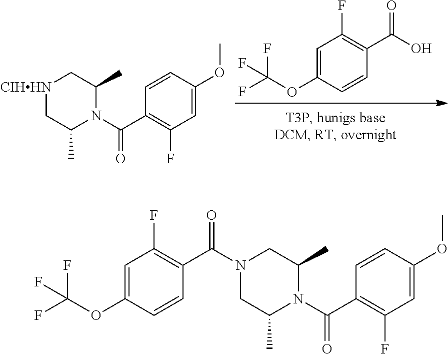

To a reaction vessel was added a solution of ((2R,6R)-2,6-dimethylpiperazin-1-yl)(2-fluoro-4-methoxyphenyl)methanone, HCl (17 mg, 0.056 mmol), DCM (0.56 mL, 0.056 mmol), 1-propanephosphonic acid cyclic anhydride [50% in EtOAc (0.112 mmol)], 2-fluoro-4-(trifluoromethoxy)benzoic acid (0.019 g, 0.085 mmol) and hunig's base (0.029 mL, 0.168 mmol). The reaction was stirred at RT for 16h, after which time the reaction was concentrated and purified by reverse phase HPLC to afford the title compound (15.6 mg, 0.033 mmol, 59%) as a white solid. ¹H-NMR (400 MHz, CDCl₃) δ 7.53-7.49 (dd, 1H), 7.31-7.27 (m, 1H), 7.16-7.14 (m, 1H), 7.05 (dd, 1H), 6.78 (dd, 1H), 6.65 (dd, 1H), 4.48-2.22 (m, 2H), 4.07 (dd, 1H), 3.93 (br d, 1H), 3.85 (s, 3H), 3.79 (dd, 1H), 3.29 (br d, 1H), 1.36 (br s, 3H), 1.25 (br s, 3H). ESI-MS: 473.5 [M+H]⁺.

### Example 2

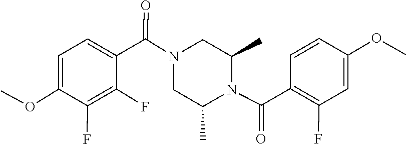

((2R,6R)-4-(2,3-Difluoro-4-methoxybenzoyl)-2,6-dimethylpiperazin-1-yl)(2-fluoro-4-methoxyphenyl)methanone (Compound 2)
The title compound was synthesized following the approach outlined in Procedure A substituting 2,3-difluoro-4-methoxybenzoic acid for 2-fluoro-4-(trifluoromethoxy)benzoic acid in step (c) to afford the title compound (11.6 mg, 0.026 mmol, 47%) as a white solid. ¹H-NMR (400 MHz, CDCl₃) δ 7.31-7.27 (m, 1H), 7.21-7.17 (m, 1H), 6.87-6.63 (m, 1H), 6.78 (dd, 1H), 6.65 (dd, 1H,), 4.39-4.26 (br m, 2H), 4.02 (dd, 1H), 3.98-3.94 (m, 1H), 3.96 (s, 3H), 3.85 (s, 3H), 3.78 (dd, 1H), 3.32 (br d), 1.36 (br s, 3H), 1.23 (br s, 3H). ESI-MS: 437.6 [M+H]⁺.

### Example 3

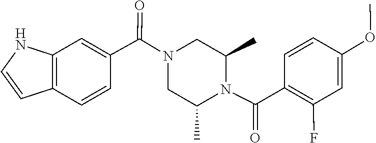 [SMILES: COc1ccc(C(=O)N2[C@H](C)CN(C(=O)c3ccc4cc[nH]c4c3)C[C@H]2C)c(F)c1]

((2R,6R)-4-(1H-Indole-6-carbonyl)-2,6-dimethylpiperazin-1-yl)(2-fluoro-4-methoxyphenyl)methanone (Compound 3)
The title compound was synthesized following the approach outlined in Procedure A substituting 1H-indole-6-carboxylic acid for 2-fluoro-4-(trifluoromethoxy)benzoic acid in step (c) to afford the title compound (242 mg, 0.59 mmol, 89%) as a white solid. ¹H-NMR (400 MHz, CDCl₃) δ 8.67 (s, 1H), 7.58 (d, 1H), 7.53 (s, 1H), 7.23-7.22 (m, 1H), 7.19-7.16 (m, 1H), 7.13-7.09 (m, 1H), 6.68 (dd, 1H), 6.56 (dd, 1H), 6.50 (br s, 1H), 3.96-3.85 (m, 3H), 3.83-3.74 (m, 2H), 3.75 (s, 3H), 3.55-3.43 (m, 1H), 1.33 (br s, 3H), 1.10 (br s, 3H). ESI-MS: 410.2 [M+H]⁺.

### Example 4

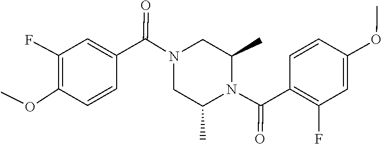

((2R,6R)-4-(3-Fluoro-4-methoxybenzoyl)-2,6-dimethylpiperazin-1-yl)(2-fluoro-4-methoxyphenyl)methanone (Compound 4)
The title compound was synthesized following the approach outlined in Procedure A substituting 3-fluoro-4-methoxybenzoic acid for 2-fluoro-4-(trifluoromethoxy)benzoic acid in step (c) to afford the title compound (12 mg, 0.028 mmol, 51%) as a white solid. ¹H-NMR (400 MHz, DMSO-d6) δ 7.44 (dd, 1H), 7.37-7.31 (m, 2H), 7.25-7.20 (1H, m), 6.93 (dd, 1H), 6.86 (dd, 1H), 4.07-4.01 (m, 1H), 3.89 (s, 3H), 3.81 (s, 3H), 3.77-3.70 (m, 1H), 3.68-3.58 (m, 1H), 3.44-3.35 (m, 1H), 3.34-3.31 (m, 2H), 1.30 (br s, 3H), 1.03 (br s, 3H). ESI-MS: 419.5 [M+H]⁺.

### Example 5

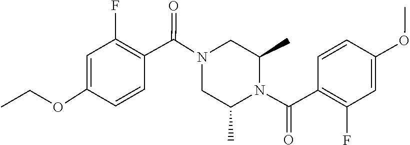 [SMILES: CCOc1ccc(C(=O)N2C[C@@H](C)N(C(=O)c3ccc(OC)cc3F)[C@H](C)C2)c(F)c1]

((2R,6R)-4-(4-Ethoxy-2-fluorobenzoyl)-2,6-dimethylpiperazin-1-yl)(2-fluoro-4-methoxyphenyl)methanone (Compound 5)
The title compound was synthesized following the approach outlined in Procedure A substituting 4-ethoxy-2-fluorobenzoic acid for 2-fluoro-4-(trifluoromethoxy)benzoic acid in step (c) to afford the title compound (14.7 mg, 0.034 mmol, 61%) as a white solid. ¹H-NMR (400 MHz, DMSO-d6) δ 7.38-7.31 (m, 2H), 6.95-6.89 (m, 2H), 6.87-6.84 (m, 2H), 4.09 (q, 2H), 3.86 (d, 1H), 3.80 (s, 3H), 3.82-3.77 (m, 1H), 3.63-6.58 (m, 1H), 3.35-3.31 (m, 1H), 3.24 (d, 1H), 1.34 (t, 3H), 1.23 (br s, 3H), 1.08 (br sm, 3H). ESI-MS: 433.6 [M+H]⁺.

### Example 6

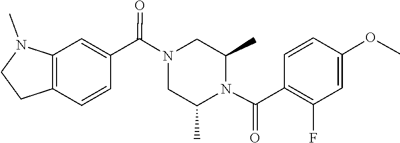 [SMILES: COc1ccc(C(=O)N2[C@H](C)CN(C(=O)c3ccc4c(c3)N(C)CC4)C[C@H]2C)c(F)c1]

((2R,6R)-2,6-Dimethyl-4-(1-methylindoline-6-carbonyl)piperazin-1-yl)(2-fluoro-4-methoxyphenyl)methanone (Compound 6)
The title compound was synthesized following the approach outlined in Procedure A substituting 1-methylindoline-6-carboxylic acid for 2-fluoro-4-(trifluoromethoxy)benzoic acid in step (c) to afford the title compound (23.9 mg, 0.056 mmol, 85%) as a white solid. ¹H-NMR (400 MHz, DMSO-d6) δ 7.33 (dd, 1H), 7.08 (d, 1H), 6.92 (dd, 1H), 6.85 (dd, 1H), 6.67 (dd, 1H,), 6.54 (d, 1H), 3.97-3.90 (m, 2H), 3.80 (s, 3H), 3.79-3.74 (m, 1H), 3.62-3.57 (m, 1H), 3.40-3.32 (m, 2H), 3.29 (t, 2H), 2.90 (t, 2H), 2.73 (s, 3H), 1.28 (br s, 3H), 1.04 (br s, 3H). ESI-MS: 426.7 [M+H]⁺.

### Example 7

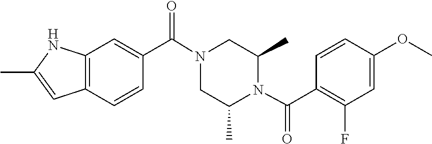

((2R,6R)-2,6-Dimethyl-4-(2-methyl-1H-indole-6-carbonyl)piperazin-1-yl)(2-fluoro-4-methoxyphenyl)methanone (Compound 7)
The title compound was synthesized following the approach outlined in Procedure A substituting 2-methyl-1H-indole-6-carboxylic acid for 2-fluoro-4-(trifluoromethoxy)benzoic acid in step (c) to afford the title compound (13.2 mg, 0.031 mmol, 47%) as a white solid. ESI-MS: 424.4 [M+H]⁺.

### Example 8

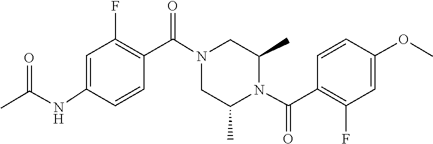 [SMILES: COc1ccc(C(=O)N2[C@H](C)CN(C(=O)c3ccc(NC(C)=O)cc3F)C[C@H]2C)c(F)c1]

N-(3-Fluoro-4-((3R,5R)-4-(2-fluoro-4-methoxybenzoyl)-3,5-dimethylpiperazine-1-carbonyl)phenyl)acetamide (Compound 8)
The title compound was synthesized following the approach outlined in Procedure A substituting 4-acetamido-2-fluorobenzoic acid for 2-fluoro-4-(trifluoromethoxy)benzoic acid in step (c) to afford the title compound (17.0 mg, 0.038 mmol, 68%) as a white solid. ¹H-NMR (400 MHz, CDCl₃) δ 7.81 (s, 1H), 7.62 (d, 1H), 7.35 (t, 1H), 7.29-7.26 (m, 1H), 7.13 (dd, 1H), 6.77 (dd, 1H), 6.65 (dd, 1H), 4.45-2.15 (br s, 2H), 4.04 (dd, 1H), 3.94 (br dd, 1H), 3.84 (s, 3H), 3.78 (dd, 1H), 3.32 (d, 1H), 2.20 (s, 3H), 1.36 (br s, 3H), 1.22 (br s, 3H). ESI-MS: 446.4 [M+H]⁺.

### Example 9

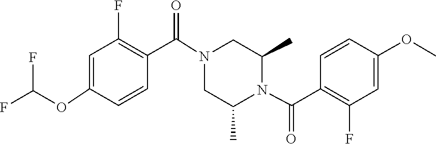 [SMILES: COc1ccc(C(=O)N2[C@H](C)CN(C(=O)c3ccc(OC(F)F)cc3F)C[C@H]2C)c(F)c1]

((2R,6R)-4-(4-(Difluoromethoxy)-2-fluorobenzoyl)-2,6-dimethylpiperazin-1-yl)(2-fluoro-4-methoxyphenyl)methanone (Compound 9)
The title compound was synthesized following the approach outlined in Procedure A substituting 4-(difluoromethoxy)-2-fluorobenzoic acid for 2-fluoro-4-(trifluoromethoxy)benzoic acid in step (c) to afford the title compound (13.1 mg, 0.028 mmol, 51%) as a white solid. ¹H-NMR (400 MHz, CDCl₃) δ 7.48 (dd, 1H), 7.31-7.27 (m, 1H), 7.04 (dd, 1H), 6.95 (dd, 1H), 6.79-6.76 (m, 1H), 6.65 (dd, 1H), 6.49 (d, 1H), 4.50-4.20 (br m, 2H), 4.06 (dd, 1H), 3.93 (br dd, 1H), 3.84 (s, 3H), 3.78 (dd, 1H), 3.30 (br d, 1H), 1.36 (br s, 3H), 1.24 (br s, 3H). ESI-MS: 455.6 [M+H]⁺.

### Example 10

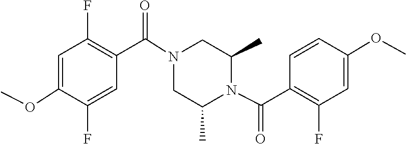

((2R,6R)-4-(2,5-Difluoro-4-methoxybenzoyl)-2,6-dimethylpiperazin-1-yl)(2-fluoro-4-methoxyphenyl)methanone (Compound 10)
The title compound was synthesized following the approach outlined in Procedure A substituting 2,5-difluoro-4-methoxybenzoic acid for 2-fluoro-4-(trifluoromethoxy)benzoic acid in step (c) to afford the title compound (12.6 mg, 0.028 mmol, 51%) as a white solid. ¹H-NMR (400 MHz, CDCl₃) δ 7.31-7.27 (m, 1H), 7.21 (dd, 1H), 6.79-6.76 (m, 1H), 6.73 (dd, 1H), 6.65 (dd, 1H), 4.46-4.21 (m, 2H), 4.03-3.99 (m, 1H), 3.98-3.92 (m, 1H), 3.93 (s, 3H), 3.85 (s, 3H), 3.77 (dd, 1H), 3.34 (br d, 1H), 1.35 (br s, 3H), 1.23 (br s, 3H). ESI-MS: 437.5 [M+H]⁺.

### Example 11

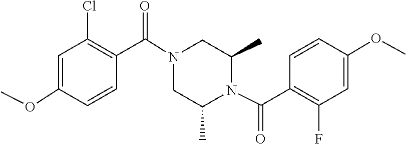

((2R,6R)-4-(2-Chloro-4-methoxybenzoyl)-2,6-dimethylpiperazin-1-yl)(2-fluoro-4-methoxyphenyl)methanone (Compound 11)
The title compound was synthesized following the approach outlined in Procedure A substituting 2-chloro-4-methoxybenzoic acid for 2-fluoro-4-(trifluoromethoxy)benzoic acid in step (c) to afford the title compound (16.6 mg, 0.038 mmol, 68%) as a white solid. ESI-MS: 435.4 [M+H]⁺.

### Example 12

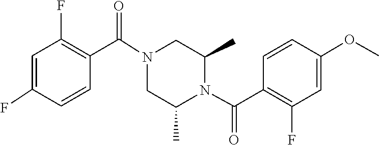 [SMILES: COc1ccc(C(=O)N2[C@H](C)CN(C(=O)c3ccc(F)cc3F)C[C@H]2C)c(F)c1]

((2R,6R)-4-(2,4-Difluorobenzoyl)-2,6-dimethylpiperazin-1-yl)(2-fluoro-4-methoxyphenyl)methanone (Compound 12)
The title compound was synthesized following the approach outlined in Procedure A substituting 2,4-difluorobenzoic acid for 2-fluoro-4-(trifluoromethoxy)benzoic acid in step (c) to afford the title compound (12.9 mg, 0.032 mmol, 56%) as a white solid. ¹H-NMR (400 MHz, CDCl₃) δ 7.50-7.44 (m, 1H), 7.31-7.27 (m, 1H), 7.03-6.98 (m, 1H), 6.90 (ddd, 1H), 6.78 (dd, 1H), 6.65 (dd, 1H), 4.55-4.20 (br s, 2H), 4.05 (dd, 1H), 3.93 (br dd, 1H), 3.84 (s, 3H), 3.78 (dd, 1H), 3.29 (br d, 1H), 1.36 (br s, 3H), 1.24 (br s, 3H). ESI-MS: 407.4 [M+H]⁺.

### Example 13

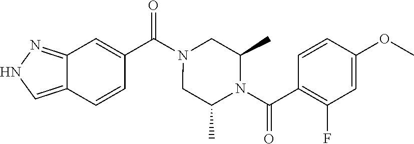 [SMILES: COc1ccc(C(=O)N2[C@H](C)CN(C(=O)c3ccc4c[nH]nc4c3)C[C@H]2C)c(F)c1]

((2R,6R)-4-(2H-Indazole-6-carbonyl)-2,6-dimethylpiperazin-1-yl)(2-fluoro-4-methoxyphenyl)methanone (Compound 13)
The title compound was synthesized following the approach outlined in Procedure A substituting 6-indazolecarboxylic acid for 2-fluoro-4-(trifluoromethoxy)benzoic acid in step (c) to afford the title compound (20.1 mg, 0.051 mmol, 78%) as a white solid. ESI-MS: 411.7 [M+H]⁺.

### Example 14

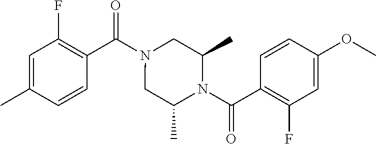

((3R,5R)-4-(2-Fluoro-4-methoxybenzoyl)-3,5-dimethylpiperazin-1-yl)(2-fluoro-4-methylphenyl)methanone (Compound 14)
The title compound was synthesized following the approach outlined in Procedure A substituting 2-fluoro-4-methylbenzoic acid for 2-fluoro-4-(trifluoromethoxy)benzoic acid in step (c) to afford the title compound (12.4 mg, 0.031 mmol, 55%) as a white solid. ¹H-NMR (400 MHz, CDCl₃) δ 7.35-7.31 (m, 1H), 7.30-7.26 (m, 1H), 7.05 (d, 1H), 6.95 (d, 1H), 6.77 (dd, 1H), 6.65 (dd, 1H), 4.50-4.15 (br s, 2H), 4.06 (dd, 1H), 3.91 (br d, 1H), 3.84 (s, 3H), 3.79-3.75 (m, 1H), 3.31 (br d, 1H), 2.41 (s, 3H), 1.35 (br s, 3H), 1.23 (br s, 3H). ESI-MS: 403.6 [M+H]⁺.

### Example 15

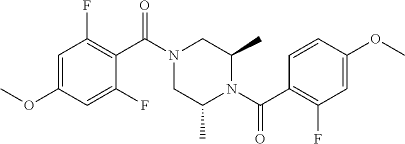

((2R,6R)-4-(2,6-Difluoro-4-methoxybenzoyl)-2,6-dimethylpiperazin-1-yl)(2-fluoro-4-methoxyphenyl)methanone (Compound 15)
The title compound was synthesized following the approach outlined in Procedure A substituting 2,6-difluoro-4-methoxybenzoic acid for 2-fluoro-4-(trifluoromethoxy)benzoic acid in step (c) to afford the title compound (13.3 mg, 0.030 mmol, 54%) as a white solid. 1H-NMR (400 MHz, CDCl₃) δ 7.31-7.27 (m, 1H), 6.78 (dd, 1H), 6.65 (dd, 1H), 6.56-6.50 (m, 2H), 4.50-4.20 (br s, 2H), 4.12 (dd, 1H), 3.88-3.82 (m, 1H), 3.85 (s, 3H), 3.84 (s, 3H), 3.76 (dd, 1H), 3.33 (dd, 1H), 1.33 (br s, 3H), 1.28 (br s, 3H). ESI-MS: 437.6 [M+H]⁺.

### Example 16

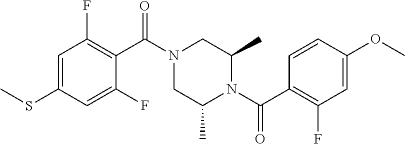

((2R,6R)-4-(2,6-Difluoro-4-(methylthio)benzoyl)-2,6-dimethylpiperazin-1-yl)(2-fluoro-4-methoxyphenyl)methanone (Compound 16)
The title compound was synthesized following the approach outlined in Procedure A substituting 2,6-difluoro-4-(methylthio)benzoic acid for 2-fluoro-4-(trifluoromethoxy)benzoic acid in step (c) to afford the title compound (19.9 mg, 0.044 mmol, 66%) as a white solid. ¹H-NMR (400 MHz, CDCl₃) δ 7.31-7.27 (m, 1H), 6.85-6.81 (m, 2H), 6.78 (dd, 1H), 6.65 (dd, 1H), 4.45-1.21 (br m, 1H), 4.12 (dd, 1H), 3.87-3.82 (m, 1H), 3.84 (s, 3H), 3.78-3.73 (m, 2H), 3.32 (dd, 1H), 2.52 (s, 3H), 1.35 (br s, 3H), 1.28 (br s, 3H). ESI-MS: 453.6 [M+H]⁺.

### Example 17

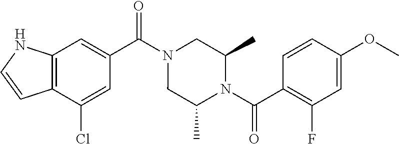 [SMILES: COc1ccc(C(=O)N2[C@H](C)CN(C(=O)c3cc(Cl)c4cc[nH]c4c3)C[C@H]2C)c(F)c1]

(4-Chloro-1H-indol-6-yl)((3R,5R)-4-(2-fluoro-4-methoxybenzoyl)-3,5-dimethylpiperazin-1-yl)methanone (Compound 17)
The title compound was synthesized following the approach outlined in Procedure A substituting 4-chloro-1H-indole-6-carboxylic acid for 2-fluoro-4-(trifluoromethoxy)benzoic acid in step (c) to afford the title compound (13.3 mg, 0.029 mmol, 45%) as a white solid. ESI-MS: 444.5 [M+H]⁺.

### Example 18

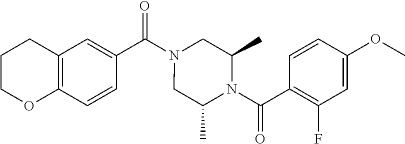 [SMILES: COc1ccc(C(=O)N2[C@H](C)CN(C(=O)c3ccc4c(c3)CCCO4)C[C@H]2C)c(F)c1]

Chroman-6-yl((3R,5R)-4-(2-fluoro-4-methoxybenzoyl)-3,5-dimethylpiperazin-1-yl)methanone (Compound 18)

The title compound was synthesized following the approach outlined in Procedure A substituting chromane-6-carboxylic acid for 2-fluoro-4-(trifluoromethoxy)benzoic acid in step (c) to afford the title compound (19.4 mg, 0.045 mmol, 69%) as a white solid. 1H-NMR (400 MHz, CDCl₃) δ 7.20-7.26 (m, 1H), 7.24 (d, 1H), 7.22-7.20 (m, 1H), 6.81 (d, 1H), 6.77 (dd, 1H), 6.65 (dd, 1H), 4.42-4.20 (br s, 1H), 4.25-4.22 (m, 2H), 4.05-3.85 (m, 3H), 3.84 (s, 3H), 3.86-3.76 (m, 1H), 3.62-3.63 (m, 1H), 2.85-2.80 (m, 2H), 2.07-2.00 (m, 2H), 1.38 (br s, 3H), 1.20 (br s, 3H). ESI-MS: 427.6 [M+H]⁺.

### Example 19

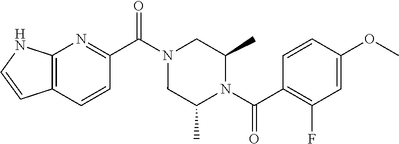 [SMILES: COc1ccc(C(=O)N2[C@H](C)CN(C(=O)c3ccc4cc[nH]c4n3)C[C@H]2C)c(F)c1]

((2R,6R)-2,6-Dimethyl-4-(1H-pyrrolo[2,3-b]pyridine-6-carbonyl)piperazin-1-yl)(2-fluoro-4-methoxyphenyl)methanone (Compound 19)
The title compound was synthesized following the approach outlined in Procedure A substituting 1H-pyrrolo[2,3-b]pyridine-6-carboxylic acid for 2-fluoro-4-(trifluoromethoxy)benzoic acid in step (c) to afford the title compound (13.7 mg, 0.033 mmol, 51%) as a white solid. ¹H-NMR (400 MHz, CDCl₃) δ 9.36 (br s, 1H), 8.05 (d, 1H), 7.56 (d, 1H), 7.47 (dd, 1H), 7.33-7.29 (m, 1H), 6.77 (dd, 1H), 6.68-6.62 (m, 1H), 6.58 (dd, 1H), 4.22 (br dd, 1H), 4.10-4.03 (m, 1H), 3.92-3.85 (m, 1H), 3.84 (s, 3H), 3.81-3.74 (m, 3H), 1.43 (br s, 3H), 1.23 (br s, 3H). ESI-MS: 411.7 [M+H]⁺.

### Example 20

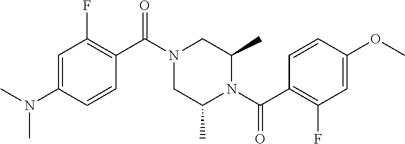

((2R,6R)-4-(4-(Dimethylamino)-2-fluorobenzoyl)-2,6-dimethylpiperazin-1-yl)(2-fluoro-4-methoxyphenyl)methanone (Compound 20)
The title compound was synthesized following the approach outlined in Procedure A substituting 4-(dimethylamino)-2-fluorobenzoic acid for 2-fluoro-4-(trifluoromethoxy)benzoic acid in step (c) to afford the title compound (10.2 mg, 0.023 mmol, 42%) as a white solid. ¹H-NMR (400 MHz, CDCl₃) δ 7.35 (dd, 1H), 7.30-7.26 (m, 1H), 6.77 (dd, 1H), 6.65 (dd, 1H), 6.51 (dd, 1H), 6.33 (dd, 1H), 4.45-4.15 (br m, 2H), 3.98 (br d, 2H), 3.84 (s, 3H), 3.78 (br dd), 3.39 (br d, 1H), 3.04-3.01 (m, 6H), 1.36 (br s, 3H), 1.21 (br s, 3H). ESI-MS: 432.6 [M+H]⁺.

### Example 21

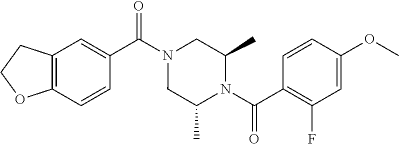 [SMILES: COc1ccc(C(=O)N2[C@H](C)CN(C(=O)c3ccc4c(c3)CCO4)C[C@H]2C)c(F)c1]

((2R,6R)-4-(2,3-Dihydrobenzofuran-5-carbonyl)-2,6-dimethylpiperazin-1-yl)(2-fluoro-4-methoxyphenyl)methanone (Compound 21)
The title compound was synthesized following the approach outlined in Procedure A substituting 2,3-dihydrobenzofuran-5-carboxylic acid for 2-fluoro-4-(trifluoromethoxy)benzoic acid in step (c) to afford the title compound (10.4 mg, 0.025 mmol, 45%) as a white solid. ¹H-NMR (400 MHz, CDCl₃) δ 7.39 (d, 1H), 7.31-7.27 (m, 1H), 7.27-7.25 (m, 1H), 6.81 (d, 1H), 6.77 (dd, 1H), 6.65 (dd, 1H), 4.65 (t, 2H), 4.44-4.20 (br m, 1H), 4.02-3.94 (m, 1H), 3.93-3.78 (m, 3H), 3.84 (s, 3H), 3.61-3.52 (m, 1H), 3.29-3.22 (m, 2H), 1.39 (br s, 3H), 1.20 (br s, 3H). ESI-MS: 413.6 [M+H]⁺.
Procedure B ((2R,6R)-4-(2-Fluoro-4-methoxybenzoyl)-2,6-dimethylpiperazin-1-yl)(2-fluoro-4-methylphenyl)methanone (Compound 22)

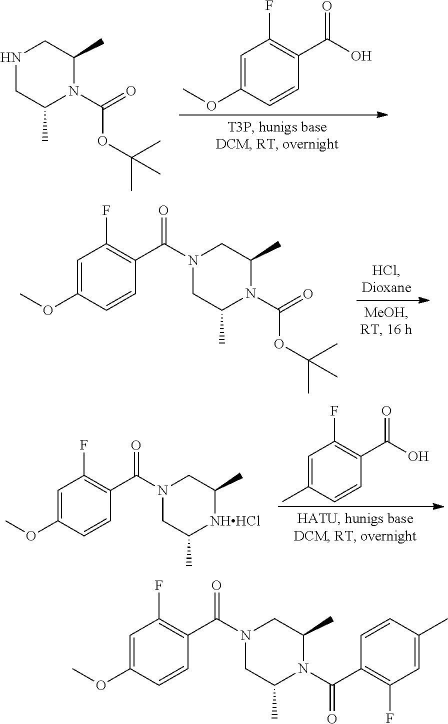

a. tert-Butyl (2R,6R)-4-(2-fluoro-4-methoxybenzoyl)-2,6-dimethylpiperazine-1-carboxylate.

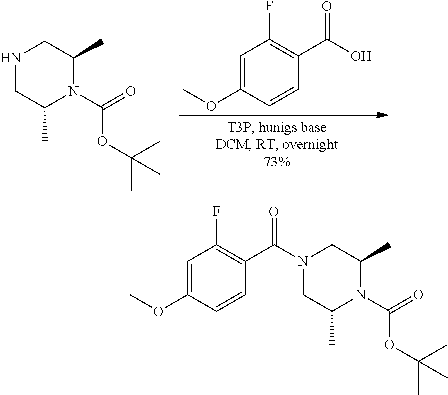 [SMILES: C[C@@H]1CNC[C@@H](C)N1C(=O)OC(C)(C)C ; COc1ccc(C(=O)O)c(F)c1 ; COc1ccc(C(=O)N2C[C@@H](C)N(C(=O)OC(C)(C)C)[C@H](C)C2)c(F)c1]

To a r.b flask was added 2-fluoro-4-methoxybenzoic acid (1.19 g, 6.99 mmol) followed by DCM (46.7 mL, 4.67 mmol), hunig's base (2.45 mL, 13.99 mmol) and 1-propanephosphonic acid cyclic anhydride [50% in EtOAc (9.7 mL, 16.3 mmol)]. After stirring for 5 mins at RT, tert-butyl (2R,6R)-2,6-dimethylpiperazine-1-carboxylate (1.0 g, 4.66 mmol) was added and the reaction was stirred overnight at RT. After such time the reaction was quenched with water (20 mL), the organic layer separated and washed with water (20 mL), dried over MgSO₄, filtered and concentrated to give a crude yellow oil. The crude material was purified by flash chromatography on silica eluting with hexane:EtOAc (0-100% gradient) to afford the title compound (1.25 g, 3.40 mmol, 72.9% yield) as a white solid. ¹H-NMR (400 MHz, DMSO-d6) δ 7.35 (dd, 1H), 6.93 (dd, 1H), 6.87 (dd, 1H), 4.15-4.12 (m, 1H), 3.95-3.92 (m, 1H), 3.82 (s, 3H), 3.81-3.78 (m, 1H), 3.72 (dd, 1H), 3.54 (dd, 1H), 3.18 (dd, 1H), 1.42 (s, 9H), 1.25 (d, 3H), 1.08 (d, 3H). ESI-MS: 367.1 [M+H]⁺.
b. ((3R,5R)-3,5-Dimethylpiperazin-1-yl)(2-fluoro-4-methoxyphenyl)methanone hydrochloride.

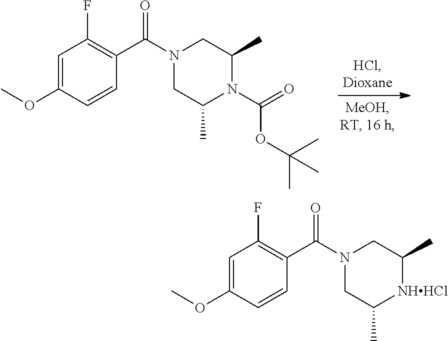

To a r.b flask containing tert-butyl (2R,6R)-4-(2-fluoro-4-methoxybenzoyl)-2,6-dimethylpiperazine-1-carboxylate (1.35 g, 3.67 mmol) was added MeOH (6.7 mL), hydrochloric acid solution [(4.OM in dioxane), 4.6 mL, 18.37 mmol] and the reaction stirred at RT for 2h. After such time, the reaction was concentrated to afford the title compound (1.21 g, 4.00 mmol, 109% yield) as a white solid. ¹H-NMR (400 MHz, DMSO) δ 9.52 (br s, 1H), 7.38 (dd, 1H), 6.95 (dd, 1H), 6.88 (dd, 1H), 3.93 (br s, 1H), 3.82 (s, 3H), 3.65-3.58 (m, 1H), 3.50-3.45 (m, 1H), 3.34-3.32 (m, 2H), 3.28-3.22 (m, 1H), 1.30 (br s, 3H), 1.16 (br s, 3H). ESI-MS: 267.5 [M+H]⁺.

### c. Example 22 ((2R,6R)-4-(2-Fluoro-4-methoxybenzoyl)-2,6-dimethylpiperazin-1-yl)(2-fluoro-4-methylphenyl)methanone (Compound 22)

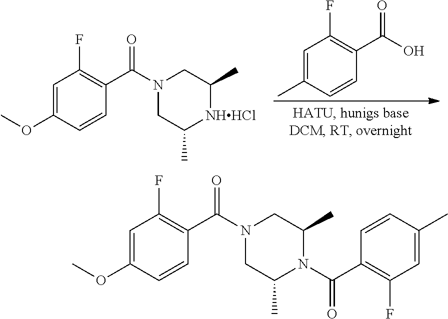

To a reaction vessel was added a solution of ((3R,5R)-3,5-dimethylpiperazin-1-yl)(2-fluoro-4-methoxyphenyl)methanone, HCl (20 mg, 0.066 mmol) in DCM (0.66 mL, 0.066 mmol) followed by HATU (0.132 mmol), 2-fluoro-4-methylbenzoic acid (0.020 g, 0.130 mmol) and hunig'sbase (0.029 ml, 0.165 mmol). The reaction was stirred at RT for 16h, after which time the reaction was concentrated and purified by reverse phase HPLC to afford the title compound (8.5 mg, 0.021 mmol, 32%) as a white solid. ¹H-NMR (400 MHz, DMSO-d6) δ 7.37 (dd, 1H), 7.30 (dd, 1H), 7.15-7.09 (m, 2H), 6.94 (dd, 1H), 6.87 (dd, 1H), 4.60-4.3 (br m, 1H), 3.89-3.80 (m, 1H), 3.81 (s, 3H), 3.80-3.77 (m, 1H), 3.63-3.57 (m, 1H), 3.34-3.29 (m, 1H), 3.28-3.22 (m, 1H), 2.35 (s, 3H), 1.35-1.11 (br s, 6H). ESI-MS: 403.7 [M+H]⁺.

### Example 23

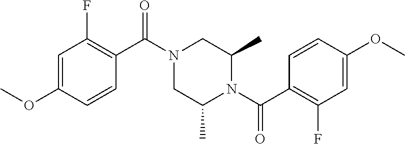

((3R,5R)-4-(2,5-Difluoro-4-methoxybenzoyl)-3,5-dimethylpiperazin-1-yl)(2-fluoro-4-methoxyphenyl)methanone (Compound 23) The title compound was synthesized following the approach outlined in Procedure B substituting 2,5-difluoro-4-methoxybenzoic acid for 2-fluoro-4-methylbenzoic acid in step (c) to afford the title compound (1.1 mg, 0.002 mmol, 4%) as a white solid. ESI-MS: 437.4 [M+H]⁺.

### Example 24

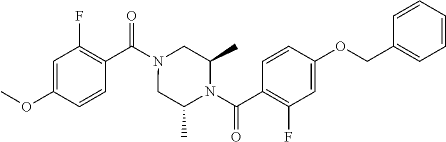 [SMILES: COc1ccc(C(=O)N2C[C@@H](C)N(C(=O)c3ccc(OCc4ccccc4)cc3F)[C@H](C)C2)c(F)c1]

((3R,5R)-4-(4-(Benzyloxy)-2-fluorobenzoyl)-3,5-dimethylpiperazin-1-yl)(2-fluoro-4-methoxyphenyl)methanone (Compound 24)
The title compound was synthesized following the approach outlined in Procedure B substituting 4-(benzyloxy)-2-fluorobenzoic acid for 2-fluoro-4-methylbenzoic acid in step (c) to afford the title compound (10.3 mg, 0.021 mmol, 32%) as a white solid. ¹H-NMR (400 MHz, CDCl₃) δ 7.45-7.42 (m, 4H), 7.41 (m, 2H), 7.32-7.26 (m, 1H), 6.85 (dd, 1H), 6.79 (dd, 1H), 6.73 (dd, 1H), 6.66 (dd, 1H), 5.09 (s, 2H), 4.54-4.13 (br m, 2H), 4.03 (dd, 1H), 3.95 (br dd, 1H), 3.86 (s, 3H), 3.81-3.75 (m, 1H), 3.33 (br d, 1H), 1.36 (br s, 3H), 1.22 (br s, 3H). ESI-MS: 495.6 [M+H]⁺.

### Example 25

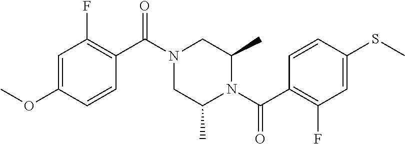

((3R,5R)-4-(2,6-difluoro-4-(methylthio)benzoyl)-3,5-dimethylpiperazin-1-yl)(2-fluoro-4-methoxyphenyl)methanone (Compound 25)
The title compound was synthesized following the approach outlined in Procedure B substituting 2,6-difluoro-4-(methylthio)benzoic acid for 2-fluoro-4-methylbenzoic acid in step (c) to afford the title compound (8.9 mg, 0.019 mmol, 30%) as a white solid. ESI-MS: 453.5 [M+H]⁺.

### Example 26

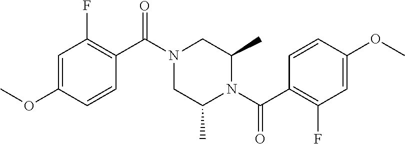

((2R,6R)-2,6-Dimethylpiperazine-1,4-diyl)bis((2-fluoro-4-methoxyphenyl)methanone) (Compound 26)
The title compound was synthesized following the approach outlined in Procedure B substituting 2-fluoro-4-methoxybenzoic acid for 2-fluoro-4-methylbenzoic acid in step (c) to afford the title compound (8.1 mg, 0.019 mmol, 29%) as a white solid. ¹H-NMR (400 MHz, CDCl₃) δ 7.40 (dd, 1H), 7.30-7.26 (m, 1H), 6.78 (ddd, 2H), 6.65 (dd, 2H), 4.50-4.15 (br m, 2H), 4.03 (dd, 1H), 3.95 (br dd, 1H), 3.85 (s, 3H), 3.84 (s, 3H), 3.77 (br dd, 1H), 3.33 (br d), 1.39-1.32 (br m, 3H), 1.25-1.18 (br m, 3H). ESI-MS: 419.3 [M+H]⁺.

### Example 27

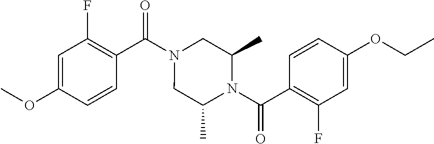 [SMILES: CCOc1ccc(C(=O)N2[C@H](C)CN(C(=O)c3ccc(OC)cc3F)C[C@H]2C)c(F)c1]

((3R,5R)-4-(4-Ethoxy-2-fluorobenzoyl)-3,5-dimethylpiperazin-1-yl)(2-fluoro-4-methoxyphenyl)methanone (Compound 27)
The title compound was synthesized following the approach outlined in Procedure B substituting 4-ethoxy-2-fluorobenzoic acid for 2-fluoro-4-methylbenzoic acid in step (c) to afford the title compound (0.8 mg, 0.002 mmol, 2%) as a white solid. ESI-MS: 433.6 [M+H]⁺.

### Example 28

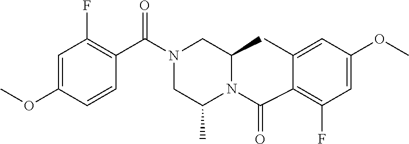

((3R,5R)-4-(2-Fluoro-4-methoxy-6-methylbenzoyl)-3,5-dimethylpiperazin-1-yl)(2-fluoro-4-methoxyphenyl)methanone (Compound 28)
The title compound was synthesized following the approach outlined in Procedure B substituting 2-fluoro-4-methoxy-6-methylbenzoic acid for 2-fluoro-4-methylbenzoic acid in step (c) to afford the title compound (1.3 mg, 0.003 mmol, 5%) as a white solid. ESI-MS: 433.7 [M+H]⁺.

### Example 29

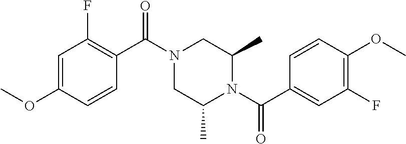

((2R,6R)-4-(2-Fluoro-4-methoxybenzoyl)-2,6-dimethylpiperazin-1-yl)(3-fluoro-4-methoxyphenyl)methanone (Compound 29)
The title compound was synthesized following the approach outlined in Procedure B substituting 3-fluoro-4-methoxybenzoic acid for 2-fluoro-4-methylbenzoic acid in step (c) to afford the title compound (5.3 mg, 0.012 mmol, 19%) as a white solid. ESI-MS: 419.6 [M+H]⁺.

### Example 30

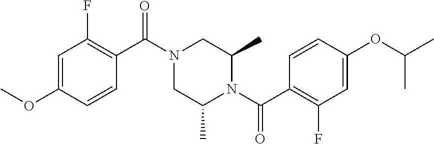 [SMILES: COc1ccc(C(=O)N2C[C@@H](C)N(C(=O)c3ccc(OC(C)C)cc3F)[C@H](C)C2)c(F)c1]

((3R,5R)-4-(2-fluoro-4-isopropoxybenzoyl)-3,5-dimethylpiperazin-1-yl)(2-fluoro-4-methoxyphenyl)methanone (Compound 30)
The title compound was synthesized following the approach outlined in Procedure B substituting 2-fluoro-4-isopropoxybenzoic acid for 2-fluoro-4-methylbenzoic acid to afford the title compound (6.5 mg, 0.014 mmol, 22%) as a white solid. ESI-MS: 447.3 [M+H]⁺.

### Procedure C:

### Example 31

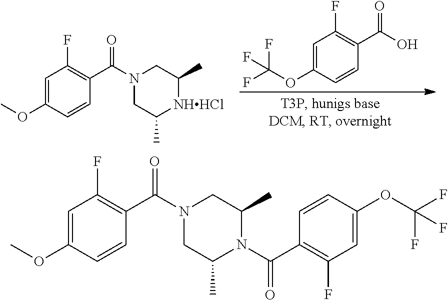

((3R,5R)-4-(2-fluoro-4-(trifluoromethoxy)benzoyl)-3,5-dimethylpiperazin-1-yl)(2-fluoro-4-methoxyphenyl)methanone (Compound 31)
To a 1 dram vial was added a solution of ((3R,5R)-3,5-dimethylpiperazin-1-yl)(2-fluoro-4-methoxyphenyl)methanone, HCl (10 mg, 0.033 mmol), DCM (0.330 mL, 0.033 mmol), 1-Propanephosphonic acid cyclic anhydride [50% in EtOAc (0.066 mmol)]followed by 2-fluoro-4-(trifluoromethoxy)benzoic acid (11 mg, 0.049 mmol) and hunig'sbase (17 μL, 0.099 mmol). The reaction was stirred at RT for 16h, after which time the reaction was concentrated and purified by reverse phase HPLC to afford the title compound (0.4 mg, 8.4×10⁻⁷ mol, 3%) as a white solid. ESI-MS: 473.6 [M+H]⁺.

### Example 32

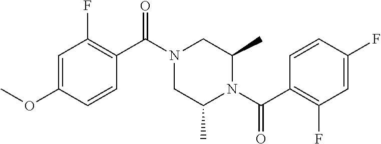 [SMILES: COc1ccc(C(=O)N2C[C@@H](C)N(C(=O)c3ccc(F)cc3F)[C@H](C)C2)c(F)c1]

((3R,5R)-4-(2,4-difluorobenzoyl)-3,5-dimethylpiperazin-1-yl)(2-fluoro-4-methoxyphenyl)methanone (Compound 32)
The title compound was synthesized following the approach outlined in Procedure C substituting 2,4-difluorobenzoic acid for 2-fluoro-4-(trifluoromethoxy)benzoic acid to afford the title compound (2.1 mg, 0.005 mmol, 16%) as a white solid. ESI-MS: 407.6 [M+H]⁺.

### Example 33

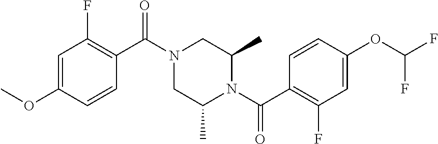 [SMILES: COc1ccc(C(=O)N2C[C@@H](C)N(C(=O)c3ccc(OC(F)F)cc3F)[C@H](C)C2)c(F)c1]

((3R,5R)-4-(4-(difluoromethoxy)-2-fluorobenzoyl)-3,5-dimethylpiperazin-1-yl)(2-fluoro-4-methoxyphenyl)methanone (Compound 33)
The title compound was synthesized following the approach outlined in Procedure C substituting 4-(difluoromethoxy)-2-fluorobenzoic acid for 2-fluoro-4-(trifluoromethoxy)benzoic acid to afford the title compound (5.3 mg, 0.011 mmol, 35%) as a white solid. ESI-MS: 455.3 [M+H]⁺.

### Example 34

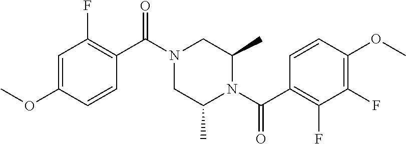

((3R,5R)-4-(2,3-Difluoro-4-methoxybenzoyl)-3,5-dimethylpiperazin-1-yl)(2-fluoro-4-methoxyphenyl)methanone (Compound 34)
The title compound was synthesized following the approach outlined in Procedure C substituting 2,3-difluoro-4-methoxybenzoic acid for 2-fluoro-4-(trifluoromethoxy)benzoic acid to afford the title compound (1.7 mg, 0.004 mmol, 12%) as a white solid. ESI-MS: 437.7 [M+H]⁺.
Procedure D: Example 35, 6-((3R,5R)-4-(2-Fluoro-4-methoxybenzoyl)-3,5-dimethylpiperazine-1-carbonyl)-2H-benzo[b][1,4]oxazin-3(4H)-one (Compound 35)

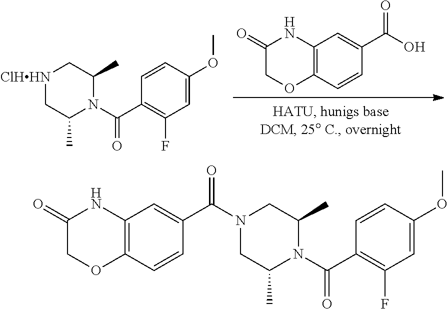

Into a 1 dram vial was added ((2R,6R)-2,6-dimethylpiperazin-1-yl)(2-fluoro-4-methoxyphenyl)methanone hydrochloride (20 mg, 0.066 mmol) in DCM (2.0 mL) followed by 3-oxo-3,4-dihydro-2H-benzo[b][1,4]oxazine-6-carboxylic acid (0.09 mmol), HATU (0.09 mmol) and DIEA (0.225 mmol). The mixture was stirred at 25° C. overnight, after which time the crude mixture was filtered, concentrated and purified by Prep-HPLC directly to afford the title compound (19 mg, 0.043 mmol, 65%) as a white solid. ¹H-NMR (400 MHz, CDCl₃) δ 8.63 (br s, 1H), 7.22-7.18 (m, 1H), 7.03 (d, 1H), 7.02-6.99 (m, 1H), 6.92-6.90 (m, 1H), 6.639 (dd, 1H), 6.56 (dd, 1H), 4.57 (s, 2H), 4.34-4.12 (br m, 2H), 3.94-3.81 (m, 2H), 3.78-3.70 (m, 1H), 3.75 (s, 3H), 3.44 (br d, 1H), 1.29 (br s, 3H), 1.12 (br s, 3H). ESI-MS: 442.4 [M+H]⁺.

### Example 36

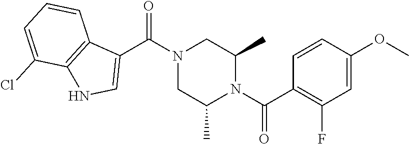 [SMILES: COc1ccc(C(=O)N2[C@H](C)CN(C(=O)c3c[nH]c4c(Cl)cccc34)C[C@H]2C)c(F)c1]

(7-Chloro-1H-indol-3-yl)((3R,5R)-4-(2-fluoro-4-methoxybenzoyl)-3,5-dimethylpiperazin-1-yl)methanone (Compound 36)
The title compound was synthesized following the approach outlined in Procedure D substituting 7-chloro-1H-indole-3-carboxylic acid for 3-oxo-3,4-dihydro-2H-benzo[b][1,4]oxazine-6-carboxylic acid to afford the title compound (17.5 mg, 0.039 mmol, 59%) as a white solid. ¹H-NMR (400 MHz, CDCl₃) δ 8.81 (s, 1H), 7.68 (d, 1H), 7.49 (d, 1H), 7.23-7.17 (m, 2H), 7.12-7.08 (m, 1H), 6.69 (dd, 1H,), 6.56 (dd, 1H), 4.0-3.8 (m, 4H), 3.78-3.6 (m, 2H), 3.76 (s, 3H), 1.33-1.10 (m, 6H). ESI-MS: 444.5 [M+H]⁺.

### Example 37

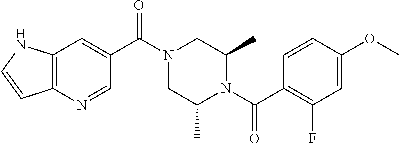 [SMILES: COc1ccc(C(=O)N2[C@H](C)CN(C(=O)c3cnc4cc[nH]c4c3)C[C@H]2C)c(F)c1]

((2R,6R)-2,6-Dimethyl-4-(1H-pyrrolo[3,2-b]pyridine-6-carbonyl)piperazin-1-yl)(2-fluoro-4-methoxyphenyl)methanone (Compound 37)
The title compound was synthesized following the approach outlined in Procedure D substituting 1H-pyrrolo[3,2-b]pyridine-6-carboxylic acid for 3-oxo-3,4-dihydro-2H-benzo[b][1,4]oxazine-6-carboxylic acid to afford the title compound (12.1 mg, 0.029 mmol, 45%) as a white solid. ¹H-NMR (400 MHz, CDCl₃) δ 9.09 (s, 1H), 8.61 (d, 1H), 7.91 (d, 1H), 7.58 (dd, 1H), 7.30-7.27 (m, 1H), 6.82-6.80 (m, 1H), 6.78 (dd, 1H), 6.65 (dd, 1H), 4.45-4.25 (br m, 1H), 4.12-4.05 (m, 1H), 4.03-3.95 (m, 2H), 3.94-3.87 (m, 1H), 3.84 (s, 3H), 3.59 (br d, 1H), 1.44 (br s, 3H), 1.21 (br s, 3H). ESI-MS: 411.8 [M+H]⁺.

### Example 38

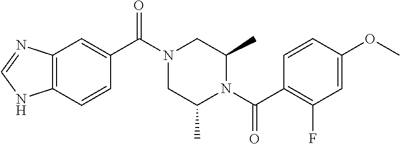 [SMILES: COc1ccc(C(=O)N2[C@H](C)CN(C(=O)c3ccc4[nH]cnc4c3)C[C@H]2C)c(F)c1]

((2R,6R)-4-(1H-Benzo[d]imidazole-5-carbonyl)-2,6-dimethylpiperazin-1-yl)(2-fluoro-4-methoxyphenyl)methanone (Compound 38)
The title compound was synthesized following the approach outlined in Procedure D substituting 1H-benzo[d]imidazole-5-carboxylic acid for 3-oxo-3,4-dihydro-2H-benzo[b][1,4]oxazine-6-carboxylic acid to afford the title compound (67.2 mg, 0.164 mmol, 55%) as a white solid. ESI-MS: 411.8 [M+H]⁺.
Procedure E: Example 39 ((3R,5R)-3,5-Dimethyl-4-(4-(oxazol-5-yl)benzoyl)piperazin-1-yl)(2-fluoro-4-methoxyphenyl)methanone (Compound 39)

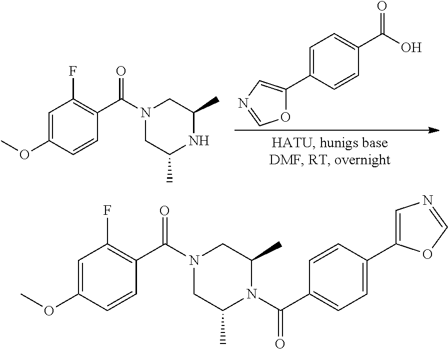

Into a 1 dram vial was added the ((3R,5R)-3,5-dimethylpiperazin-1-yl)(2-fluoro-4-methoxyphenyl)methanone (20 mg, 0.075 mmol), DMF (2.0 mL), and 4-(oxazol-5-yl)benzoic acid (0.09 mmol, 1.20 equiv). This was followed by addition of HATU (0.11 mmol), DIEA (0.225 mmol) and the resulting crude mixture stirred at 30° C. overnight. After such time the resulting mixture was filtered, concentrated and purified by Prep-HPLC directly to afford the title compound (18.8 mg, 0.043 mmol, 65%) as a white solid. ¹H-NMR (400 MHz, CDCl₃) δ 7.97 (s, 1H), 7.74 (d, 2H), 7.48 (d, 2H), 7.44 (s, 1H), 7.40 (dd, 1H), 6.79 (dd, 1H), 6.66 (dd, 1H), 4.45-4.36 (br m, 1H), 4.34-4.25 (br m, 1H), 4.01 (dd, 1H), 3.92 (br dd, 1H), 3.86 (s, 3H), 3.78 (dd, 1H), 3.33 (dd, 1H), 1.38 (d, 3H), 1.25 (d, 3H). ESI-MS: 438.4 [M+H]⁺.

### Example 40

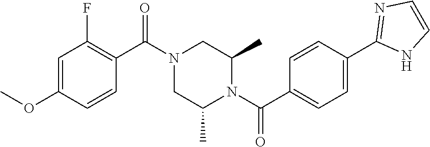 [SMILES: COc1ccc(C(=O)N2C[C@@H](C)N(C(=O)c3ccc(-c4ncc[nH]4)cc3)[C@H](C)C2)c(F)c1]

((3R,5R)-4-(4-(1H-imidazol-2-yl)benzoyl)-3,5-dimethylpiperazin-1-yl)(2-fluoro-4-methoxyphenyl)methanone (Compound 40)
The title compound was synthesized following the approach outlined in Procedure E substituting 4-(1H-imidazol-2-yl)benzoic acid for 4-(oxazol-5-yl)benzoic acid to afford the title compound (9.5 mg, 0.021 mmol, 33%) as a white solid. ¹H-NMR (400 MHz, CDCl₃) δ 7.74 (d, 2H), 7.32-7.28 (m, 1H), 7.26 (d, 2H), 7.06 (s, 2H), 6.70 (dd, 1H), 6.56 (dd, 1H), 4.40-4.28 (br m, 1H), 4.21-4.14 (br m, 1H), 3.92 (dd, 1H), 3.86-3.80 (m, 1H), 3.76 (s, 3H), 3.68 (br d, 1H), 3.24 (br d, 1H), 1.28 (br d, 3H), 1.14 (br d, 3H). ESI-MS: 437.8 [M+H]⁺.

### Example 41

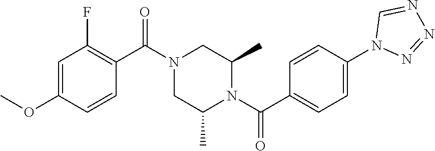 [SMILES: COc1ccc(C(=O)N2C[C@@H](C)N(C(=O)c3ccc(-n4cnnn4)cc3)[C@H](C)C2)c(F)c1]

((3R,5R)-4-(4-(1H-Tetrazol-1-yl)benzoyl)-3,5-dimethylpiperazin-1-yl)(2-fluoro-4-methoxyphenyl)methanone (Compound 41)
The title compound was synthesized following the approach outlined in Procedure E substituting 4-(1H-tetrazol-1-yl)benzoic acid for 4-(oxazol-5-yl)benzoic acid to afford the title compound (6.0 mg, 0.013 mmol, 21%) as a white solid. ESI-MS: 439.4 [M+H]⁺.

### Example 42

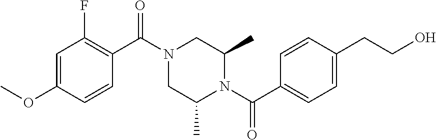 [SMILES: COc1ccc(C(=O)N2C[C@@H](C)N(C(=O)c3ccc(CCO)cc3)[C@H](C)C2)c(F)c1]

((2R,6R)-4-(2-Fluoro-4-methoxybenzoyl)-2,6-dimethylpiperazin-1-yl)(4-(2-hydroxyethyl)phenyl)methanone (Compound 42)
The title compound was synthesized following the approach outlined in Procedure E substituting 4-(2-hydroxyethyl)benzoic acid for 4-(oxazol-5-yl)benzoic acid to afford the title compound (6.6 mg, 0.016 mmol, 24%) as a white solid. ESI-MS: 415.7 [M+H]⁺.

### Example 43

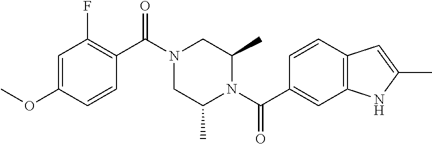

((3R,5R)-3,5-Dimethyl-4-(2-methyl-1H-indole-6-carbonyl)piperazin-1-yl)(2-fluoro-4-methoxyphenyl)methanone (Compound 43)
The title compound was synthesized following the approach outlined in Procedure E substituting 2-methyl-1H-indole-6-carboxylic acid for 4-(oxazol-5-yl)benzoic acid to afford the title compound (1.1 mg, 0.003 mmol, 4%) as a white solid. ESI-MS: 424.4 [M+H]⁺.

### Procedure F; Example 44

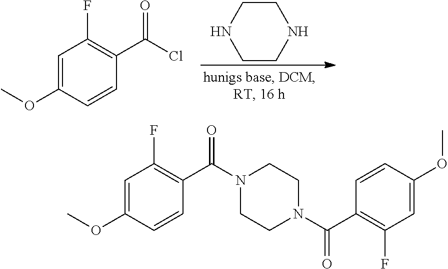

Piperazine-1,4-diylbis((2-fluoro-4-methoxyphenyl)methanone) (Compound 44)
To a 1 dram vial was added piperazine (9.14 mg, 0.087 mmol), DCM (2.6 mL) and hunig's base (46.3 μl, 0.265 mmol). After stirring for 10 mins at RT, 2-fluoro-4-methoxybenzoyl chloride (50 mg, 0.265 mmol) was added to the crude reaction mixture and the reaction stirred at RT for 16h. After such time the reaction was concentrated and purified by reverse phase HPLC to afford the title compound (42 mg, 0.107 mmol, 41%) as a white solid. ¹H-NMR (400 MHz, CDCl₃) δ 7.38 (dd, 2H), 6.81-6.75 (m, 2H), 6.68-6.58 (m, 2H), 3.91-3.79 (m, 4H), 3.86 (s, 6H), 3.50-3.36 (m, 4H). ESI-MS: 391.2 [M+H]⁺.

### Example 45

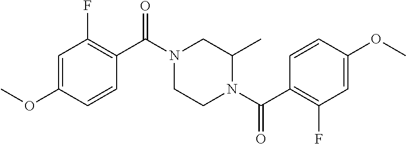

(2-Methylpiperazine-1,4-diyl)bis((2-fluoro-4-methoxyphenyl)methanone) (Compound 45)
The title compound was synthesized following the approach outlined in Procedure F substituting 2-methylpiperazine for piperazine to afford the title compound (20.8 mg, 0.051 mmol, 48%) as a white solid. ESI-MS: 405.6 [M+H]⁺.

### Example 46

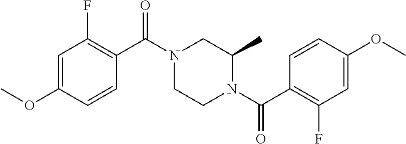

(R)-(2-methylpiperazine-1,4-diyl)bis((2-fluoro-4-methoxyphenyl)methanone) (Compound 46)
The title compound was synthesized following the approach outlined in Procedure F substituting (R)-2-methylpiperazine for piperazine to afford the title compound (30.6 mg, 0.075 mmol, 43%) as a white solid. ESI-MS: 405.6 [M+H]⁺.

### Example 47

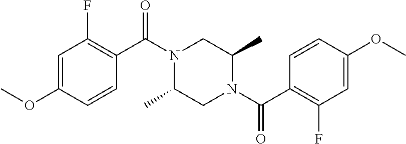

trans-2,5-Dimethylpiperazine-1,4-diyl)bis((2-fluoro-4-methoxyphenyl)methanone) (Compound 47)
The title compound was synthesized following the approach outlined in Procedure F substituting trans-2,5-dimethylpiperazine for piperazine to afford the title compound (11.0 mg, 0.026 mmol, 25%) as a white solid. ESI-MS: 419.7 [M+H]⁺.

### Example 48

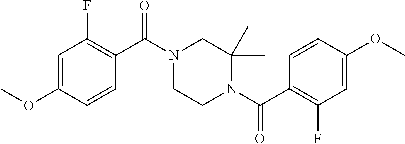

(2,2-Dimethylpiperazine-1,4-diyl)bis((2-fluoro-4-methoxyphenyl)methanone) (Compound 48)
The title compound was synthesized following the approach outlined in Procedure F substituting 2,2-dimethylpiperazine for piperazine to afford the title compound (10.5 mg, 0.024 mmol, 25%) as a white solid. ESI-MS: 419.7 [M+H]⁺.

### Example 49

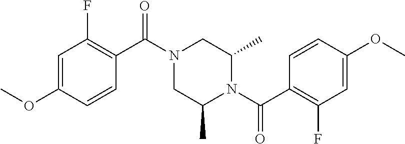

((2S,6S)-2,6-Dimethylpiperazine-1,4-diyl)bis((2-fluoro-4-methoxyphenyl)methanone) (Compound 49)
The title compound was synthesized following the approach outlined in Procedure F substituting (2S,6S)-2,6-dimethylpiperazine for piperazine to afford the title compound (80.4 mg, 0.576 mmol, 69%) as a white solid. ¹H-NMR (400 MHz, DMSO-d6) δ 7.36 (dt, 2H), 6.95 (dd, 1H), 6.92 (dd, 1H), 6.89-6.84 (m, 2H), 3.90-3.84 (br d, 1H), 3.82-3.77 (m, 1H), 3.81 (s, 3H), 3.80 (s, 3H), 3.64-3.57 (m, 1H), 3.34-3.29 (m, 2H), 3.27-3.22 (d, 1H), 1.23 (br s, 3H), 1.08 (br s, 3H). ESI-MS: 419.7 [M+H]⁺.

### Example 50

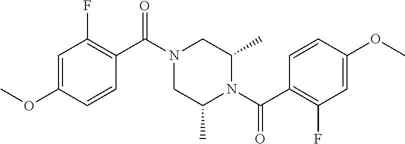

((2R,6S)-2,6-Dimethylpiperazine-1,4-diyl)bis((2-fluoro-4-methoxyphenyl)methanone) (Compound 50)
The title compound was synthesized following the approach outlined in Procedure F substituting (2R,6S)-2,6-dimethylpiperazine for piperazine to afford the title compound (22.9 mg, 0.055 mmol, 3%) as a white solid. ESI-MS: 419.7 [M+H]⁺.

### Example 51

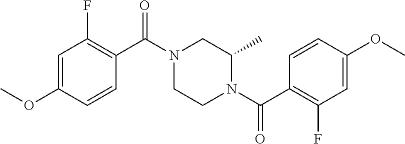

(S)-(2-Methylpiperazine-1,4-diyl)bis((2-fluoro-4-methoxyphenyl)methanone) (Compound 51)
The title compound was synthesized following the approach outlined in Procedure F substituting (S)-2-methylpiperazine for piperazine to afford the title compound (27.9 mg, 0.069 mmol, 39%) as a white solid. ESI-MS: 405.7 [M+H]⁺.

### Example 52

Procedure G: ((2R,6R)-4-(1H-indole-6-carbonyl)-2,6-dimethylpiperazin-1-yl)(4,7-dimethoxy-1H-indol-2-yl)methanone (Compound 52).

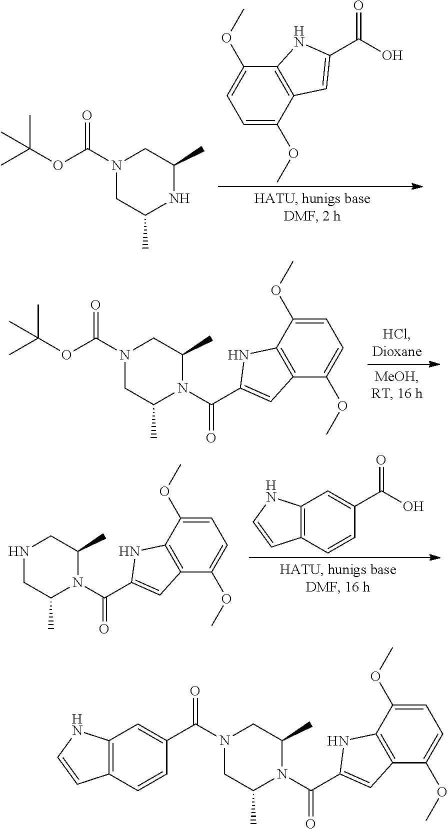

a. tert-Butyl (3R,5R)-4-(4,7-dimethoxy-1H-indole-2-carbonyl)-3,5-dimethylpiperazine-1-carboxylate

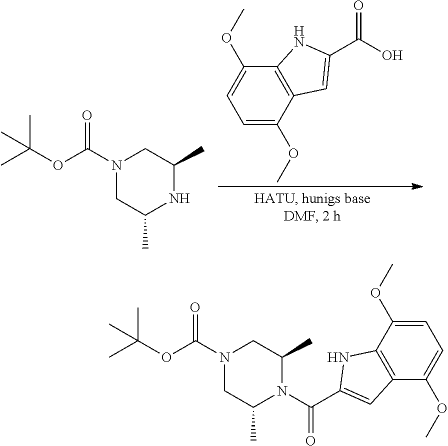 [SMILES: C[C@@H]1CN(C(=O)OC(C)(C)C)C[C@@H](C)N1 ; COc1ccc(OC)c2[nH]c(C(=O)O)cc12 ; COc1ccc(OC)c2[nH]c(C(=O)N3[C@H](C)CN(C(=O)OC(C)(C)C)C[C@H]3C)cc12]

Into a 8-mL vial, was added 4,7-dimethoxy-1H-indole-2-carboxylic acid (80.0 mg, 0.362 mmol), tert-butyl (3R,5R)-3,5-dimethylpiperazine-1-carboxylate (77.5 mg, 0.362 mmol), HATU (151.2 mg, 0.398 mmol), DIEA (1.08 mmol), and DMF (3.0 mL, 0.041 mmol) and the resulting solution was stirred for 2 hr at 25° C. After such time the resulting mixture was concentrated under vacuum, the crude material purified by flash chromatography on silica eluting with DCM:MeOH (20:1) to afford the title compound (107 mg, 71%) as a white solid. ESI-MS: 418.1 [M+H]⁺.
b. (4,7-dimethoxy-1H-indol-2-yl)((2R,6R)-2,6-dimethylpiperazin-1-yl)methanone.

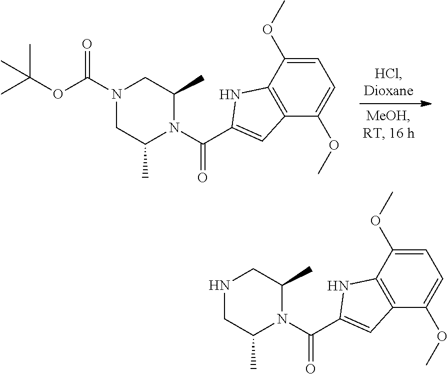 [SMILES: COc1ccc(OC)c2[nH]c(C(=O)N3[C@H](C)CN(C(=O)OC(C)(C)C)C[C@H]3C)cc12 ; COc1ccc(OC)c2[nH]c(C(=O)N3[C@H](C)CNC[C@H]3C)cc12]

Into an 8-mL vial was added HCl in 1,4-dioxane (3.0 mL, 98.7 mmol), DCM (3.0 mL, 47.19 mmol), and tert-butyl (3R,5R)-4-(4,7-dimethoxy-1H-indole-2-carbonyl)-3,5-dimethylpiperazine-1-carboxylate (79.0 mg, 0.189 mmol). The resulting solution was stirred for 1 hr at 25° C., after which time the crude material was concentrated under vacuum and purified by flash chromatography on silica eluting with chloroform/methanol (20/1)) to afford the title compound (67 mg) as a white solid. ESI-MS: 318.4 [M+H]⁺.
c. ((2R,6R)-4-(1H-indole-6-carbonyl)-2,6-dimethylpiperazin-1-yl)(4,7-dimethoxy-1H-indol-2-yl)methanone.

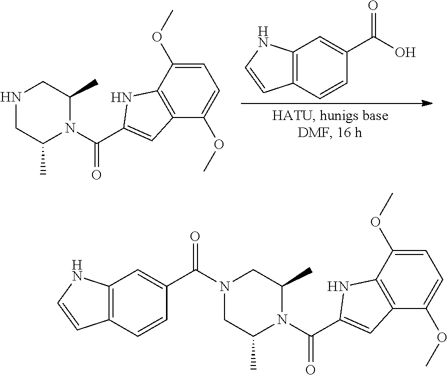

Into a 40-mL vial, was placed 1-[(3R,5R)-3,5-dimethylpiperazin-1-yl]-2-(1H-indol-2-yl)ethanone (58.0 mg, 0.214 mmol), 4,7-dimethoxy-1H-indole-2-carboxylic acid (47.3 mg, 0.214 mmol), HATU (97.5 mg, 0.256 mmol), DMF (3.0 mL, 38.76 mmol), and DIEA (0.641 mmol). The resulting solution was stirred for 16 hr at 25° C., after which time the crude material was concentrated and purified by Prep-HPLC to afford the title compound (37.4 mg, 0.078 mmol, 36.8%) as a white solid. ¹H-NMR (400 MHz, DMSO-d6, 350K) 6 11.06 (br s, 1H), 10.92 (br s, 1H), 7.61 (d, 1H, J=8.16 Hz), 7.55 (s, 1H), 7.45-7.43 (m, 1H), 7.15-7.12 (m, 1H), 6.78 (d, 1H, J=1.88 Hz), 6.63 (d, 1H, J=8.28 Hz), 6.5 (br s, 1H), 6.42 (d, 1H, J=8.28 Hz), 4.56-4.50 (m, 2H), 3.90-3.83 (br m, 2H), 3.88 (s, 3H), 3.85 (br s, 3H), 3.68 (br d, 2H, J=11.05 Hz), 1.33 (s, 3H), 1.32 (s, 3H). ESI-MS: 461.5 [M+H]⁺.

Activity Testing

Methods

Purification of CPS1 protein

CPS1 was expressed and purified from SF21 insect cells. The full length, human CPS1 gene was cloned into pFastBac HTA and baculovirus was generated using the Bac-to-bac expression system from Invitrogen. P3 virus was used to infect SF21 insect cells at a density of 1.5E6 cells/mL at an MOI of 2. Cells were harvested by centrifugation after 60 hrs and the cell pellet resuspended in lysis buffer (50 mM glycyl glycine pH 7.4, 20 mM KCl, 1 mM TCEP, 10% glycerol and 20 mM imidazole with Roche protease inhibitor tablet) before being lysed with 40 strokes of a Dounce homogenizer on ice. The insoluble fraction of the lysate was then cleared by centrifugation at 30,000 rpm for 30 min. 40 mL of supernatant was mixed with 2 mL of Ni NTA beads for 45 min at 4° C. in a 50 mL conical tube.

The beads were washed with 40 mL of wash buffer (50 mM glycyl glycine pH 7.4, 500 mM NaCl, 1 mM TCEP, 10% glycerol and 50 mM imidazole) by centrifugation and resuspension of the beads three times. Bound CPS1 protein was eluted in 6 mL of elution buffer (50 mM glycyl glycine pH 7.4, 500 mM NaCl, 1 mM TCEP, 10% glycerol and 250 mM imidazole) three times. Elution fractions were combined and concentrated using an Amicon Ultra centrifugation filter with a 10,000 molecular weight cut off. Imidazole was removed by repeated concentration and resuspension in storage buffer in the filter device (50 mM glycyl glycine pH 7.4, 500 mM NaCl, 1 mM TCEP and 20% glycerol). CPS1 was concentrated to a final concentration of ˜3 mg/mL before being aliquoted, flash frozen in liquid nitrogen and stored at −80° C.

Detection of CPS1 Activity and Compound Profiling

CPS1 activity was measured and compounds profiled in an endpoint assay measuring the ATPase activity of CPS1 using the ADP-Glo kit from Promega. 50 nM CPS1 was incubated alone or with varying amounts of tested compound from DMSO stocks in assay buffer (50 mM HEPES pH 7.0, 12.5 mM KHCO₃, 1.5 mM (NH₄)₂SO₃, 5 mM MgCl₂, 50 mM KCl, 1 mM TCEP, 0.01% TritonX 100, 0.0025% BSA and 0.2 mM NAG) for 20 min in a 3 μL volume in a 384-well plate (Corning Cat#3820). The final concentration of DMSO was 0.04%. The reaction was initiated by the addition of 10 μM ATP, bringing the final volume to 6 μL. After 60 min at room temperature, the reaction was terminated with 3 μL ADP-Glo reagent and incubated at room temperature for 30 min. 3 μL of kinase detection reagent was then added and luminescent signal measured on an Envision plate reader after 30 min.

Results are Shown in Table 2, Above.

Assay 2-Transcreener ADP FP Assay

ADP formation from CPS1 was measured in an endpoint assay using the Transcreener ADP FP kit from BellBbrook (Cat #3010-1K). 10 nM CPS1 was incubated with varying amount of inhibitor in assay buffer (50 mM HEPES pH 7.0, 12.5 mM KHCO3, 1.5 mM (NH4)₂SO3, 5 mM MgCl₂, 50 mM KCl, 1 mM TCEP, 0.01% TritonX 100, 0.0025% BSA and 0.2 mM for 20 min. The reaction was initiated with varying amounts of ATP in a 384-well plate with a final volume of 6 μL. After 60 min the reaction was quenched with 6 uL of 30 mM EDTA. ADP Alexa Fluor 633 and detection antibody were added at a final concentration of 2 nM and 6 μg/mL, respectively. Fluorescence polarization (FP) was measured 30 min later on a Tecan M1000 plate reader. Compound 108 was found to have an FP (CPS1) IC50 (nM) value of >66,666 using this assay.

New, improved, and nonobvious compositions have been described in this specification with sufficient particularity as to be understood by one of ordinary skill in the art. Moreover, it will be apparent to those skilled in the art that modifications, variations, substitutions, and equivalents exist for features of the compositions which do not materially depart from the spirit and scope of the embodiments disclosed herein. Accordingly, it is expressly intended that all such modifications, variations, substitutions, and equivalents which fall within the spirit and scope of the invention as defined by the appended claims shall be embraced by the appended claims.

1. A compound given by Formula I 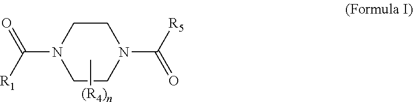
or a pharmaceutically acceptable salt thereof, wherein
R₁ is selected from the group consisting of 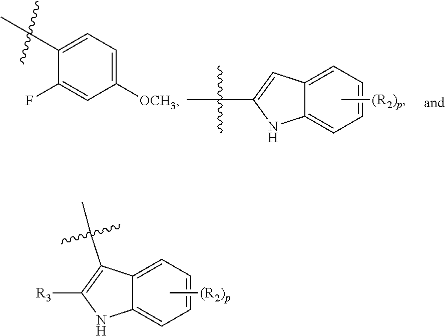
H Wherein
R₂ are individually the same or different and are selected from the group consisting of—OCH₃, —Cl, —OH, —CH₃, and —F;
R₃ are individually the same or different and are selected from the group consisting of—H or —CH₃;
n is 0-2;
R₄ are individually the same or different and are selected from —H or —CH₃;
R₅ is selected from the group consisting of: 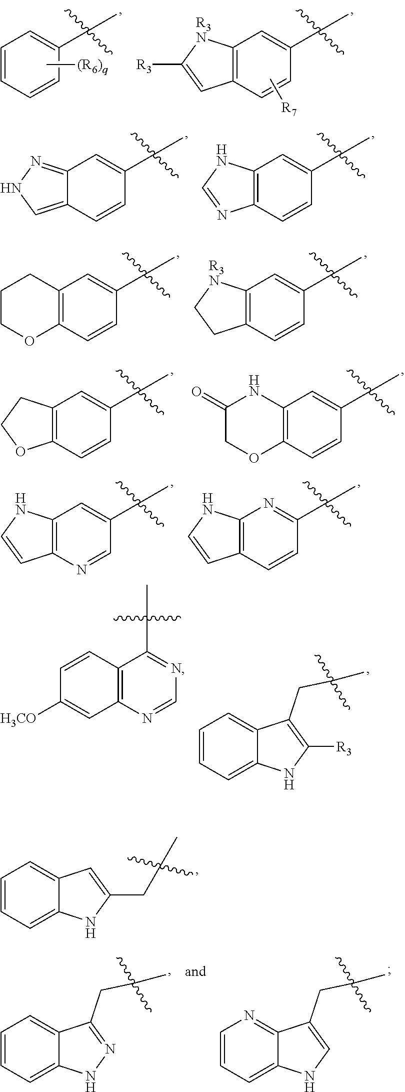
wherein
q is 0-3;
R₅ are independently the same or different and are selected from the group consisting of—F, —OCH₃, —OCHF₂, —SCH₃, —CH₃, —OCF₃, —OCH₂Ph, —OCH(CH₃)₂, —NHC(O)CH₃, —Cl, —OH, —N(CH₃)₂, —OEt, —CH₂CH₂OH, —S(O)₂CH₃, 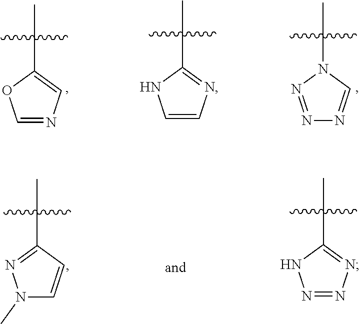
R₇ is —H, —F or —Cl.

2. The compound or pharmaceutically acceptable salt of claim 1, wherein R₁ is

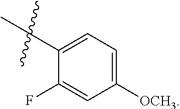 [SMILES: *c1ccc(OC)cc1F]

3. The compound or pharmaceutically acceptable salt according to claim 2,
wherein R₅ is selected from the group consisting of: 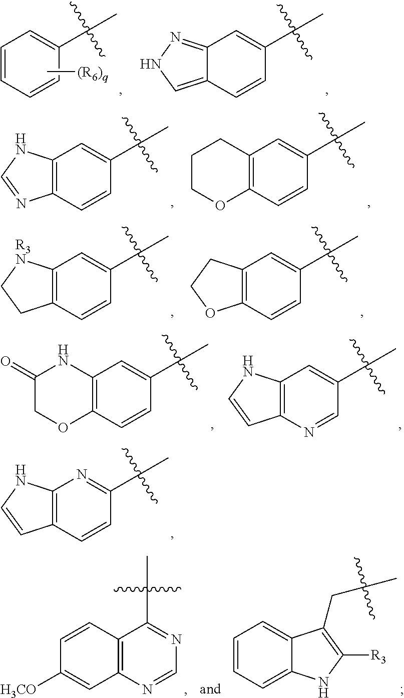
wherein
R₆ are independently the same or different and are selected from the group consisting of—F, —OCH₃, —OCHF₂, —SCH₃, —CH₃, —OCF₃, —OCH₂Ph, —OCH(CH₃)₂, —Cl, —OH, —N(CH₃)₂, —OEt, —CH₂CH₂OH, 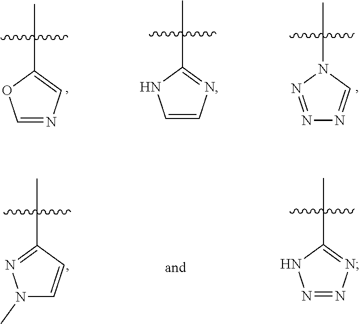
wherein only one R₅ may be —OCH₃; and
R₇ is —H, —F or —Cl.

4. The compound or pharmaceutically acceptable salt of claim 3, given by Formula II or Formula III 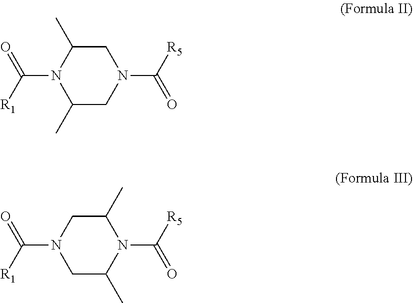
or a pharmaceutically acceptable salt thereof, wherein R₅ is selected from the group consisting of: 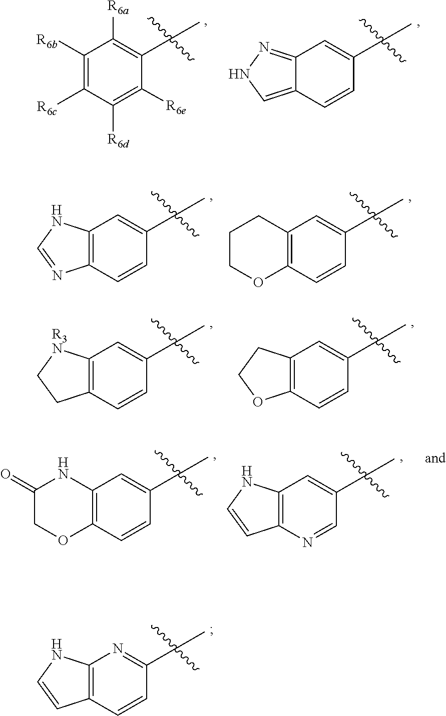
wherein
R₆ₐ is selected from the group consisting of—H, and —F;
R_{6b} is selected from the group consisting of—H, and —F;
R_{6c} is selected from the group consisting of—EtOH, —OCH₃, —Cl, —N(CH₃)₂, —OH, —F, —OCHF₂, —CH₃, —OCH(CH₃)₂, —OCF₃, —OCH₂Ph, —OEt, —SCH₃, 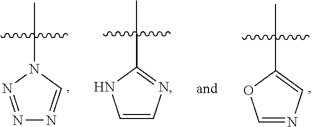 [SMILES: *n1cnnn1 ; *c1ncc[nH]1 ; *c1cnco1]
R_{6d} is selected from the group consisting of—H, and —F;
R₆ₑ is selected from the group consisting of—H, and —F;
wherein at least two of R₆ₐ, R_{6b}, R_{6d} or R₆ₑ are —H.

5. The compound or pharmaceutically acceptable salt of claim 4, wherein R₅ is

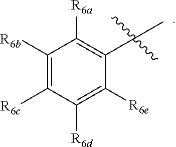

6. The compound or pharmaceutically acceptable salt of claim 1, wherein R₁ is selected from the group consisting of 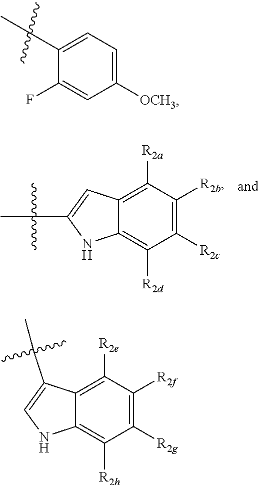
R₂ₐ is —H or —OCH₃;
R_{2b} is —H or —OH;
R₂ₑ is —H;
R_{2d} is —H or —OCH₃;
R₂ₑ is —H;
R_{2f} is —H, —F or —Cl;
R_{2g} is —H; and
R₂ₕ is —H, —F, —Cl or —CH₃.

7. The compound or pharmaceutically acceptable salt according to claim 6,
wherein R₁ is 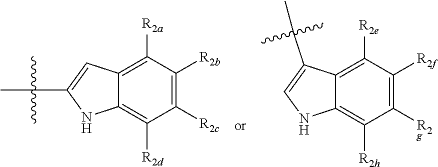
and wherein R₅ is selected from the group consisting of

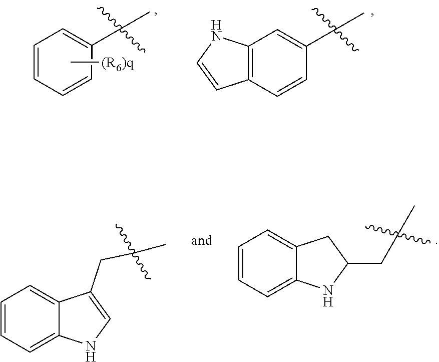

8. The compound or pharmaceutically acceptable salt of claim 7, wherein R₅ is selected from the group consisting of and

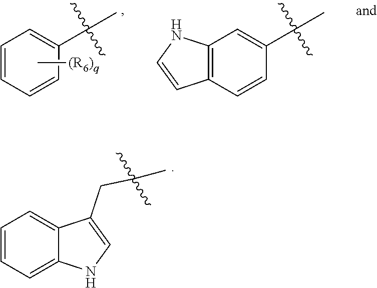

9. The compound or pharmaceutically acceptable salt of claim 7, wherein R₅ is

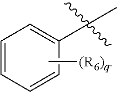

10. The compound or pharmaceutically acceptable salt of claim 7, wherein R₅ is 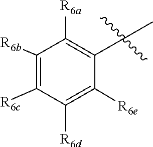
wherein
R₆ₐ is selected from the group consisting of—H, and —F;
R_{6b} is selected from the group consisting of—H, and —F;
R_{6c} is selected from the group consisting of—EtOH, —OCH₃, —Cl, —N(CH₃)₂, —OH, —F, —OCHF₂, —CH₃, —OCH(CH₃)₂, —OCF₃, —OCH₂Ph, —OEt, —SCH₃, 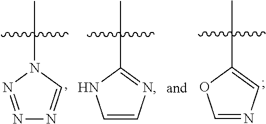
R_{6d} is selected from the group consisting of—H, and —F;
R₆ₑ is selected from the group consisting of—H, and —F;
wherein at least two of R₆ₐ, R_{6b}, R_{6d} or R₆ₑ are —H.

11. A compound selected from the group consisting of:
((2R,6R)-4-(2-fluoro-4-(trifluoromethoxy)benzoyl)-2,6-dimethylpiperazin-1-yl)(2-fluoro-4-methoxyphenyl)methanone;
((2R,6R)-4-(2,3-difluoro-4-methoxybenzoyl)-2,6-dimethylpiperazin-1-yl)(2-fluoro-4-methoxyphenyl)methanone;
((2R,6R)-4-(1H-indole-6-carbonyl)-2,6-dimethylpiperazin-1-yl)(2-fluoro-4-methoxyphenyl)methanone;
((2R,6R)-4-(3-fluoro-4-methoxybenzoyl)-2,6-dimethylpiperazin-1-yl)(2-fluoro-4-methoxyphenyl)methanone;
((2R,6R)-4-(4-ethoxy-2-fluorobenzoyl)-2,6-dimethylpiperazin-1-yl)(2-fluoro-4-methoxyphenyl)methanone;
((2R,6R)-2,6-dimethyl-4-(1-methylindoline-6-carbonyl)piperazin-1-yl)(2-fluoro-4-methoxyphenyl)methanone;
((2R,6R)-2,6-dimethyl-4-(2-methyl-1H-indole-6-carbonyl)piperazin-1-yl)(2-fluoro-4-methoxyphenyl)methanone;
N-(3-fluoro-4-((3R,5R)-4-(2-fluoro-4-methoxybenzoyl)-3,5-dimethylpiperazine-1-carbonyl)phenyl)acetamide;
((2R,6R)-4-(4-(difluoromethoxy)-2-fluorobenzoyl)-2,6-dimethylpiperazin-1-yl)(2-fluoro-4-methoxyphenyl)methanone;
((2R,6R)-4-(2,5-difluoro-4-methoxybenzoyl)-2,6-dimethylpiperazin-1-yl)(2-fluoro-4-methoxyphenyl)methanone;
((2R,6R)-4-(2-chloro-4-methoxybenzoyl)-2,6-dimethylpiperazin-1-yl)(2-fluoro-4-methoxyphenyl)methanone;
((2R,6R)-4-(2,4-difluorobenzoyl)-2,6-dimethylpiperazin-1-yl)(2-fluoro-4-methoxyphenyl)methanone;
((2R,6R)-4-(2H-indazole-6-carbonyl)-2,6-dimethylpiperazin-1-yl)(2-fluoro-4-methoxyphenyl)methanone;
((3R,5R)-4-(2-fluoro-4-methoxybenzoyl)-3,5-dimethylpiperazin-1-yl)(2-fluoro-4-methylphenyl)methanone;
((2R,6R)-4-(2,6-difluoro-4-methoxybenzoyl)-2,6-dimethylpiperazin-1-yl)(2-fluoro-4-methoxyphenyl)methanone;
((2R,6R)-4-(2,6-difluoro-4-(methylthio)benzoyl)-2,6-dimethylpiperazin-1-yl)(2-fluoro-4-methoxyphenyl)methanone;
(4-chloro-1H-indol-6-yl)((3R,5R)-4-(2-fluoro-4-methoxybenzoyl)-3,5-dimethylpiperazin-1-yl)methanone;
((2R,6R)-4-(chroman-6-carbonyl)-2,6-dimethylpiperazin-1-yl)(2-fluoro-4-methoxyphenyl)methanone;
((2R,6R)-2,6-dimethyl-4-(1H-pyrrolo[2,3-b]pyridine-6-carbonyl)piperazin-1-yl)(2-fluoro-4-methoxyphenyl)methanone;
((2R,6R)-4-(4-(dimethylamino)-2-fluorobenzoyl)-2,6-dimethylpiperazin-1-yl)(2-fluoro-4-methoxyphenyl)methanone;
((2R,6R)-4-(2,3-dihydrobenzofuran-5-carbonyl)-2,6-dimethylpiperazin-1-yl)(2-fluoro-4-methoxyphenyl)methanone;
((2R,6R)-4-(2-fluoro-4-methoxybenzoyl)-2,6-dimethylpiperazin-1-yl)(2-fluoro-4-methylphenyl)methanone;
((3R,5R)-4-(2,5-difluoro-4-methoxybenzoyl)-3,5-dimethylpiperazin-1-yl)(2-fluoro-4-methoxyphenyl)methanone;
((3R,5R)-4-(4-(benzyloxy)-2-fluorobenzoyl)-3,5-dimethylpiperazin-1-yl)(2-fluoro-4-methoxyphenyl)methanone;
((3R,5R)-4-(2,6-difluoro-4-(methylthio)benzoyl)-3,5-dimethylpiperazin-1-yl)(2-fluoro-4-methoxyphenyl)methanone;
((2R,6R)-2,6-dimethylpiperazine-1,4-diyl)bis((2-fluoro-4-methoxyphenyl)methanone);
((3R,5R)-4-(4-ethoxy-2-fluorobenzoyl)-3,5-dimethylpiperazin-1-yl)(2-fluoro-4-methoxyphenyl)methanone;
((3R,5R)-4-(2-fluoro-4-methoxy-6-methylbenzoyl)-3,5-dimethylpiperazin-1-yl)(2-fluoro-4-methoxyphenyl)methanone;
((2R,6R)-4-(2-fluoro-4-methoxybenzoyl)-2,6-dimethylpiperazin-1-yl)(3-fluoro-4-methoxyphenyl)methanone;
((3R,5R)-4-(2-fluoro-4-isopropoxybenzoyl)-3,5-dimethylpiperazin-1-yl)(2-fluoro-4-methoxyphenyl)methanone;
((3R,5R)-4-(2-fluoro-4-(trifluoromethoxy)benzoyl)-3,5-dimethylpiperazin-1-yl)(2-fluoro-4-methoxyphenyl)methanone;
((3R,5R)-4-(2,4-difluorobenzoyl)-3,5-dimethylpiperazin-1-yl)(2-fluoro-4-methoxyphenyl)methanone;
((3R,5R)-4-(4-(difluoromethoxy)-2-fluorobenzoyl)-3,5-dimethylpiperazin-1-yl)(2-fluoro-4-methoxyphenyl)methanone;
((3R,5R)-4-(2,3-difluoro-4-methoxybenzoyl)-3,5-dimethylpiperazin-1-yl)(2-fluoro-4-methoxyphenyl)methanone;
6-((3R,5R)-4-(2-fluoro-4-methoxybenzoyl)-3,5-dimethylpiperazine-1-carbonyl)-2H-benzo[b][1,4]oxazin-3(4H)-one;
(7-chloro-1H-indol-3-yl)((3R,5R)-4-(2-fluoro-4-methoxybenzoyl)-3,5-dimethylpiperazin-1-yl)methanone;
((2R,6R)-2,6-dimethyl-4-(1H-pyrrolo[3,2-b]pyridine-6-carbonyl)piperazin-1-yl)(2-fluoro-4-methoxyphenyl)methanone;
((2R,6R)-4-(1H-benzo[d]imidazole-6-carbonyl)-2,6-dimethylpiperazin-1-yl)(2-fluoro-4-methoxyphenyl)methanone;
((3R,5R)-3,5-dimethyl-4-(4-(oxazol-5-yl)benzoyl)piperazin-1-yl)(2-fluoro-4-methoxyphenyl)methanone;
((3R,5R)-4-(4-(1H-imidazol-2-yl)benzoyl)-3,5-dimethylpiperazin-1-yl)(2-fluoro-4-methoxyphenyl)methanone;
((3R,5R)-4-(4-(1H-tetrazol-1-yl)benzoyl)-3,5-dimethylpiperazin-1-yl)(2-fluoro-4-methoxyphenyl)methanone;
((2R,6R)-4-(2-fluoro-4-methoxybenzoyl)-2,6-dimethylpiperazin-1-yl)(4-(2-hydroxyethyl)phenyl)methanone;
((3R,5R)-3,5-dimethyl-4-(2-methyl-1H-indole-6-carbonyl)piperazin-1-yl)(2-fluoro-4-methoxyphenyl)methanone;
piperazine-1,4-diylbis((2-fluoro-4-methoxyphenyl)methanone);
(2-methylpiperazine-1,4-diyl)bis((2-fluoro-4-methoxyphenyl)methanone);
(R)-(2-methylpiperazine-1,4-diyl)bis((2-fluoro-4-methoxyphenyl)methanone);
((2S,5R)-2,5-dimethylpiperazine-1,4-diyl)bis((2-fluoro-4-methoxyphenyl)methanone);
(2,2-dimethylpiperazine-1,4-diyl)bis((2-fluoro-4-methoxyphenyl)methanone);
((2S,6S)-2,6-dimethylpiperazine-1,4-diyl)bis((2-fluoro-4-methoxyphenyl)methanone);
((2S,6R)-2,6-dimethylpiperazine-1,4-diyl)bis((2-fluoro-4-methoxyphenyl)methanone);
(S)-(2-methylpiperazine-1,4-diyl)bis((2-fluoro-4-methoxyphenyl)methanone);
((2R,6R)-4-(1H-indole-6-carbonyl)-2,6-dimethylpiperazin-1-yl)(4,7-dimethoxy-1H-indol-2-yl)methanone;
((3R,5R)-4-(1H-indole-6-carbonyl)-3,5-dimethylpiperazin-1-yl)(4,7-dimethoxy-1H-indol-2-yl)methanone;
(4-(1H-indole-6-carbonyl)piperazin-1-yl)(4,7-dimethoxy-1H-indol-2-yl)methanone;
((2R,6R)-4-(2-fluoro-4-methoxybenzoyl)-2,6-dimethylpiperazin-1-yl)(5-hydroxy-1H-indol-2-yl)methanone;
((2R,6R)-2,6-dimethyl-4-(7-methyl-1H-indole-3-carbonyl)piperazin-1-yl)(2-fluoro-4-methoxyphenyl)methanone;
((3R,5R)-4-(1H-indole-2-carbonyl)-3,5-dimethylpiperazin-1-yl)(2-fluoro-4-methoxyphenyl)methanone;
(7-fluoro-1H-indol-3-yl)((3R,5R)-4-(2-fluoro-4-methoxybenzoyl)-3,5-dimethylpiperazin-1-yl)methanone;
((2R,6R)-4-(1H-indole-2-carbonyl)-2,6-dimethylpiperazin-1-yl)(2-fluoro-4-methoxyphenyl)methanone;
(2-aminothiazolidin-5-yl)((3R,5R)-4-(2-fluoro-4-methoxybenzoyl)-3,5-dimethylpiperazin-1-yl)methanone;
(5-chloro-1H-indol-3-yl)((3R,5R)-4-(2-fluoro-4-methoxybenzoyl)-3,5-dimethylpiperazin-1-yl)methanone;
(2-fluoro-4-methoxyphenyl)((2R,6R)-4-(7-methoxyquinazolin-4-yl)-2,6-dimethylpiperazin-1-yl)methanone;
((2R,6R)-2,6-dimethylpiperazine-1,4-diyl)bis((4-methoxyphenyl)methanone);
1-(4-(4,7-dimethoxy-1H-indole-2-carbonyl)piperazin-1-yl)-2-(1H-indol-3-yl)ethanone;
((2R,6R)-4-(2-fluoro-4-hydroxybenzoyl)-2,6-dimethylpiperazin-1-yl)(2-fluoro-4-methoxyphenyl)methanone;
((3R,5R)-4-(4-chloro-2-fluorobenzoyl)-3,5-dimethylpiperazin-1-yl)(2-fluoro-4-methoxyphenyl)methanone;
((2R,6R)-4-(2-fluoro-4-methoxybenzoyl)-2,6-dimethylpiperazin-1-yl)(2-fluoro-5-methoxyphenyl)methanone;
(2,6-dimethylpiperazine-1,4-diyl)bis((2-fluoro-4-methoxyphenyl)methanone);
((3R,5R)-4-(2-fluoro-4-methoxybenzoyl)-3,5-dimethylpiperazin-1-yl)(indolin-6-yl)methanone;
((2R,6R)-4-(2-fluoro-4-methoxybenzoyl)-2,6-dimethylpiperazin-1-yl)(3-hydroxy-4-methylphenyl)methanone;
(2,5-dimethylpiperazine-1,4-diyl)bis((2-fluoro-4-methoxyphenyl)methanone);
((3R,5R)-4-(2-fluoro-4-methoxybenzoyl)-3,5-dimethylpiperazin-1-yl)(3-hydroxy-4-methoxyphenyl)methanone;
(7-fluoro-1H-indol-6-yl)((2R,6R)-4-(2-fluoro-4-methoxybenzoyl)-2,6-dimethylpiperazin-1-yl)methanone;
((3R,5R)-4-(chroman-6-carbonyl)-3,5-dimethylpiperazin-1-yl)(2-fluoro-4-methoxyphenyl)methanone;
((3R,5R)-4-(2-fluoro-3-hydroxybenzoyl)-3,5-dimethylpiperazin-1-yl)(2-fluoro-4-methoxyphenyl)methanone;
((3R,5R)-4-(2-fluoro-4-(methylsulfonyl)benzoyl)-3,5-dimethylpiperazin-1-yl)(2-fluoro-4-methoxyphenyl)methanone;
((2R,6R)-4-(1H-indole-3-carbonyl)-2,6-dimethylpiperazin-1-yl)(2-fluoro-4-methoxyphenyl)methanone;
1-((3R,5R)-4-(2-fluoro-4-methoxybenzoyl)-3,5-dimethylpiperazin-1-yl)-2-(2-methyl-1H-indol-3-yl)ethanone;
((2R,6R)-4-(2-fluoro-4-methoxybenzoyl)-2,6-dimethylpiperazin-1-yl)(4-methoxy-3-methylphenyl)methanone;
((3R,5R)-3,5-dimethyl-4-(4-(1-methyl-1H-pyrazol-3-yl)benzoyl)piperazin-1-yl)(2-fluoro-4-methoxyphenyl)methanone;
((2R,6R)-4-(2-fluoro-4-methoxy-6-methylbenzoyl)-2,6-dimethylpiperazin-1-yl)(2-fluoro-4-methoxyphenyl)methanone;
(4,7-dimethoxy-1H-indol-2-yl)((3R,5R)-4-(2-fluoro-4-methoxybenzoyl)-3,5-dimethylpiperazin-1-yl)methanone;
((2R,6R)-2,6-dimethyl-4-(1-methyl-1H-indole-6-carbonyl)piperazin-1-yl)(2-fluoro-4-methoxyphenyl)methanone;
1-((3R,5R)-4-(4,7-dimethoxy-1H-indole-2-carbonyl)-3,5-dimethylpiperazin-1-yl)-2-(1H-indol-2-yl)ethanone;
((2R,6R)-4-(2-fluoro-4-methoxybenzoyl)-2,6-dimethylpiperazin-1-yl)(3-hydroxyphenyl)methanone;
(2,3-dimethylpiperazine-1,4-diyl)bis((2-fluoro-4-methoxyphenyl)methanone);
(5-fluoro-1H-indol-3-yl)((3R,5R)-4-(2-fluoro-4-methoxybenzoyl)-3,5-dimethylpiperazin-1-yl)methanone;
1-((3R,5R)-4-(2-fluoro-4-methoxybenzoyl)-3,5-dimethylpiperazin-1-yl)-2-(1H-indazol-3-yl)ethanone;
1-((3R,5R)-4-(2-fluoro-4-methoxybenzoyl)-3,5-dimethylpiperazin-1-yl)-2-(1H-pyrrolo[3,2-b]pyridin-3-yl)ethanone;
((3R,5R)-4-(4-(1H-tetrazol-5-yl)benzoyl)-3,5-dimethylpiperazin-1-yl)(2-fluoro-4-methoxyphenyl)methanone;
1-((2R,6R)-4-(4,7-dimethoxy-1H-indole-2-carbonyl)-2,6-dimethylpiperazin-1-yl)-2-(1H-indol-2-yl)ethanone;
((2R,6R)-4-(2-fluoro-3-methoxybenzoyl)-2,6-dimethylpiperazin-1-yl)(2-fluoro-4-methoxyphenyl)methanone;
N-(3-fluoro-4-((2R,6R)-4-(2-fluoro-4-methoxybenzoyl)-2,6-dimethylpiperazine-1-carbonyl)phenyl)acetamide;
((3R,5R)-4-(2-chloro-4-methoxybenzoyl)-3,5-dimethylpiperazin-1-yl)(2-fluoro-4-methoxyphenyl)methanone;
((3R,5R)-4-(2,6-difluoro-4-methoxybenzoyl)-3,5-dimethylpiperazin-1-yl)(2-fluoro-4-methoxyphenyl)methanone;
((3R,5R)-4-(2-fluoro-4-methoxybenzoyl)-3,5-dimethylpiperazin-1-yl)(2-fluoro-5-methoxyphenyl)methanone;
((3S,5S)-4-(2-fluoro-4-methoxybenzoyl)-3,5-dimethylpiperazin-1-yl)(2-fluoro-5-methoxyphenyl)methanone;
((3R,5R)-4-(2-fluoro-4-methoxybenzoyl)-3,5-dimethylpiperazin-1-yl)(5-hydroxy-1H-indol-3-yl)methanone;
((3R,5R)-3,5-dimethyl-4-(1-methyl-1H-indole-6-carbonyl)piperazin-1-yl)(2-fluoro-4-methoxyphenyl)methanone;
((2R,6R)-4-(2,4-dimethoxybenzoyl)-2,6-dimethylpiperazin-1-yl)(2-fluoro-4-methoxyphenyl)methanone;
((2R,6R)-4-(2-fluoro-4,5-dimethoxybenzoyl)-2,6-dimethylpiperazin-1-yl)(2-fluoro-4-methoxyphenyl)methanone;
((2R,6R)-4-(2-fluoro-4-(methylsulfonyl)benzoyl)-2,6-dimethylpiperazin-1-yl)(2-fluoro-4-methoxyphenyl)methanone;
N-(4-fluoro-3-((3R,5R)-4-(2-fluoro-4-methoxybenzoyl)-3,5-dimethylpiperazine-1-carbonyl)phenyl)acetamide;
((2R,6R)-4-(2-fluoro-4-methoxybenzoyl)-2,6-dimethylpiperazin-1-yl)(5-hydroxy-1H-indol-3-yl)methanone;
((2R,6R)-2,6-dimethylpiperazine-1,4-diyl)bis((4-methoxyphenyl)methanone);
(E)-1-((2R,6R)-4-(2-fluoro-4-methoxybenzoyl)-2,6-dimethylpiperazin-1-yl)-3-(4-hydroxyphenyl)prop-2-en-1-one;
(2-aminothiazol-5-yl)((3R,5R)-4-(2-fluoro-4-methoxybenzoyl)-3,5-dimethylpiperazin-1-yl)methanone;
((3S,5R)-4-(2-fluoro-4-methoxybenzoyl)-3,5-dimethylpiperazin-1-yl)(2-fluoro-5-methoxyphenyl)methanone;
or a pharmaceutically acceptable salt thereof.

12. A pharmaceutical composition comprising an effective amount of a compound or pharmaceutically acceptable salt of claim 1 and at least one pharmaceutically acceptable excipient.

13. A method of treating cancer in a subject in need of treatment,
comprising administering to said subject an effective amount of either the pharmaceutical composition of claim 12 or a compound or pharmaceutically acceptable salt thereof of a compound given by Formula I 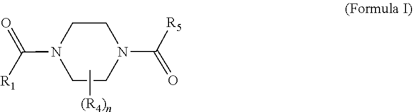
or a pharmaceutically acceptable salt thereof, wherein
R₁ is selected from the group consisting of 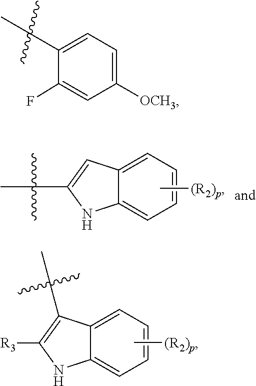
wherein
p is 0-2;
R₂ are individually the same or different and are selected from the group consisting of—OCH₃, —Cl, —OH, —CH₃, and —F;
R₃ are individually the same or different and are selected from the group consisting of—H or —CH₃;
n is 0-2;
R₄ are individually the same or different and are selected from —H or —CH₃;
R₅ is selected from the group consisting of: 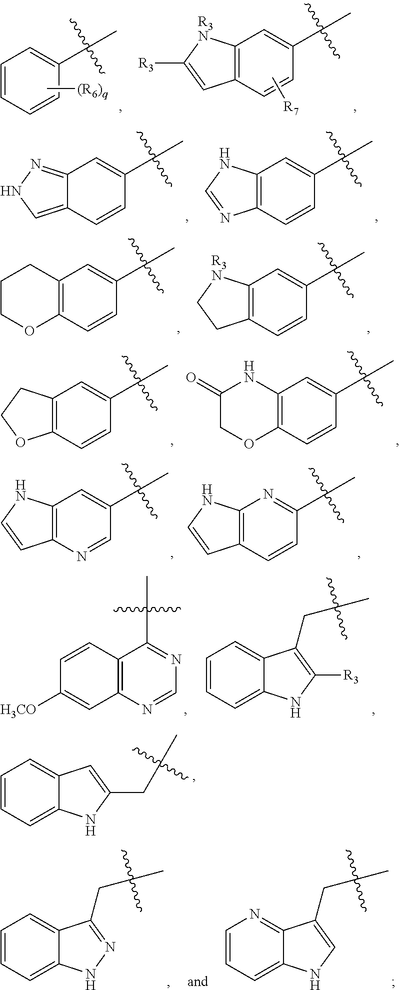
wherein
q is 0-3;
R₆ are independently the same or different and are selected from the group consisting of—F, —OCH₃, —OCHF₂, —SCH₃, —CH₃, —OCF₃, —OCH₂Ph, —OCH(CH₃)₂, —NHC(O)CH₃, —Cl, —OH, —N(CH₃)₂, —OEt, —CH₂CH₂OH, —S(O)₂CH₃, 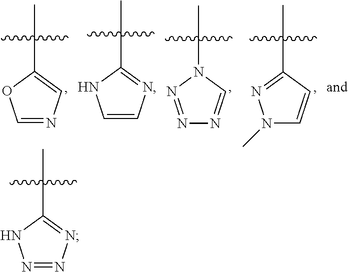
R₇ is —H, —F or —Cl.

14. A method of treating cancer in a subject in need of treatment,
comprising administering to said subject an effective amount of a compound or pharmaceutically acceptable salt of a compound given by Formula I 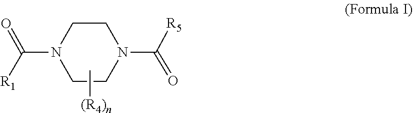
or a pharmaceutically acceptable salt thereof, wherein
R₁ is selected from the group consisting of 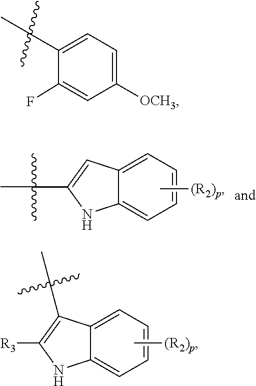
wherein
p is 0-2;
R₂ are individually the same or different and are selected from the group consisting of—OCH₃, —Cl, —OH, —CH₃, and —F;
R₃ are individually the same or different and are selected from the group consisting of—H or —
n is 0-2;
R₄ are individually the same or different and are selected from —H or —CH₃;
R₅ is selected from the group consisting of: 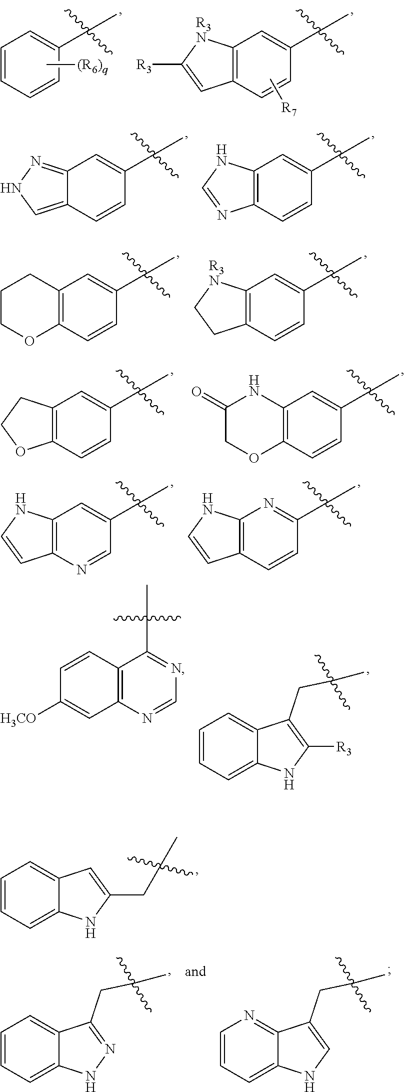
wherein
q is 0-3;
R₆ are independently the same or different and are selected from the group consisting of—F, —OCH₃, —OCHF₂, —SCH₃, —CH₃, —OCF₃, —OCH₂Ph —OCH(CH₃)₂, —NHC(O)CH₃, —Cl, —OH, —N(CH₃)₂, —OEt, —CH₂CH₂OH, —S(O)₂CH₃, 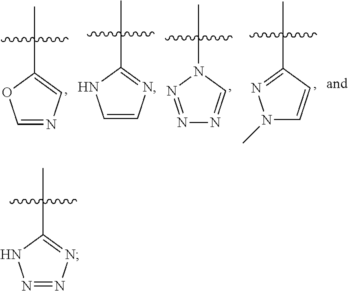
and
R₇ is —H, —F or —Cl or an effective amount of the pharmaceutical composition of claim 12.

15. A method of treating cancer in a subject in need thereof,
comprising:
detecting an altered CPS1 status in a biological sample from said subject containing cells of said cancer, and if said cancer has said altered CPS1 status, administering to said subject an effective amount of a compound or pharmaceutically acceptable salt of a compound given by Formula I 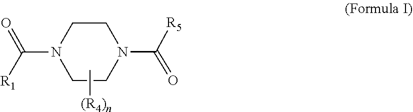
or a pharmaceutically acceptable salt thereof, wherein
R₁ is selected from the group consisting of 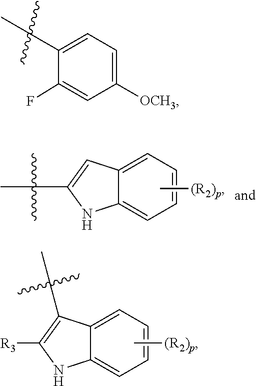
wherein
q is 0-2:
R₂ are individually the same or different and are selected from the group consisting of—OCH₃, —Cl, —OH, —CH₃, and —F;
R₃ are individually the same or different and are selected from the group consisting of—H or —CH₃;
n is 0-2;
R₄ are individually the same or different and are selected from —H or —CH₃;
R₅ is selected from the group consisting of: 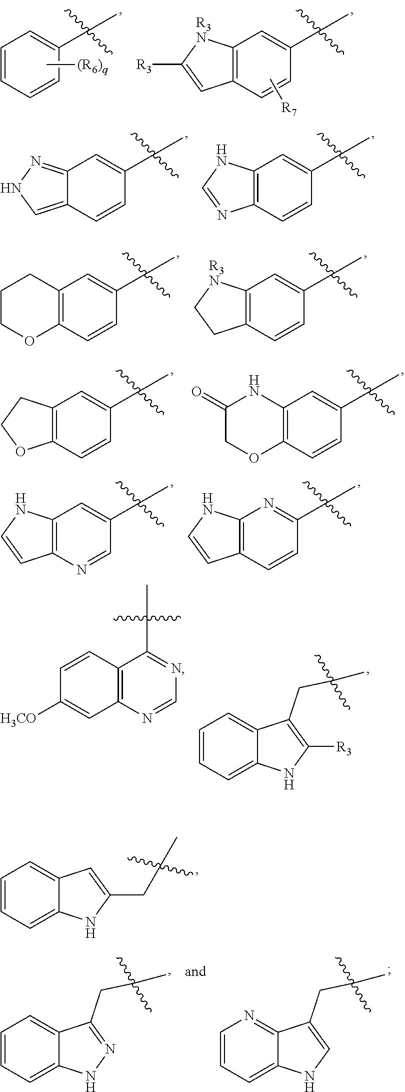
wherein
q is 0-3;
R₆ are independently the same or different and are selected from the group consisting of—F, —OCH₃, —OCHF₂, —SCH₃, —CH₃, —OCF₃, —OCH₂Ph —OCH(CH₃)₂, —NHC(O)CH₃, —Cl, —OH, —N(CH₃)₂, —OEt, —CH₂CH₂OH, —S(O)₂CH₃, 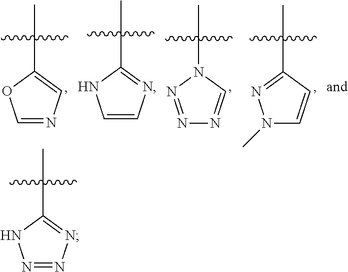
R₇ is —H, —F or —Cl or an effective amount of the pharmaceutical composition of claim 12.

16. The method of claim 15, wherein said altered CPS1 status is increased expression of CPS1.

17. Use of a compound or pharmaceutically acceptable salt of claim 1 in the manufacture of a medicament for treatment of cancer.

18. Use of a compound or pharmaceutically acceptable salt of a compound given by Formula I 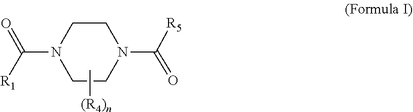
or a pharmaceutically acceptable salt thereof, wherein
R₁ is selected from the group consisting of 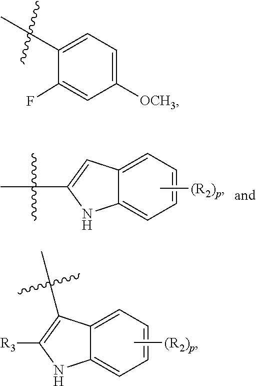
wherein p is 0-2;
R₂ are individually the same or different and are selected from the group consisting of—OCH₃, —Cl, —OH, —CH₃, and —F;
R₃ are individually the same or different and are selected from the group consisting of—H or —CH
n is 0-2:
R₄ are individually the same or different and are selected from —H or —CH₃;
R₅ is selected from the group consisting of: 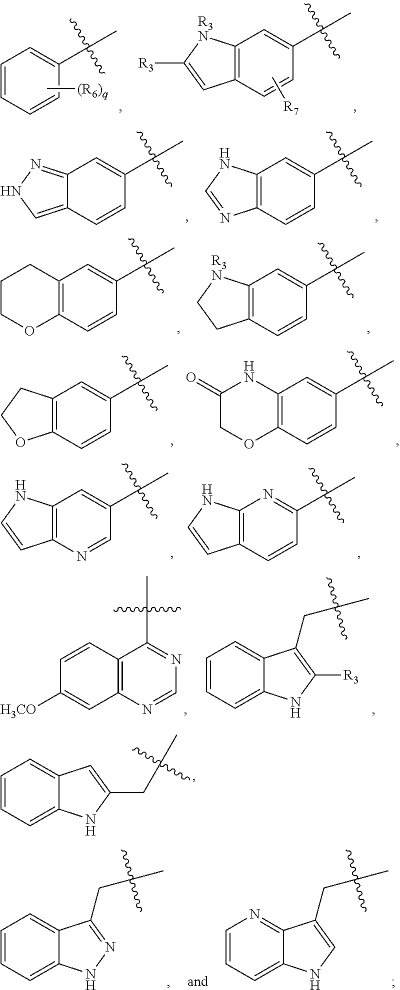
wherein
q is 0-3;
R₆ are independently the same or different and are selected from the group consisting of—F, —OCH₃, —OCHF₂, —SCH₃, —CH₃, —OCF₃, —OCH₂Ph, —OCH(CH₃)₂, —NHC(O)CH₃, —Cl, —OH, —N(CH₃)₂, —OEt, —CH₂CH₂OH, —S(O)₂CH₃, 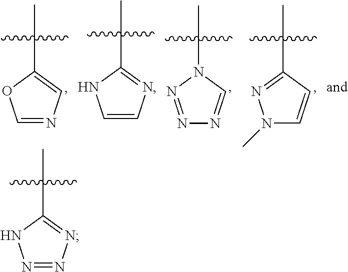
R₇ is —H, —F or —Cl or of the pharmaceutical composition of claim 12 for the treatment of cancer.
